(19) United States
(12) Patent Application Publication
(10) Pub. No.: US 2025/0146087 A1
(43) Pub. Date: May 8, 2025

(54) TUMOR MICROBIOME ANALYSIS AS A PROGNOSTIC INDICATOR OF PROSTATE CANCER

(71) Applicant: UEA ENTERPRISES LIMITED, Norwich Norfolk (GB)

(72) Inventors: Colin Stephen Cooper, Norwich Norfolk (GB); Daniel Simon Brewer, Norwich Norfolk (GB); Rachel Hurst, Norwich Norfolk (GB); Abraham Farag Salah Gihawi, Norwich Norfolk (GB); Justin Joseph O'Grady, Norwich Norfolk (GB); John Richard Wain, Norwich Norfolk (GB); Emma Jane Meader, Norwich Norfolk (GB); Ghanasyam Rallapalli, Norwich Norfolk (GB)

(21) Appl. No.: 18/730,566

(22) PCT Filed: Jan. 18, 2023
(86) PCT No.: PCT/EP2023/051155

§ 371 Date: Jul. 19, 2024

(30) Foreign Application Priority Data
Jan. 19, 2022 (GB) 2200682.9

(51) Int. Cl. C12Q 1/689; C12N 1/20; C12Q 1/686; C12Q 1/6886; C12R 1/01

(52) U.S. Cl. CPC C12Q 1/689; C12N 1/205; C12Q 1/686; C12Q 1/6886; C12Q 2600/118; C12R 2001/01

(57) ABSTRACT

The present invention relates to prostate cancer (PC), in particular the identification of certain bacteria in prostate cancer tissues or liquid biopsies and the use of such identification as a prognostic indicator of cancer progression. The present invention also relates to cultures of newly identified bacteria and means for identifying such bacteria in biological samples.

### FIELD OF THE INVENTION

The present invention relates to prostate cancer (PC), in particular the identification of certain bacteria in prostate cancer tissues or liquid biopsies and the use of such identification as a prognostic indicator of cancer progression. The present invention also relates to cultures of newly identified bacteria and means for identifying such bacteria in biological samples.

### INTRODUCTION

Prostate cancer exhibits extreme clinical heterogeneity; 10-year survival rates following diagnosis approach 84%, yet prostate cancer is still responsible for 13% of all cancer deaths in men in the UK [1]. Coupled with the high rates of diagnosis, prostate cancer is more often a disease that men die with rather than from. This illustrates the need for clinically implementable tools able to selectively identify those men that can be safely removed from treatment pathways without missing those men harbouring disease that requires intervention.

An opportune point to intervene or supplement current clinical practices would be prior to an initial biopsy in men suspected of having prostate cancer, reducing costs to men, healthcare systems and providers alike. In current clinical practice men are selected for further investigations for prostate cancer if they have an elevated PSA (≥4 ng/mL) and an adverse finding on digital rectal examination (DRE) or lower urinary tract symptoms; other factors such as age and ethnicity are also considered [2,3,4].

D'Amico stratification [5], which classifies patients as Low-Intermediate- or High-risk of PSA-failure post-radical therapy, is based on Gleason Score (Gs) [6], PSA and clinical stage, and has been used as a framework for guidelines issued in the UK, Europe and USA [7,8,9]. Low-risk, and some favourable Intermediate-risk patients are generally offered Active Surveillance (AS) while unfavourable Intermediate-, and High-risk patients are considered for radical therapy [7,10]. Other classification systems such as CAPRA score [11] use additional clinical information, assigning simple numeric values based on age, pre-treatment PSA, Gleason Score, percentage of biopsy cores positive for cancer and clinical stage for an overall 0-10 CAPRA score. The CAPRA score has shown favourable prediction of PSA-free survival, development of metastasis and prostate cancer-specific survival [12].

However, the rates of negative biopsies in men with a clinical suspicion of prostate cancer are overwhelming; a recent population-level study of 419,582 men from Martin et al observed that 60% of all biopsies in the control arm of the Cluster Randomized Trial of PSA Testing for Prostate Cancer (CAP) were negative for prostate cancer [13], similar to the rates observed by Donovan et al as part of the ProtecT trial [14]. Needle biopsy is invasive, and not without complications: 44% of patients report pain as a result of the biopsy, and detection of clinically insignificant disease can result in years of monitoring, causing patients undue stress [4]. Multiparametric MRI (MP-MRI) has been developed as a triage tool to reduce the rates of negative biopsy and its use has become increasingly widespread since its validation [15]. However, MP-MRI is relatively expensive and has shown a high rate of inter-operator and inter-machine variability, leading to mpMRI missing up to 28% of clinically significant diseases in practice [4,16,17,18].

The interconnected nature of the male urological system makes it an ideal candidate for liquid biopsy and non-invasive biomarkers for prostate cancer. There is sizeable interest in the development of such non-invasive tests and classifiers capable of reducing the rates of initial biopsy in men, whilst retaining the sensitivity to detect aggressive disease. Single-gene or expression panels of few genes, such as the PCA3 [19], SelectMDx [20], ExoDx Prostate(IntelliScore) [21] tests have published promising results to date for the non-invasive detection of significant disease (Gleason score (Gs)≥7).

Prostate cancer is the most common non-skin malignancy in men in developed countries, with over 250,000 deaths per year worldwide. It is more common in black men [22] and is becoming an increasingly important issue in North and East Asia [23]. Although the underlying causes of most prostate cancer remain obscure, genetic inheritance has an established role [24] and chronic inflammation has been proposed as an aetiological factor [25,26,27]. Bacteria can be found in the urogenital tract and in prostate tissue [25], and alterations in the bacteria present in urine and faeces have been associated with the development of prostate cancer [26,28]. The clinical course of prostate cancer progression is highly heterogeneous and critical decisions are made about the likelihood of disease progression based on information obtained at presentation, dictating treatment choice. Current guidelines in the UK, USA, and Europe incorporate an assessment of low, intermediate or high D'Amico progression risk, which is calculated using levels of the serum biomarker PSA (Prostate Specific Antigen), the histopathological Gleason score determined following prostate needle biopsy, and clinical stage [29]. Problems associated with obtaining representative cancer samples using needle biopsy have generated considerable interest in developing complementary non-invasive tests in order to detect clinically significant prostate cancer that will progress. Examining prostate-derived material harvested from urine has the potential to achieve this. Material secreted by the prostate gland frequently appears in the urine, and reflux of urine into the prostate is well established suggesting the existence of a prostate-urine loop [30,31,32]. Several urine biomarkers identified include assessment of gene methylation [33], or gene expression profiles including the PUR test [34] and combinations of such markers [35]. However, currently none of these tests are in widespread clinical use. The challenge remains to find a combination of biomarkers and clinical data that at initial patient assessment prior to biopsy can reliably predict the presence of prostate cancer that will progress clinically and become potentially life threatening: also referred to as aggressive prostate cancer.

There is a link between the presence of prostate cancer and distinct microbial profiles in the gastrointestinal tract [28,36,37] and encouragingly different microbes are present in prostate tissue from patients with different Gleason grades [38]. The bacteria identified are known to induce inflammation [39] or transformation [25] in model systems (reviewed in refs 25, 39 and 40). A variety of potential neoplasm promoting molecular mechanisms have been described for bacteria including the production of DNA-binding genotoxic agent [39] and chronic exposure to ROS or inhibition of tumour suppressor p53 [40]. Subversion of metabolic processes within human cells has been reported [25, 28, 36], including production of metabolites such as short-chain fatty acids (e.g., propionic acid produced by Cutibacterium acnes) which could impact on locally infiltrated T-cell populations and immunosuppression [25, 36]. Despite this, establishing a firm link between the presence of micro-organisms and the development of prostate cancer has remained elusive, hindered by problems of sample contamination and the limited characterisation of the microbiota present.

In the current study we have used independent sample collections of different tissue types; controls selected from disease staged individuals; population level 16S and mRNA sequencing as well as culture and whole genome sequence data. This allowed us to use non-biased “tree of life” [41]methodology to classify the novel bacteria isolated and seek associations with disease progression.

### SUMMARY OF THE INVENTION

The present invention provides a method of detecting the presence of one or more bacteria selected from the genera Fenollaria, Ezakiella, Peptoniphilus, Porphyromonas, Anaerococcus, Varibaculum and Fusobacterium in a biological sample from the patient. The present invention also provides a method of detecting the presence of one or more bacteria selected from the genera Fenollaria, Peptoniphilus, Porphyromonas, Anaerococcus and Fusobacterium in a biological sample from the patient.

In some embodiments, the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum. In some embodiments, the one or more bacteria are selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

In some embodiments, the one or more bacteria are selected from: Fenollaria sporofastidiosus, Peptoniphilus rachelemmaiella, Anaerococcus prevotii, Porphyromonas bobii, Porphyromonas asaccharolytica and Fusobacterium nucleatum. In some embodiments, the one or more bacteria are selected from: Fenollaria sporofastidiosus, Peptoniphilus harei, Anaerococcus prevotii, Porphyromonas bobii, Porphyromonas asaccharolytica and Fusobacterium nucleatum. In some embodiments, the one or more bacteria are selected from: Fenollaria sporofastidiosus, Peptoniphilus rachelemmaiella, Peptoniphilus harei, Anaerococcus prevotii, Porphyromonas asaccharolytica and Fusobacterium nucleatum. In some embodiments, the one or more bacteria are selected from: Fenollaria sporofastidiosus, Peptoniphilus rachelemmaiella, Peptoniphilus harei, Anaerococcus prevotii, Porphyromonas bobii and Fusobacterium nucleatum. In some embodiments, the one or more bacteria are selected from: Fenollaria sporofastidiosus, Peptoniphilus rachelemmaiella, Anaerococcus prevotii, Porphyromonas bobii and Fusobacterium nucleatum. In some embodiments, the one or more bacteria are selected from: Fenollaria sporofastidiosus, Peptoniphilus rachelemmaiella, Anaerococcus prevotii, Porphyromonas asaccharolytica and Fusobacterium nucleatum. In some embodiments, the one or more bacteria are selected from: Fenollaria sporofastidiosus, Peptoniphilus harei, Anaerococcus prevotii, Porphyromonas bobii and Fusobacterium nucleatum. In some embodiments, the one or more bacteria are selected from: Fenollaria sporofastidiosus, Peptoniphilus harei, Anaerococcus prevotii, Porphyromonas asaccharolytica and Fusobacterium nucleatum.

In some embodiments, the method comprises detecting the presence of at least 1, 2, 3, 4, 5, 6, 7, 8, 9 or 10 different bacteria. In some embodiments, the method comprises detecting all of the species recited in a given list.

In some embodiments, the method comprises detecting each of Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum. In some embodiments, the method comprises detecting each of Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

In some embodiments, the method further comprises detecting the presence of one or more bacteria selected from Peptoniphilus coxii, Anaerococcus lactolyticus and Porphyromonas bennonis. In some embodiments, the method further comprises detecting the presence of Varibaculum prostatecancerukia.

In some embodiments, one or more of the bacteria are selected from: Fenollaria sporofastidiosus (identified by ECACC accession number 21092201 or DSMZ accession number DSM 34056), Peptoniphilus rachelemmaiella (identified by ECACC accession number 21092202 or DSMZ accession number DSM 34055) and Porphyromonas bobii (identified by ECACC accession number 21092204 or DSMZ accession number DSM 34063). In some embodiments, the bacteria is Varibaculum prostatecancerukia (identified by ECACC accession number 21092203 or DSMZ accession number DSM 34057).

In some embodiments, the biological sample is processed prior to determining the presence of the one or more bacteria in the biological sample. In some embodiments, determining the presence of the one or more bacteria comprises extracting a nucleic acid from the biological sample. In some embodiments, determining the presence of the one or more bacteria comprises extracting bacterial DNA from the biological sample. In some embodiments, determining the presence of the one or more bacteria comprises extracting bacterial RNAfrom the biological sample. In some embodiments, determining the presence of the one or more bacteria comprises extracting one or more bacterial proteins from the biological sample. In some embodiments, determining the presence of the one or more bacteria comprises extracting one or more bacteria-specific carbohydrates from the biological sample.

In some embodiments, the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample). In some embodiments, the biological sample is a urine sample.

In some embodiments, the presence of the one or more bacteria is determined using techniques such as quantitative polymerase chain reaction (qPCR) or probe-based detection assays including, but not limited to, NanoString®. In some embodiments, the presence of the one or more bacteria is determined using quantitative polymerase chain reaction (qPCR) or probe-based detection assays (such as NanoString®) wherein the qPCR or probe-based detection assays are conducted on one or more of bacterial nucleic acids in the biological sample.

In some embodiments, determining the presence of the one or more bacteria comprises detecting one or more bacterial proteins in a sample. In some embodiments, determining the presence of the one or more bacteria comprises detecting one or more bacterial-specific carbohydrates. In some embodiments, the presence of the one or more bacteria is determined using matrix-assisted laser desorption/ionization-time of flight mass spectrometry (MALDI-TOF).

In some embodiments, determining the presence of the one or more bacteria comprises detecting one or more bacterial metabolites in a sample. In some embodiments, determining the presence of the one or more bacteria comprises detecting one or more anaerobic bacterial metabolites in a sample. In some embodiments, determining the presence of the one or more bacteria comprises detecting one or more bacterial metabolites using a kit comprising means for detecting one or more bacterial metabolites. In some embodiments, the kit comprising means for detecting one or more bacterial metabolites can comprise a dipstick comprising one or more binding molecules (e.g. antibodies) specific for one or more bacterial metabolites. In some embodiments, the one or more bacterial metabolites are detectable in a urine sample.

In some embodiments, the presence of the one or more bacteria is determined using quantitative polymerase chain reaction (qPCR) or a probe-based detection assay (such as NanoString®) using one or more primers comprising a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. In some embodiments, the presence of the one or more bacteria is determined using quantitative polymerase chain reaction (qPCR) or a probe-based detection assay (such as NanoString®) using one or more primers consisting of a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

The present invention also provides a method of providing a cancer prognosis in a patient based on the presence of one or more bacteria in a biological sample from the patient, comprising:
(a) detecting the presence of one or more bacteria selected from the genera Fenollaria, Ezakiella, Peptoniphilus, Porphyromonas, Anaerococcus, Varibaculum and Fusobacterium in a biological sample from the patient, and
(b) providing a prognosis for the patient, wherein the patient has a poor prognosis if one or more of the bacteria are present in the biological sample.

The present invention also provides a method of providing a cancer prognosis in a patient based on the presence of one or more bacteria in a biological sample from the patient, comprising:
(a) detecting the presence of one or more bacteria selected from the genera Fenollaria, Peptoniphilus, Porphyromonas, Anaerococcus and Fusobacterium in a biological sample from the patient, and
(b) providing a prognosis for the patient, wherein the patient has a poor prognosis if one or more of the bacteria are present in the biological sample.

In some embodiments, the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum. In some embodiments, the one or more bacteria are selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

In some embodiments, the one or more bacteria are selected from: Fenollaria sporofastidiosus, Peptoniphilus rachelemmaiella, Anaerococcus prevotii, Porphyromonas bobii, Porphyromonas asaccharolytica and Fusobacterium nucleatum. In some embodiments, the one or more bacteria are selected from: Fenollaria sporofastidiosus, Peptoniphilus harei, Anaerococcus prevotii, Porphyromonas bobii, Porphyromonas asaccharolytica and Fusobacterium nucleatum. In some embodiments, the one or more bacteria are selected from: Fenollaria sporofastidiosus, Peptoniphilus rachelemmaiella, Peptoniphilus harei, Anaerococcus prevotii, Porphyromonas asaccharolytica and Fusobacterium nucleatum. In some embodiments, the one or more bacteria are selected from: Fenollaria sporofastidiosus, Peptoniphilus rachelemmaiella, Peptoniphilus harei, Anaerococcus prevotii, Porphyromonas bobii and Fusobacterium nucleatum. In some embodiments, the one or more bacteria are selected from: Fenollaria sporofastidiosus, Peptoniphilus rachelemmaiella, Anaerococcus prevotii, Porphyromonas bobii and Fusobacterium nucleatum. In some embodiments, the one or more bacteria are selected from: Fenollaria sporofastidiosus, Peptoniphilus rachelemmaiella, Anaerococcus prevotii, Porphyromonas asaccharolytica and Fusobacterium nucleatum. In some embodiments, the one or more bacteria are selected from: Fenollaria sporofastidiosus, Peptoniphilus harei, Anaerococcus prevotii, Porphyromonas bobii and Fusobacterium nucleatum. In some embodiments, the one or more bacteria are selected from: Fenollaria sporofastidiosus, Peptoniphilus harei, Anaerococcus prevotii, Porphyromonas asaccharolytica and Fusobacterium nucleatum.

In some embodiments, the method comprises detecting the presence of at least 1, 2, 3, 4, 5, 6, 7, 8, 9 or 10 different bacteria. In some embodiments, the method comprises detecting all of the species recited in a given list.

In some embodiments, the method comprises detecting each of Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum. In some embodiments, the method comprises detecting each of Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

In some embodiments, the method further comprises detecting the presence of one or more bacteria selected from Peptoniphilus coxii, Anaerococcus lactolyticus and Porphyromonas bennonis. In some embodiments, the method further comprises detecting the presence of Varibaculum prostatecancerukia.

In some embodiments, one or more of the bacteria are selected from: Fenollaria sporofastidiosus (identified by ECACC accession number 21092201 or DSMZ accession number DSM 34056), Peptoniphilus rachelemmaiella (identified by ECACC accession number 21092202 or DSMZ accession number DSM 34055) and Porphyromonas bobii (identified by ECACC accession number 21092204 or DSMZ accession number DSM 34063). In some embodiments, the bacteria is Varibaculum prostatecancerukia (identified by ECACC accession number 21092203 or DSMZ accession number DSM 34057).

In some embodiments, a poor prognosis is associated with an increased risk of elevated rates of metastases. In some embodiments, a poor prognosis is associated with a progression of the cancer stage. In some embodiments, a poor prognosis is associated with an increase in prostate-specific antigen (PSA) and/or PSA failure. In some embodiments, a poor prognosis is associated with an increased cancer risk score, optionally wherein the risk score is a Gleason risk score.

In some embodiments, the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample). In some embodiments, the biological sample is a urine sample.

In some embodiments, the method is used to determine whether a patient should be biopsied. In some embodiments, the biological sample is processed prior to determining the presence of the one or more bacteria in the biological sample. In some embodiments, determining the presence of the one or more bacteria comprises extracting a nucleic acid from the biological sample. In some embodiments, determining the presence of the one or more bacteria comprises extracting bacterial DNA from the biological sample. In some embodiments, determining the presence of the one or more bacteria comprises extracting bacterial RNA from the biological sample. In some embodiments, determining the presence of the one or more bacteria comprises detecting one or more bacterial proteins in a sample. In some embodiments, determining the presence of the one or more bacteria comprises detecting one or more bacterial-specific carbohydrates. In some embodiments, the presence of the one or more bacteria is determined using quantitative polymerase chain reaction (qPCR) or a probe-based detection assay (such as NanoString®).

In some embodiments, the presence of the one or more bacteria is determined using quantitative polymerase chain reaction (qPCR) or a probe-based detection assay (such as NanoString®) using one or more primers comprising a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. In some embodiments, the presence of the one or more bacteria is determined using quantitative polymerase chain reaction (qPCR) or a probe-based detection assay (such as NanoString®) using one or more primers consisting of a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ 40 ID NO:15, SEQ ID NO:16, SEQ ID NO:17, SEQ ID NO:18, SEQ ID NO:19 and SEQ ID NO:20.

In some embodiments, the sample is from a human.

The present invention also provides a method of treating a cancer in a patient in need thereof, comprising providing a prognosis of a patient's cancer using a method according to the invention, and administering to the patient a therapy for treating cancer. The present invention also provides a method of treating cancer in a patient in need thereof, wherein the prognosis of the patient has been determined using a method according to the invention, comprising administering to the patient a therapy for treating cancer.

In some embodiments, the cancer is selected from the list: Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Central Nervous System (Brain Cancer), Basal Cell Carcinoma, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors (Lung Cancer), Burkitt Lymphoma—see Non-Hodgkin Lymphoma, Carcinoid Tumor (Gastrointestinal), Cardiac (Heart) Tumors, Medulloblastoma and Other CNS Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Embryonal Tumors, Medulloblastoma, Endometrial Cancer, Ependymoma, Esophageal Cancer, Esthesioneuroblastoma, Ewing Sarcoma (Bone Cancer), Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Heart Tumors, Hepatocellular (Liver) Cancer, Histiocytosis, Langerhans Cell Hodgkin Lymphoma, Hypopharyngeal Cancer, Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Melanoma, Intraocular (Eye), Merkel Cell Carcinoma, Mesothelioma, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, Acute (AML), Myeloproliferative Neoplasms, Chronic Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma, Childhood Rhabdomyosarcoma (Soft Tissue Sarcoma), Childhood Vascular Tumors (Soft Tissue Sarcoma), Ewing Sarcoma (Bone Cancer), Osteosarcoma (Bone Cancer), Soft Tissue Sarcoma, Uterine Sarcoma, Sezary Syndrome, Skin Cancer, Small Cell Lung Cancer, Small Intestine Cancer, Squamous Cell Carcinoma, Squamous Neck Cancer with Occult Primary, Metastatic, Stomach (Gastric) Cancer, T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Ureter and Renal Pelvis, Transitional Cell Cancer (Kidney (Renal Cell) Cancer, Urethral Cancer, Uterine Cancer, Vaginal Cancer, Vascular Tumors, Vulvar Cancer, Wilms Tumor, Myxofibrosarcoma, Leiomyosarcoma, Pleomorphic Sarcoma and Hepatopancreatobiliary cancer.

In some embodiments, the cancer is prostate cancer. In some embodiments, the therapy for cancer comprises surgery, brachytherapy, active surveillance, chemotherapy, hormone therapy, immunotherapy and/or radiotherapy. In some embodiments, the therapy for cancer comprises chemotherapy, hormone therapy and/or immunotherapy. In some embodiments, the chemotherapy comprises administration of one or more agents selected from the list: abiraterone acetate, apalutamide, bicalutamide, cabazitaxel, bicalutamide, degarelix, docetaxel, leuprolide acetate, enzalutamide, apalutamide, flutamide, goserelin acetate, mitoxantrone, nilutamide, sipuleucel T, radium 223 dichloride and docetaxel. In some embodiments, the therapy for cancer comprises resection of all or part of a tumour. In some embodiments, the cancer is prostate cancer and the therapy for cancer comprises resection of all or part of the prostate gland and/or all or part of a prostate tumour.

The present invention also provides a nucleotide primer comprising a sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

The present invention also provides a nucleotide primer consisting of a sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

The present invention also provides a kit for determining the presence of one or more bacteria in a biological sample, comprising means for detecting one or more bacteria selected from the genera Fenollaria, Ezakiella, Peptoniphilus, Porphyromonas, Anaerococcus, Varibaculum and Fusobacterium in a biological sample.

The present invention also provides a kit for determining the presence of one or more bacteria in a biological sample, comprising means for detecting one or more bacteria selected from the genera Fenollaria, Peptoniphilus, Porphyromonas, Anaerococcus and Fusobacterium in a biological sample.

In some embodiments of kits of the invention, the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

In some embodiments of kits of the invention, the one or more bacteria are selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum. In some embodiments of kits of the invention, the one or more bacteria are selected from: Fenollaria sporofastidiosus, Peptoniphilus rachelemmaiella, Anaerococcus prevotii, Porphyromonas bobii, Porphyromonas asaccharolytica and Fusobacterium nucleatum. In some embodiments of kits of the invention, the one or more bacteria are selected from: Fenollaria sporofastidiosus, Peptoniphilus harei, Anaerococcus prevotii, Porphyromonas bobii, Porphyromonas asaccharolytica and Fusobacterium nucleatum. In some embodiments of kits of the invention, the one or more bacteria are selected from: Fenollaria sporofastidiosus, Peptoniphilus rachelemmaiella, Peptoniphilus harei, Anaerococcus prevotii, Porphyromonas asaccharolytica and Fusobacterium nucleatum. In some embodiments of kits of the invention, the one or more bacteria are selected from: Fenollaria sporofastidiosus, Peptoniphilus rachelemmaiella, Peptoniphilus harei, Anaerococcus prevotii, Porphyromonas bobii and Fusobacterium nucleatum. In some embodiments of kits of the invention, the one or more bacteria are selected from: Fenollaria sporofastidiosus, Peptoniphilus rachelemmaiella, Anaerococcus prevotii, Porphyromonas bobii and Fusobacterium nucleatum. In some embodiments of kits of the invention, the one or more bacteria are selected from: Fenollaria sporofastidiosus, Peptoniphilus rachelemmaiella, Anaerococcus prevotii, Porphyromonas asaccharolytica and Fusobacterium nucleatum. In some embodiments of kits of the invention, the one or more bacteria are selected from: Fenollaria sporofastidiosus, Peptoniphilus harei, Anaerococcus prevotii, Porphyromonas bobii and Fusobacterium nucleatum. In some embodiments of kits of the invention, the one or more bacteria are selected from: Fenollaria sporofastidiosus, Peptoniphilus harei, Anaerococcus prevotii, Porphyromonas asaccharolytica and Fusobacterium nucleatum.

In some embodiments of kits of the invention, the kit comprises means for detecting the presence of at least 1, 2, 3, 4, 5, 6, 7, 8, 9 or 10 different bacteria. In some embodiments of kits of the invention, the kit comprises means for detecting all of the species recited in a given list.

In some embodiments of kits of the invention, the kit comprises means for detecting each of Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum. In some embodiments of kits of the invention, the kit comprises means for detecting each of Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum. In some embodiments of kits of the invention, the kit further comprises means for detecting the presence of one or more bacteria selected from Peptoniphilus coxii, Anaerococcus lactolyticus and Porphyromonas bennonis. In some embodiments of kits of the invention, the kit further comprises means for detecting the presence of Varibaculum prostatecancerukia.

In some embodiments of kits of the invention, one or more of the bacteria are selected from: Fenollaria sporofastidiosus (identified by ECACC accession number 21092201 or DSMZ accession number DSM 34056), Peptoniphilus rachelemmaiella (identified by ECACC accession number 21092202 or DSMZ accession number DSM 34055) and Porphyromonas bobii (identified by ECACC accession number 21092204 or DSMZ accession number DSM 34063). In some embodiments of kits of the invention, the bacteria is Varibaculum prostatecancerukia (identified by ECACC accession number 21092203 or DSMZ accession number DSM 34057).

In some embodiments of kits of the invention, the means for detecting is a biosensor or specific binding molecule. In some embodiments of kits of the invention, the biosensor is an electrochemical, electronic, piezoelectric, gravimetric, pyroelectric biosensor, ion channel switch, evanescent wave, surface plasmon resonance or biological biosensor. In some embodiments of kits of the invention, the biosensor is an antigen-specific binding molecule capable of specifically binding to the one or more bacteria, optionally wherein the antigen-specific binding molecule is an antibody or fragment thereof. In some embodiments of kits of the invention, the biosensor is an antigen-specific binding molecule capable of specifically binding to the one or more bacterial metabolites, optionally wherein the antigen-specific binding molecule is an antibody or fragment thereof. In some embodiments of kits of the invention, the biosensor is an antigen-specific binding molecule capable of specifically binding to the one or more anaerobic bacterial metabolites, optionally wherein the antigen-specific binding molecule is an antibody or fragment thereof. In some embodiments, the one or more bacterial metabolites are detectable in a urine sample.

In some embodiments of kits of the invention, the means for detecting is quantitative polymerase chain reaction (qPCR) or a probe-based detection assay (such as NanoString®). In some embodiments of kits of the invention, the means for detecting is quantitative polymerase chain reaction (qPCR) or a probe-based detection assay (such as NanoString®) and the kit comprises one or more primers comprising a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. In some embodiments of kits of the invention, the means for detecting is quantitative polymerase chain reaction (qPCR) or a probe-based detection assay (such as NanoString®) and the kit comprises one or more primers consisting of a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

The present invention also provides a cell culture comprising bacteria Varibaculum prostatecancerukia identified by ECACC deposit number 21092203 or DSMZ accession number DSM 34057.

The present invention also provides a cell culture comprising bacteria Fenollaria sporofastidiosus identified by ECACC deposit number 21092201 or DSMZ accession number DSM 34056.

The present invention also provides a cell culture comprising bacteria Peptoniphilus rachelemmaiella identified by ECACC deposit number 21092202 or DSMZ accession number DSM 34055.

The present invention also provides a cell culture comprising bacteria Porphyromonas bobii identified by ECACC deposit number 21092204 or DSMZ accession number DSM 34063.

The present invention also provides use of a cell culture according to the invention in providing a prognosis of cancer in a patient.

The present invention also provides a computer apparatus configured to perform a method according to the invention or a computer readable medium programmed to perform a method according to the invention.

In some embodiments, one or more of the bacteria comprise 1, 2, 3, 4, 5, 6 or 7 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum. In some embodiments, one or more of the bacteria comprise 2 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum. In some embodiments, one or more of the bacteria comprise 3 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum. In some embodiments, one or more of the bacteria comprise 4 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum. In some embodiments, one or more of the bacteria comprise 5 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum. In some embodiments, one or more of the bacteria comprise 6 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum. In some embodiments, one or more of the bacteria comprise all 7 bacteria Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

The present invention also provides a nucleotide primer comprising a sequence between 5 and 25 nucleotides in length that is substantially complementary or reverse complementary to a corresponding length portion of SEQ ID NO:25. In some embodiments, the primer is capable of detecting the presence of Varibaculum prostatecancerukia in a biological sample.

The present invention also provides a nucleotide primer comprising a sequence between 5 and 25 nucleotides in length that is substantially complementary or reverse complementary to a corresponding length portion of SEQ ID NO:26. In some embodiments, the primer is capable of detecting the presence of Fenollaria sporofastidiosus in a biological sample.

The present invention also provides a nucleotide primer comprising a sequence between 5 and 25 nucleotides in length that is substantially complementary or reverse complementary to a corresponding length portion of a sequence selected from the list consisting of SEQ ID NOs:27-67. In some embodiments, the primer is capable of detecting the presence of Peptoniphilus rachelemmaiella in a biological sample.

The present invention also provides a nucleotide primer comprising a sequence between 5 and 25 nucleotides in length that is substantially complementary or reverse complementary to a corresponding length portion of a sequence selected from the list consisting of SEQ ID NOs:68-84. In some embodiments, the primer is capable of detecting the presence of Porphyromonas bobii in a biological sample.

In some embodiments, the presence of the one or more bacteria is determined using quantitative polymerase chain reaction (qPCR) or a probe-based detection assay (such as NanoString®) using one or more primers according to the invention. In some embodiments of kits of the invention, the means for detecting is quantitative polymerase chain reaction (qPCR) or a probe-based detection assay (such as NanoString®) using one or more primers according to the invention.

The present invention also provides use of a cell culture according to the invention in providing a prognosis of cancer in a patient. The present invention also provides a computer apparatus configured to perform a method according to the invention or, a computer readable medium programmed to perform a method according to the invention.

In some embodiments of the invention methods of the invention may comprise measuring one or more genera of bacteria in combination with one or more species of bacteria. In some embodiments of the invention kits of the invention may comprise means for measuring one or more genera of bacteria (for example pan-genera primers or probes) in combination with means for measuring one or more species of bacteria (for example species-specific primers or probes). Throughout this specification where a list of particular species of bacteria are specified as a narrower embodiment of methods or kits reciting a list of genera of bacteria, the methods or kits may comprise one or more genera from the specified list of genera in combination with one or more species from the specified list of species. The lists of particular species are intended to define species which may be used in combination with the other species and/or genera in a given list or as an alternative to use of the entire genera. For example, a method reciting use of genera Fenollaria, Ezakiella, Peptoniphilus, Porphyromonas, Anaerococcus, Varibaculum and Fusobacterium may include the particular species Fenollaria sporofastidiosus in place of the genera Fenollaria or may include Fenollaria sporofastidiosus in combination with any one or more other genera listed. Similarly, the method may include any one or more of Peptoniphilus rachelemmaiella, Peptoniphilus harei and Peptoniphilus coxii in place of the genera Peptoniphilus or any one or more of Peptoniphilus rachelemmaiella, Peptoniphilus harei and Peptoniphilus coxii in combination with any one or more of the other genera listed.

### BRIEF DESCRIPTION OF THE FIGURES

FIG. 1—Presence of bacteria in urine cell sediment and association with prostate cancer clinical grade. A) Example fluorescence microscopy images of urine cell sediment sample stained with DAPI are negative (left) and positive (right) for background fluorescence bacteria staining, scale bar 30 μm. In the left-hand panel only DAPI fluoresce corresponding to eukaryotic nuclei can be observed. The presence of background DAPI fluorescence correlated to the presence of bacterial 16S PCR products (χ²-test, df=1, P=9.3×10⁻¹⁰) (Table 5). B) Example scanning electron microscopy images of the urine from two patients exhibiting staining for background DAPI fluorescence showing the morphological appearance of bacterial cells. Scale bar 1 μm. Scanning electron microscopy performed for 5 additional patients gave similar appearance. C) Significant correlation of D'Amico clinical category and the presence of background DAPI stained bacteria fluorescence in post-DRE urine. Data are presented as percentage positive for bacteria/microorganisms. Discovery set (n=215 samples, chi-squared test for trend in proportions, positive trend, P=2.2×10⁻⁶), Validation set (n=103 samples, chi-squared test for trend in proportions, positive trend, P=4.5×10⁻⁰⁵).

FIG. 2—Phylogenetic tree and novel bacteria. A) Cultured fastidious anaerobes were isolated from urine and prostate and their genomes were decoded using Illumina and Nanopore DNA sequencing. Each bacterial strain was positioned on the phylogenetic tree as described in Methods. Bacteria with a known ID are highlighted with diamonds and a † symbol, while novel species are highlighted with diamonds and a * symbol (I: Varibaculum sp. nov., II: Fenollaria sp. nov., III: Peptoniphilus sp. nov. and IV: Porphyromonas sp. nov.) B) Genome representation for two of the novel species where the entire genomes could be constructed. Data on: I, Varibaculum sp. nov. isolate 39, 2.2 Mb, GC content, 53%; II, Fenollaria sp. nov. isolate 24, 1.6 Mb, GC content, 36%; III, Peptoniphilus sp. nov. isolate 23, 1.9 Mb, GC content 49%, and IV, Porphyromonas sp. nov. (isolate 6C, 2.2 Mb, GC content 56%) amongst other isolates are in FIG. 15 and Table 3, FIG. 8A-D. PVC: Planctomycetes, Verrucomicrobiae, Chlamydiae group.

FIG. 3—Presence and composition of urine and prostate tissue microbiota identify participants with a poorer prognosis. Analysis of 16S OTU sequence from urine sediments (A-C). A) Principal Coordinate Analysis (PCoA, Manhattan distance) of family level OTU data from urine sediments from 46 patients undergoing assessment for prostate cancer. Cluster 1 (black, upper left), Cluster 2 (yellow, upper right) and Cluster 3 (green, lower). Samples from patients that developed skeletal metastases are indicated with stars. B) Heatmap demonstrating a variety of bacterial genera selected to demonstrate differences across the 3 family level clusters. C) Kaplan-Meier analysis investigating metastasis free survival: cluster one (black, lower line); clusters two plus three (pink, upper line). (D-F) The presence of the following genera was used to partition sample sets: Fenollaria (including hits to Ezakiella due to closely related 16S sequences to Fenollaria), Peptoniphilus, Porphyromonas, Anaerococcus and Fusobacterium. These genera collectively constitute the anaerobic bacteria biomarker set (ABBS) in this manuscript. Data were from: (D) urine cell sediment fraction 16S sequencing; (E) RNA sequencing of urine exosome fraction; and (F) whole genome ICGC DNA sequence data from prostate cancer tissue. All “P” values are calculated using the log-rank test. Univariate cox proportional hazards models are summarised in FIG. 10. The hazard ratios (95% confidence intervals) are respectively as follows: 6.18 (95% Cl: 0.81-47.3), 6.10 (95% Cl: 1.34-27.26) and 2.07 (95% Cl: 1.04-4.15). A meta-analysis of these three models gave HR 2.93 (95% Cl: 1.43-6). A forest plot of this meta-analysis is available in FIG. 10.

FIG. 4—Predicted bacterial functions and metabolic pathways. Predicted proteins and metabolic pathways were determined with Prokka annotation and InterProScan analyses. The data shown highlight pathways across assembled isolates from urine and prostate secretions. Columns are ordered to highlight species isolated belonging to the ABBS genera (left ‘ABBS isolates’) vs bacteria that do not have sufficient evidence for contributing towards a poorer prognosis and were isolated from urine samples from patients with no evidence of cancer (right ‘Non-ABBS isolates’). The colour of each cell denotes the number of genes in an assembly that are estimated to contribute towards a particular function. The pathways in this heatmap were curated to demonstrate those with biological or therapeutic potential.

FIG. 5—Association of presence of bacterial aggregates with prostate cancer clinical grade. A) Scanning electron microscopy image of bacterial aggregates in urine cell sediment fraction, post-DRE. Magnification: 2,500×, scale bar indicates 10 μm. B) Fluorescence microscopy image of bacterial aggregates and human cell nuclei stained with 4′,6-diamidino-2-phenylindole (DAPI), scale bar indicates 10 μm. C) Association of presence of bacterial aggregates in post-DRE urine cell sediment and clinical category (P=0.006, log-rank test).

FIG. 6—Fluorescence microscopy images of cells in urine samples stained with DAPI. Example images of microorganisms associated with human cell nuclei (scale bar 10 μm). Microorganisms and human cell nuclei were stained with DAPI. Variation of focal depth demonstrated the presence of microorganisms within the eukaryotic cells.

FIG. 7—Comparison of Propionimicrobium lymphophilum isolated strains with reference strain DSM4903 and Cutibacterium acnes. The colour of the box surrounding a bacteria name (left) links to the map of that bacteria in the same colour shown on the circos plot (right). From the inside circus plot to outer: Propionimicrobium lymphophilum reference strain DSM4903 (light pink); Propionimicrobium lymphophilum isolate 4 (royal blue); Propionimicrobium lymphophilum isolate 9 (light blue); Propionimicrobium lymphophilum isolate 10 (yellow); Propionimicrobium lymphophilum isolate 3 (green); Propionimicrobium lymphophilum isolate 40 (orange); Cutibacterium acnes (pink).

FIG. 8—Bacteria isolated from urine and prostate secretion samples. Phylogenetic tree and colony morphology of isolated bacteria are presented. The panels of this Figure (FIG. 8 A to D) are an expansion of sections of FIG. 2. The coloured diamonds indicate characterised colonies corresponding to novel species ( [symbol] *) and those with known ID ( [symbol] †). Properties of the four novel species are as follows. Novel 1: Peptoniphilus sp. nov. isolate 23, (phyla Firmicutes, class Clostridia) isolated from urine sample of a high-risk prostate cancer patient. Colony appearance 2 mm slight yellow, grey tinge, glossy (A) Novel 2: Fenollaria sp. nov. isolates (24, 27A, 27B, 29, 35) belonging to genus Fenollaria, (phyla Firmicutes, class Clostridia) were isolated from a urine sample of a high-risk prostate cancer patient. Colony appearance 1-2 mm flat grey (A), a spore-forming pleomorphic species ranging in appearance from tiny <500 nm coccoid forms to larger filamentous forms (see FIG. 12 for further images). Novel 3: Varibaculum sp. nov. isolate 39, (phyla, class Actinobacteria) isolated from urine sample of a patient assessed for prostate cancer. A prostate biopsy tissue sample from this patient had atrophy and inflammation but there was no evidence of cancer. Colony appearance 1 mm, round grey and glossy, with α-haemolysis on brucella blood agar plates (C). Novel 4: Porphyromonas sp. nov., isolate 6C, isolated from prostate secretion fluid from prostatectomy specimen from a patient with intermediate risk prostate cancer. Colony appearance, small 1 mm, glossy grey pigmented (D), pleomorphic species, coccoid and filamentous forms.

FIG. 9—Partitioning of bacteria between Principle Coordinate Analysis clusters. (A-C) Principal Coordinates Analysis of bacterial communities from 16S sequencing of urine sediment fraction (Manhattan distance, Family level). The first three principal coordinates are shown along with the percentage of variation of the dataset that the axis demonstrates. (D) Plots of relative abundance (y-axis) for various bacterial genera. Differences are investigated between the poor prognosis cluster (Cluster 1) and the rest of the dataset (Clusters 2+3). P=Unpaired Wilcoxon Rank Sum test with q=Benjamini-Hochberg correction for multiple comparisons.

FIG. 10—Forest plot showing a meta-analysis of univariate cox proportional hazards models demonstrating hazard ratio (HR) for three technologies (16S sequencing, ICGC whole genome sequencing and Urine RNA shotgun sequencing) along with 95% confidence intervals. A) Clinical endpoints and R packages used were as discussed in methods. Heterogeneity between studies appears low (Higgin's & Thompson's I2=0%, chi-squared χ2=1.55, but not significant at P=0.46). The meta-analysis was determined using a random effects model. B) Forest plot showing multivariate analysis for the ICGC cohort with covariates including prostate specific antigen (PSA) blood test at the time of radical prostatectomy (RP), age at diagnosis, tumour size at diagnosis, Gleason Grade and ABBS detection status. (16S sequencing, ICGC whole genome sequencing and Urine RNA shotgun sequencing) along with 95% confidence intervals. Clinical endpoints and R packages used were as discussed in methods. Heterogeneity between studies appears low (Higgin's & Thompson's I²=13%, chi-squared χ2=2.3, but not significant at P=0.32). The meta-analysis was determined using a random effects model.

FIG. 11—Co-occurrence of bacteria genera identified in urine sediment from men with high risk, advanced/metastatic prostate cancer. Co-occurrence plot prepared using MEGAN Community Edition (version 6.8.12). Bacterial genera assigned from the 16S sequencing dataset as described in methods and results were included in the analyses. Samples included were from men with high risk and advanced/metastatic prostate cancer (n=14), both the V1-V3 and V3-V5 16S sequencing data analyses were included. The minimum percentage probability with which two classes co-occur in samples is 75%. Circles are coloured by phylum and named at the level of genus. The black straight lines joining circles indicate co-occurrence. Light peach/orange=Firmicutes, includes Fenollaria sp.; Peptoniphilus sp., Anerococcus sp, Finegoldia sp. Green=Bacteroidetes. Purple=Proteobacteria. The orange diamond [symbol] * indicates that novel species were isolated belonging the genera. The blue diamond [symbol] † indicates that we isolated and cultured several species belonging to these genera (details in FIG. 2, FIG. 8 and FIG. 11).

FIG. 12—Images of Fenollaria sp. nov. displaying pleomorphic growth and sporulation. Images on the left are differential interference contrast microscopy images of Fenollaria sp. nov. after growth under anaerobic conditions in PY broth and images on the right are corresponding fluorescent microscopy images of Fenollaria sp. nov. stained with DAPI, scale bar indicates 10 μm.

FIG. 13—An overviewflow chart of the analyses in this study highlighting the identification of ABBS and novel species.

FIG. 14—Association of presence of bacteria in urine sediment with prostate cancer clinical grade, stage and PSA. Data are presented in A-C as percentage positive for bacteria/microorganisms. 0+0=PSA normal for age and no cancer detected on biopsy; Gleason score 3+3, 3+4, 4+3, 4+4>8; A=advanced prostate cancer. (A, B) association of presence of bacteria and prostate cancer Gleason Score, significant positive trend across increased cancer grade, Discovery set (χ²-test, 15.01, df=1, p=1.1×10-4) and Validation set (χ²-test, 9.11, df=1, p=2.5×10-3). (C) significant association of samples positive for bacteria and clinical T stage (χ²-test, 18.01, df=1, p=2.2×10-5) (D) samples positive for bacteria were from participants with significantly higher PSA results (p=1.7×10-8, Mann-Whitney U test). There was also a slight increase in the age of those patients that have bacteria present (median 70 vs 67, Mann-Whitney U test: P=0.048).

FIG. 15—Bacterial species cultured from urine samples and prostate secretions. Table documents culture morphology and genomic characteristics for each of the isolates including colony appearance, novelty, average sequencing coverage genome size, GC content, number of contigs, estimated number of genes (calculated using the Prokka tool, version 1.13.3). Average sequencing coverage was calculated with BBMap version 37.28. All isolates were characterised by Illumina sequencing. Strains 24, 25 and 39 were subject to additional long read sequencing using the Oxford Nanopore MinION.

FIG. 16—Detection of bacterial species in urine samples using qPCR assays. Data presented as percentage of samples positive for bacteria genera or species across the clinical categories. Significant associations (p<0.05) for the presence of bacteria in urine with increasing prostate cancer clinical grade and risk groups was observed for the bacteria. Bacteria from Firmicutes phyla of bacteria (FIGS. 16A and 16B; 16E and 16F), and Bacteroidota phyla (FIGS. 16C and 16D).

FIG. 17—Detection of bacterial species in prostate tumor whole genome sequencing samples. SEPATH [63] was ran on N=2,176 prostate tumour whole genome sequencing samples from the pan-prostate cancer group (http://panprostate.org). Participants were filtered to those containing followup data (N=818). Participants with evidence for at least one ABBS genera (N=146, blue, as determined by a minimum of 10 taxonomically classified sequencing reads by Kraken for at least one of the genera: Fenollaria, Ezakiella, Peptoniphilus, Porphyromonas, Anaerococcus, Fusobacterium) were compared to those that had no evidence of ABBS genera (N=672, red). There was a statistically significant difference between the groups in terms of relapse-free survival (P=0.023, log-rank test), with the ABBS group demonstrating a poorer prognosis.

### DETAILED DESCRIPTION OF THE INVENTION

For patients with suspected prostate cancer the risk of over-diagnosis and a lack of prognostic indicators leads to difficulties in clinical decision making where radical surgery or radiotherapy are treatment options for potentially aggressive disease. In this study several separate lines of evidence supportthe role of bacteria as a prognostic marker. First, we demonstrated a significant correlation between risk of prostate cancer progression and the presence of bacteria as determined by the fluorescence microscopic detection of DNA, both in discovery (n=215 patients, P=2.2×10⁻⁶) and validation (n=103, P=4.5×10⁻⁰⁵) datasets. Secondly, Principal Coordinate Analysis of 16S OTU data identified a distinct cluster of patients with higher incidence of metastatic disease. This observation allowed the development of the Anaerobic Bacteria Biomarker Set (ABBS) consisting of 5 genera of strictly anaerobic bacteria (Table 4). Thirdly, application of ABBS to RNAseq libraries prepared from the exosome fraction of urine and to OTU data from the urine sedimentary fraction gave results indicative of the importance of this bacterial group in predicting clinical outcome, although the datasets examined were of modest size and not independent. Next through interrogation of a much larger dataset of Whole Genome DNA Sequence data from prostate cancer (n=204) we were able to confirm the ability of ABBS to predict prostate cancer progression. The combined hazard ratio for all studies using ABBS was: HR 2.93, 95% Cl: 1.43-6. Taken together these studies provided a strong case for a role of bacteria present in the prostate-urine reflux loop in predicting the clinical progression of prostate cancer.

These observations led to the hypothesis that the bacteria comprising ABBS, associated with aggressive disease, can prosecute a broad range of biological actions that have the potential to co-ordinate and cause cancer progression. Previously inflammation has been strongly linked with progression of PCa [25,26,27], and it has been documented for several cancers that bacterial infection can cause inflammation [36,40]. A review published in 2019 [25], describes the association of the microbiota with prostate pathologies but concluded that major difficulties remain: sampling contamination, obtaining effective control tissue and, with the advent of new sequencing technologies, classifying the often-novel bacteria involved. In this study we used protocols to ensure minimal contamination [70] during the generation of OTU data. We have also grown and whole genome sequenced bacteria leading to the classification of several novel organisms, based on ribosomal protein genes as described by Hug [41].

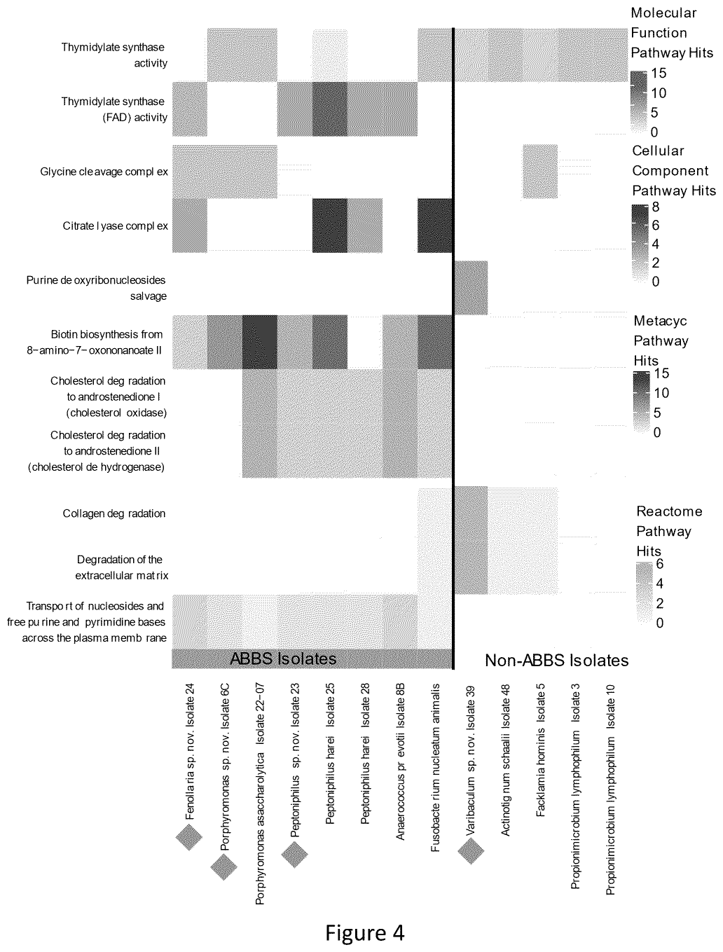

The new bacteria were commonly found in urine. This improved characterisation of urine bacteria and development of ABBS allowed us to map the metabolic ability of bacteria associated with prostate cancer progression. Interestingly, we did not find any obvious pro-inflammatory metabolism specific to the ABBS. In a previous study of n=30 patients [42] detected faecal microbiota changes in patients receiving oral androgen receptor axis-targeted therapies compared to control groups, with an increase in bacteria including Akkermansia sp. [76]. In this study we found that several species belonging to the genera associated with poor clinical outcome have predicted components of metabolic pathways that can convert cholesterol to androstenedione (FIG. 4), which if present would negate the impact of the androgen receptor axis-targeted prostate cancer drug abiraterone.

Recent studies have undertaken comprehensive analyses of microbiomes [43,44] and viromes [45] associated with human cancers. A consistent observation is that microbiomes present in cancer tissue or blood can act as diagnostics markers across multiple cancer types [43,44]. Here we showthatthe microbiome in urine and in cancer tissue can act as a prognostic marker for clinical outcome in prostate cancer. Analyses of published datasets [43,44] indicated that the ABBS bacteria genera we found to be associated with poor prognosis in prostate cancer were also present in other cancer tissue types, hence their relevance in determining aggression may extend beyond prostate cancer. One of these studies [43] additionally reported the association of metabolic functions encoded by bacteria and clinical features including tumour subtypes, smoking status, and the response to immunotherapy. They found that bacteria associated with cancer were intracellular [43], in agreement with observations in this study.

In some embodiments of the invention, the cancer type can be selected from the list: Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Central Nervous System (Brain Cancer), Basal Cell Carcinoma, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors (Lung Cancer), Burkitt Lymphoma—see Non-Hodgkin Lymphoma, Carcinoid Tumor (Gastrointestinal), Cardiac (Heart) Tumors, Medulloblastoma and Other CNS Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Embryonal Tumors, Medulloblastoma, Endometrial Cancer, Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Heart Tumors, Hepatocellular (Liver) Cancer, Histiocytosis, Langerhans Cell, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Melanoma, Intraocular (Eye), Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer) Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, Acute (AML), Myeloproliferative Neoplasms, Chronic Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma, Childhood Rhabdomyosarcoma (Soft Tissue Sarcoma), Childhood Vascular Tumors (Soft Tissue Sarcoma), Ewing Sarcoma (Bone Cancer), Osteosarcoma (Bone Cancer), Soft Tissue Sarcoma, Uterine Sarcoma, Sezary Syndrome, Skin Cancer, Small Cell Lung Cancer, Small Intestine Cancer, Squamous Cell Carcinoma, Squamous Neck Cancer with Occult Primary, Metastatic (Head and Neck Cancer), Stomach (Gastric) Cancer, T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Ureter and Renal Pelvis, Transitional Cell Cancer (Kidney (Renal Cell) Cancer, Urethral Cancer, Uterine Cancer, Vaginal Cancer, Vascular Tumors, Vulvar Cancer, Wilms Tumor, Myxofibrosarcoma, Leiomyosarcoma, Pleomorphic Sarcoma and Hepatopancreatobiliary cancer.

In a preferred embodiment of the invention, the cancer type is prostate cancer.

To understand the urine and prostate microbiome, we carried out analyses of the bacteria present using 16S amplicon DNA sequencing and anaerobic culture leading to the identification of four novel anaerobic bacteria species and to the characterisation of a set of five bacteria genera (ABBS) associated with aggressive prostate cancer. The use of ABBS predicted rapid development of aggressive disease in three datasets using three distinct technologies with an overall Hazard Ratio (HR) of 2.93, 95% Cl: 1.43-6. The data presented in the current study provide a strong basis for future investigation of carcinogenic mechanisms. Our data are more consistent with a model where a group of strict anaerobic bacteria act together, making the situation more complicated than the established association between H. pylori and gastric cancer. Initial analyses of pathways predicted from bacteria whole genome DNA sequence data have indicated possible mechanisms that can be tested in future studies, including the production of testosterone precursors that may drive cancer growth.

### Active Surveillance

Active surveillance (AS) is a means of disease-management for men with localised PCa with the intent to intervene if the disease progresses. AS is offered as an option to men whose prostate cancer is thought to have a low risk of causing harm in the absence of treatment. It is a chance to delay or avoid aggressive treatment such as radiotherapy or surgery, and the associated morbidities of these treatments. Entry criteria for men to go on active surveillance varies widely and can include men with Low risk and Intermediate risk prostate cancer.

Patients on AS are currently monitored by a wide range of means that include, for example, PSA monitoring, biopsy and repeat biopsy and MP-MRI. The timing of repeat biopsies, PSA testing and MP-MRI varies with the hospital, and a widely accepted method for monitoring men on AS has not yet been achieved.

In some embodiments, active surveillance comprises assessment of a patient by PSA monitoring, biopsy and repeat biopsy and/or imaging techniques such as MRI, for example MP-MRI. In some embodiments, active surveillance comprises assessment of a patient by any means appropriate for diagnosing or prognosing prostate cancer.

In some embodiments of the invention, active surveillance comprises assessment of a patient at least every 1 month, 2 months, 3 months, 4 months, 5 months, 6 months, 7 months, 8 months, 9 months, 10 months, 11 months or 12 months.

In some embodiments of the invention, active surveillance comprises assessment of a patient at least every 1 year, 2 years, 3 years, 4 years or 5 or more years.

In some embodiments of the invention the presence of ABBS bacteria will be used alone or in conjunction with other means of testing to improve shared decision making with the multi-disciplinary team and the patient. The presence or absence of ABBS bacteria could be used to decide whether radical intervention is necessary, to determine whether anti-microbial therapy is required or to decide the optimal time between re-monitoring by, for example, biopsy, PSA testing, or MP-MRI.

### Biological Samples

In the present invention, the biological sample may be a urine sample, a semen sample, a prostatic exudate sample, or any sample containing macromolecules or cells originating in the prostate, a whole blood sample, a serum sample, saliva, or a biopsy (such as a prostate tissue sample or a tumour sample), although urine samples are particularly useful. The method may include a step of obtaining or providing the biological sample, or alternatively the sample may have already been obtained from a patient, for example in ex vivo methods.

Biological samples obtained from a patient can be stored until needed. Suitable storage methods include freezing immediately, within 2 hours or up to two weeks after sample collection. Maintenance at −80° C. can be used for long-term storage. Preservative may be added, or the urine collected in a tube containing preservative. Urine plus preservative such as Norgen urine preservative, can be stored between room temperature and −80° C.

Methods of the invention may comprise steps carried out on biological samples. The biological sample that is analysed may be a urine sample, a semen sample, a prostatic exudate sample, or any sample containing macromolecules or cells originating in the prostate, a whole blood sample, a serum sample, saliva, or a biopsy (such as a prostate tissue sample or a tumour sample). Most commonly for prostate cancer the biological sample is from a prostate biopsy, prostatectomy or TURP. The method may include a step of obtaining or providing the biological sample, or alternatively the sample may have already been obtained from a patient, for example in ex vivo methods. The samples are considered to be representative of the expression status of the relevant genes in the potentially cancerous prostate tissue, or other cells within the prostate, or microvesicles produced by cells within the prostate or blood or immune system. Alternatively, the samples can be considered to be representative of the potentially cancerous microenvironment of the prostate, comprising gene expression or methylation and protein expression. Hence the methods of the present invention may use quantitative data on DNA, RNA, methylation and proteins produced by cells within the prostate and/or the blood system and/or bone marrow in response to cancer, to determine the presence or absence of prostate cancer.

The methods of the invention may be carried out on one test sample from a patient. Alternatively, a plurality of test samples may be taken from a patient, for example at least 2, 3, 4 or 5 samples. Each sample may be subjected to a separate analysis using a method of the invention, or alternatively multiple samples from a single patient undergoing diagnosis could be included in the method.

The sample may be processed prior to determining the expression status of the biomarkers. The sample may be subject to enrichment (for example to increase the concentration of the biomarkers being quantified), centrifugation or dilution. In other embodiments, the samples do not undergo any pre-processing and are used unprocessed (such as whole urine).

In some embodiments of the invention, the biological sample may be fractionated or enriched for DNA prior to detection and quantification (i.e. measurement). The step of fractionation or enrichment can be any suitable pre-processing method step to increase the concentration of DNA in the sample or select for specific sources of DNA such as cells or extracellular vesicles. For example, the steps of fractionation and/or enrichment may comprise centrifugation and/or filtration to remove cells or unwanted analytes from the sample, or to increase the concentration of bacteria in a urine fraction. Methods of the invention may include a step of amplification to increase the amount of DNA that is detected and quantified. Methods of amplification include PCR amplification. Such methods may be used to enrich the sample for any bacteria of interest.

In some embodiments of the invention, the biological sample may be fractionated or enriched for RNA prior to detection and quantification (i.e. measurement). The step of fractionation or enrichment can be any suitable pre-processing method step to increase the concentration of RNA in the sample or select for specific sources of RNA such as cells or extracellular vesicles. For example, the steps of fractionation and/or enrichment may comprise centrifugation and/or filtration to remove cells or unwanted analytes from the sample, or to increase the concentration of bacteria in a urine fraction. Methods of the invention may include a step of amplification to increase the amount of RNA that is detected and quantified. Methods of amplification include PCR amplification. Such methods may be used to enrich the sample for any bacteria of interest.

In some embodiments of the invention, the biological sample may be fractionated or enriched for bacterial protein prior to detection and quantification (i.e. measurement). The step of fractionation or enrichment can be any suitable pre-processing method step to increase the concentration of bacterial protein in the sample or select for specific sources of bacterial protein such as cells or extracellular vesicles. For example, the steps of fractionation and/or enrichment may comprise centrifugation and/or filtration to remove cells or unwanted analytes from the sample, or to increase the concentration of bacteria in a urine fraction. Methods of the invention may include a step of amplification to increase the amount of bacterial protein that is detected and quantified. Such methods may be used to enrich the sample for any bacteria of interest.

Bacterial nucleic acids and/or proteins may be extracted from the biological sample. This can be achieved by a number of suitable methods. For example, extraction may involve separating the bacterial nucleic acids and/or proteins from the biological sample. Methods include chemical extraction and solid-phase extraction (for example on silica columns). Preferred methods of nucleic acid extraction include the use of a silica column. Methods comprise lysing cells or vesicles (if required), addition of a binding solution, centrifugation in a spin column to force the binding solution through a silica gel membrane, optional washing to remove further impurities, and elution of the bacterial nucleic acid and/or protein. Commercial kits are available for such methods, for example from Qiagen or Exigon.

If bacterial nucleic acids and/or proteins are extracted from a sample, the extracted solution may require enrichment to increase the relative abundance of bacterial nucleic acids and/or proteins transcripts in the sample.

The methods of the invention may be carried out on one test sample from a patient. Alternatively, a plurality of test samples may be taken from a patient, for example at least 2, at least 3, at least 4 or at least 5 samples. Each sample may be subjected to a single assay to quantify one of the biomarker panel members, or alternatively a sample may be tested for all of the bacteria being detected.

### Methods of the Invention

### Patient Recruitment and Collection of Urine Samples

Urine samples were collected post-DRE from patients undergoing assessment for prostate cancer or haematuria at the Norfolk and Norwich University Hospital. Ethical approval was obtained from the local research ethics committee (12/EE/0058). Patients were categorised as either normal PSA range for age, raised PSA negative biopsy/raised PSA no biopsy, atypia/High grade prostatic intraepithelial neoplasia (HG-PIN), or cancer risk group (low, intermediate, high) being assigned according to D'Amico criteria based on Gleason score, PSA and clinical stage [5]. Patients were assigned as having advanced disease when PSA>100 ng, with presence of metastases on bone scan or on MRI scan. A summary of the clinical characteristics are shown in Tables 11-14. Following DRE the first 10-25 ml of urine was collected into a sterile 30 ml Universal collection tube (Bibby Sterilin, UK). 300 μl of urine were pipetted onto urine dipsticktest strips (Roche Cobas Combur, Roche Diagnostics, Germany) and the remaining sample was centrifuged at 1200×g for 5 min at 6° C. The urine supernatant was processed and nucleic acids extracted as described by Connell et al [34]. The cell sediment was gently re-suspended in ˜100-200 μl of the supernatant and 80% of the cell pellet volume was aliquoted into PCR grade Eppendorf tubes (Eppendorf, Germany), snap frozen on dry-ice and stored at −80° C. for the later extraction of DNA. The remaining 20% of the urine cell pellet was pipetted onto 2 glass Histobond/SuperFrost slides (Tekdon Inc., USA) and incubated at 4° C. for 30 min to allow cells to bind to the slide. The bulk of the liquid fraction was removed and 50 μl SprayFix cytological fixative (GCC diagnostics, UK) was added to fix material bound to the slide. The slides were allowed to dry at room temperature for 1 hour before storage or preparation for imaging.

### Detection of DNA and Bacteria by DAPI Staining and Microscopy

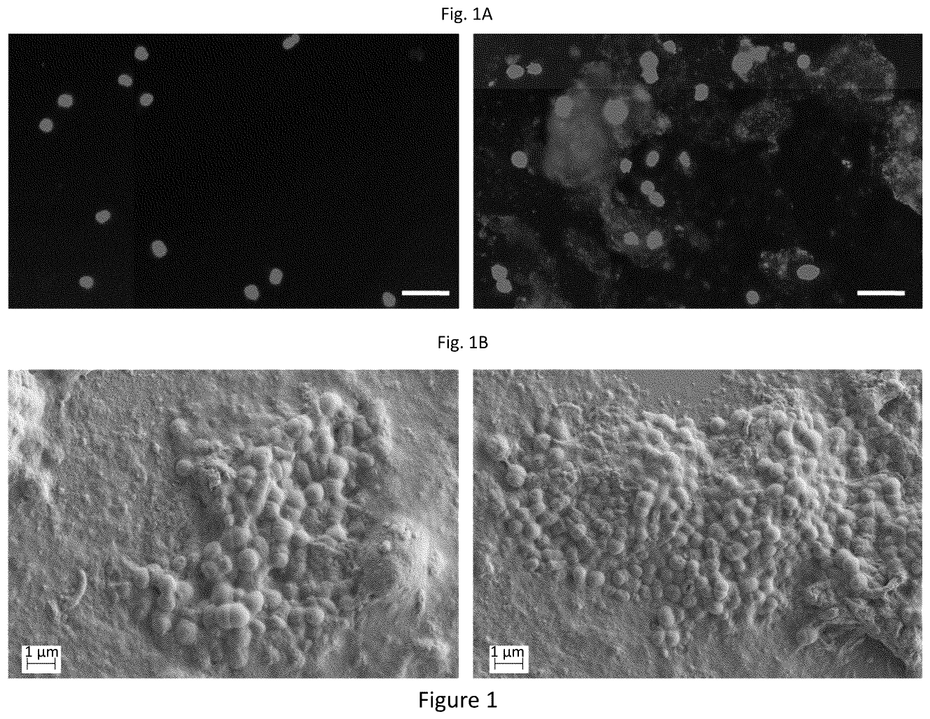

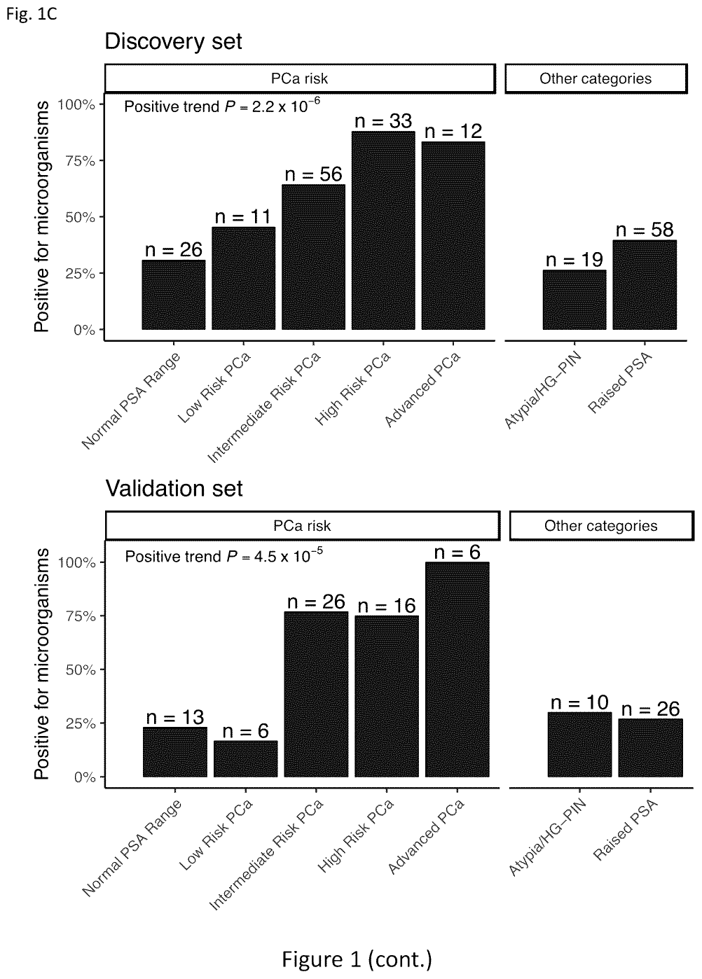

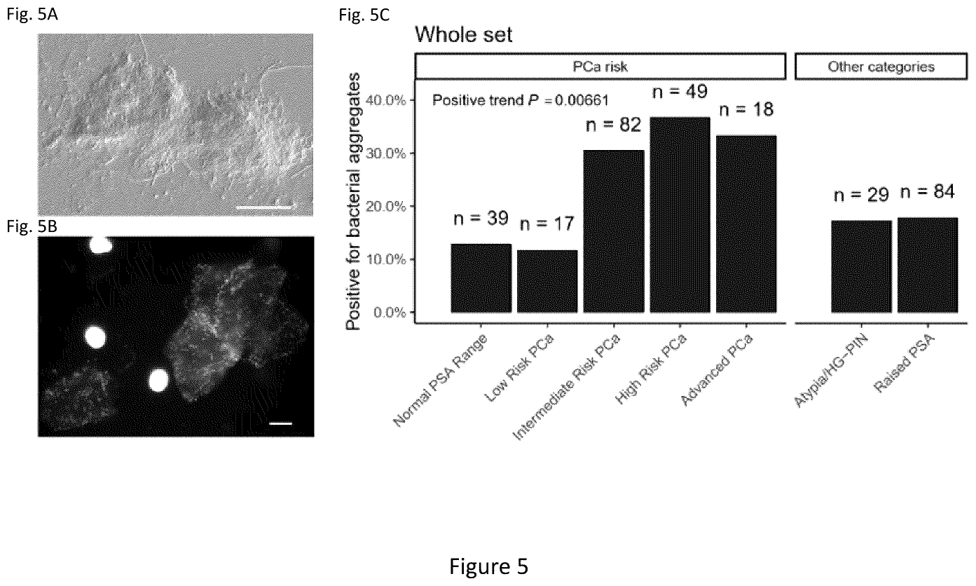

Slides containing bound urine cell sediment material were soaked in 70% (v/v) ethanol for 30 min and dried at room temperature. Glass coverslips (18×18 mm, Menzel-Glasser, Germany) were mounted onto the slides with 14 μl Vectashield® plus DAPI diluted 1:4 with Vectashield® mounting media (Vector Laboratories Inc., USA) and the slides stored at 4° C. for at least 30 min prior to fluorescence microscopy imaging using a Leica Ariol 6000B microscope. Slides were scanned with Leica 40× Fluotar objective, fixed exposure 3 colour fluorescence detection with Z-stack settings (7 images at 1.2 μm spacing) for 460 fields of view (FOV). Background DAPI staining indicating the presence of bacterial cell DNA was considered to be present if detected in equivalent to at least >10 full field of view from 460 FOVs screened, and also only if the DAPI staining of bacterial cell DNA was appropriate within the range for the size and morphology of bacteria. Scanning electron microscopy was used on seven samples to further investigate morphological appearances and to obtain accurate size measurements. Slides were coated with 5 nm gold and imaged using a Zeiss Gemini Supra 55 VP SEM and FEI Nova NanoSEM 450 scanning electron microscope. The detection of the morphology and size of the bacteria using SEM (example images in FIG. 1) revealed that the bacteria were pleomorphic, and included visible cocci, diplococci, pleomorphic rods, filament and bacterial aggregates. The cocci sizes ranged from 350-718 nm (mean±SD: 529±89 nm), the diplococci ranged from 538-961 nm (mean±SD: 728±121 nm), the pleomorphic rods width ranged from 194-611 and length ranged from 680-1705 (mean±SD width: 385+128, length: 1127±281 nm). The bacterial filamentous forms were often observed in large bacterial aggregates (aggregate size 50-60 μm, FIG. 5).

DNA Extraction from Urine Sediments and 16S Amplicon DNA Sequencing

DNA extraction from urine sediment was similar to the protocol of Yu and Morrison, 2004 [46] with repeated bead-beating extraction to maximise recovery of bacterial DNA. Bacteria present were identified by amplification and sequencing of the bacterial 16S rRNA [47]. The primer pairs selected were to the hypervariable regions V1-V3 and V3-V5. Primer sequences were V1-V3: V1F/27F: AGAGTTTGATCMTGGCTCAG (SEQ ID NO:21), V3R/534R: ATTACCGCGGCTGCTGG (SEQ ID NO:22), and V3-V5: V3F/357F: CCTACGGGAGGCAGCAG (SEQ ID NO:23), V5R/926R: CCGTCAATTCMTTTRAGT (SEQ ID NO:24). The PCR reactions were carried out with Accuprime PCR reagents (Invitrogen, UK), primers at 200 nM, with 15 ng DNA template added in a total volume of 25 μl. All of the steps outlined by Salter et al. (2014) [70] including the controls mentioned were incorporated to minimise the possibility of and to check for contamination Controls included no template controls, elution buffer controls and blank bead-beating extraction samples. PCR cycling conditions were: 94° C. 60 sec, followed by 25-30 cycles of 94° C. 30 sec, 57° C. or 50° C. for 10 sec, and 68° C. for 40 sec. 57° C. was used for V1-V3 and 50° C. for V3-V5 primers. Individual barcodes were added to the samples according to a 2-step PCR assay and samples were subject to by 454 pyrosequencing with titanium chemistry, (Beckman Coulter Genomics Washington USA). Mycoplasma hyorhinis was used as a positive control for 16S PCR.

Amplicon Sequencing analysis

Flow files were generated from the sff files provided by the 454 sequence data provider, using process_sff.py part of QIIME package v1.9.1-2050604. Filtering and denoising of the 454 data was performed by FlowClus v1.1 [48]. Chimera detection was performed by Vsearch v2.4.3 with default parameters during FlowClus pipeline. Vsearch classified chimeric sequences were removed from further analysis. Resulting sequences from three different pools for each primer pair (V1-V3 and V3-V5) were combined in to one dataset for each primer pair. The operational taxonomic units (OTUs) were selected de novo with Uclust [49] using pick_otus.py script from QIIME with similarity 0.99. Most abundant sequences in each cluster were used a representative sequence for OTUs. Representative OTUs were searched against NCBI-nt database (dated 1 Apr. 2017) using blastn program of BLAST+v2.6.0 (parameters: −task ‘blastn’-evalue 1E-10) to remove human contamination. OTUs and respective clusters matching to human with 97 percent identity or higher were removed from downstream analysis. Taxonomy assignments for OTUs were performed using RDP classifier [50] with SILVA release 132 ribosomal RNA database [51] as the reference. Biom files were created using resulting data as described in QIIME with make_otu_table.py. V1V3 and V3V5 data for samples with both datasets were combined to make one table with both abundances for each sample. This combined table is used to estimated alpha and beta diversity analysis using phyloseq v1.24.0 [52] package in R v3.5.0. In addition, various plot and heatmaps were generated in R using following packages ComplexHeatmap v1.18.0, survival v2.42-3, survminer v0.4.2, ggrepel v0.8.0, ggplot2 v 2.2.1, RColorBrewer v1.1-2 and reshape2 v1.4.3.

### Harvest of Prostate Biopsies and Secretion

Fresh prostate needle biopsies and prostate secretions were harvested within 20 min from whole intact prostate specimens post-prostatectomy while still in theatre using sterile equipment. The prostate was massaged and compressed to expel prostate secretions, which were collected into cryovials. Each prostate yielded around 100-400 μl of secretion. Secretion and tissue samples were transferred immediately to the laboratory for rapid processing and DNA extraction or snap frozen on dry ice and transferred to −80° C. freezer.

### Anaerobic Culturing of Bacteria

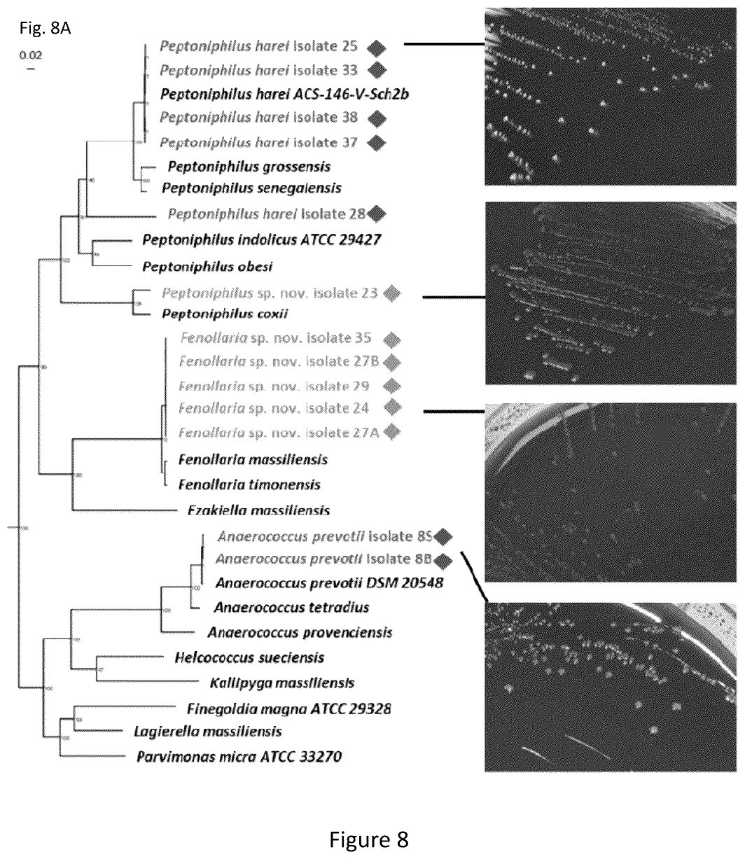

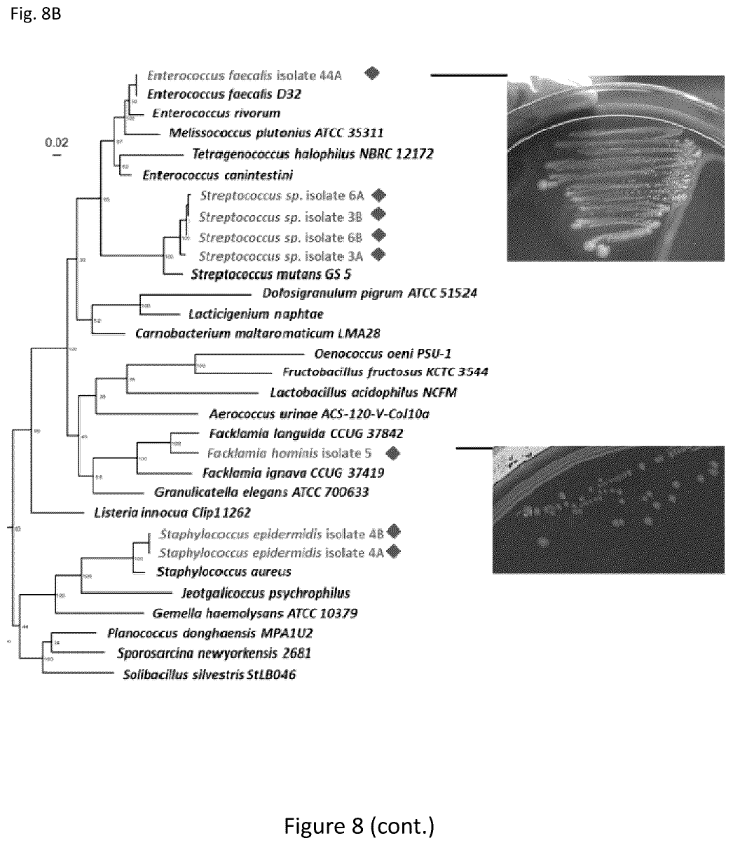

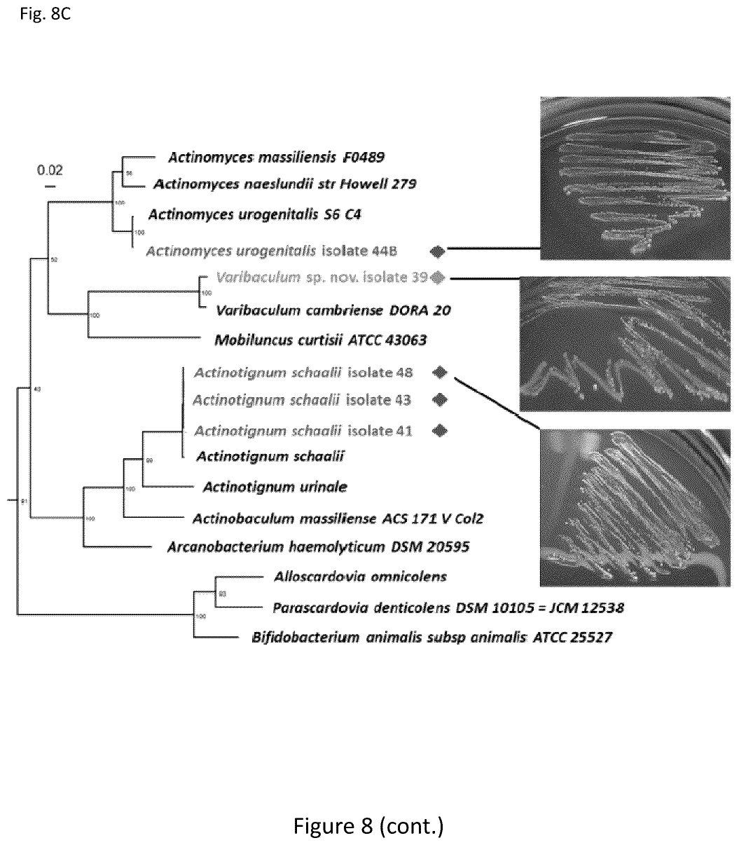

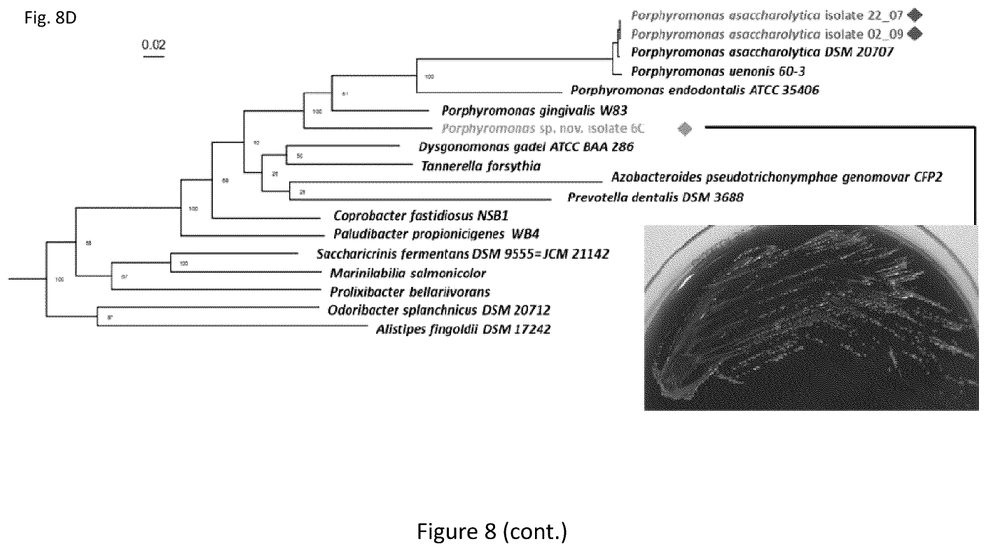

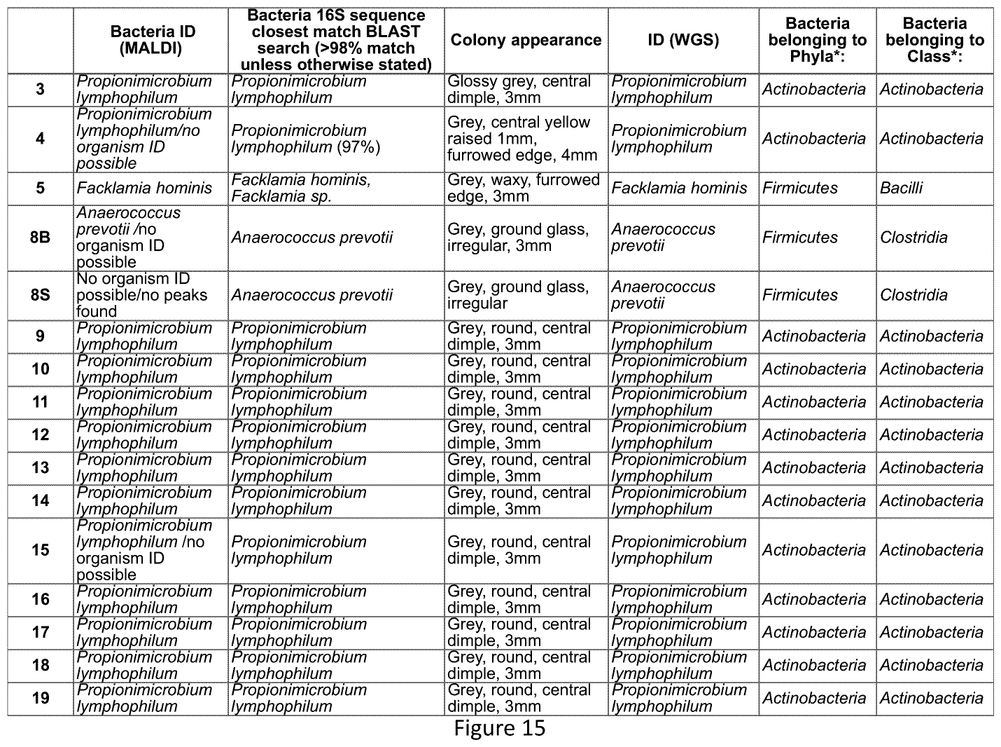

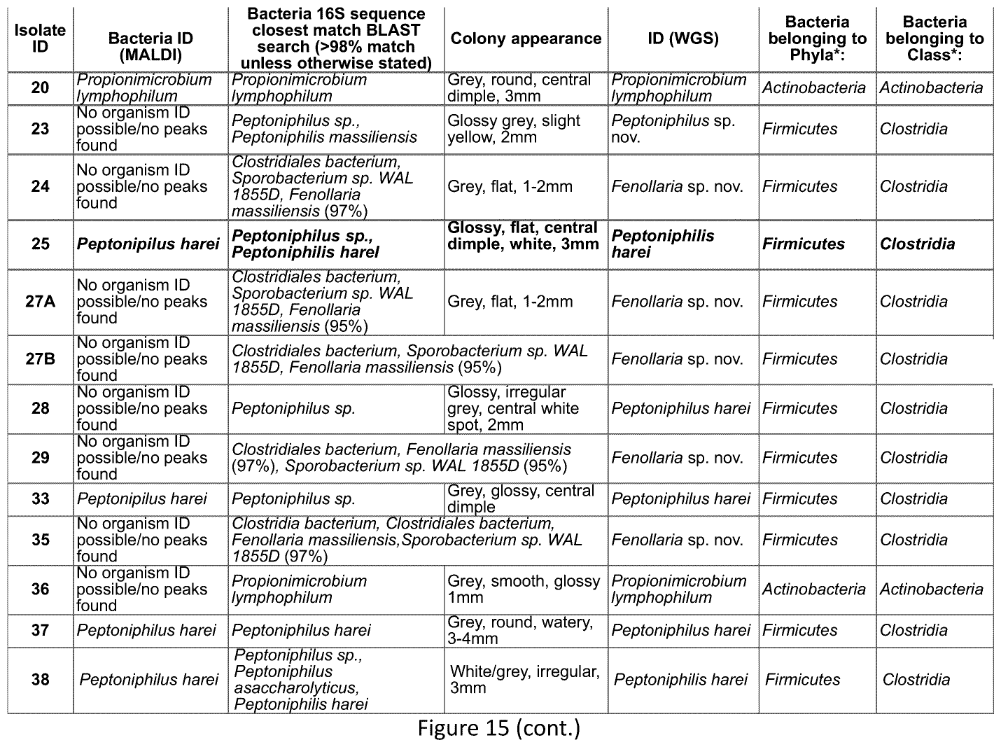

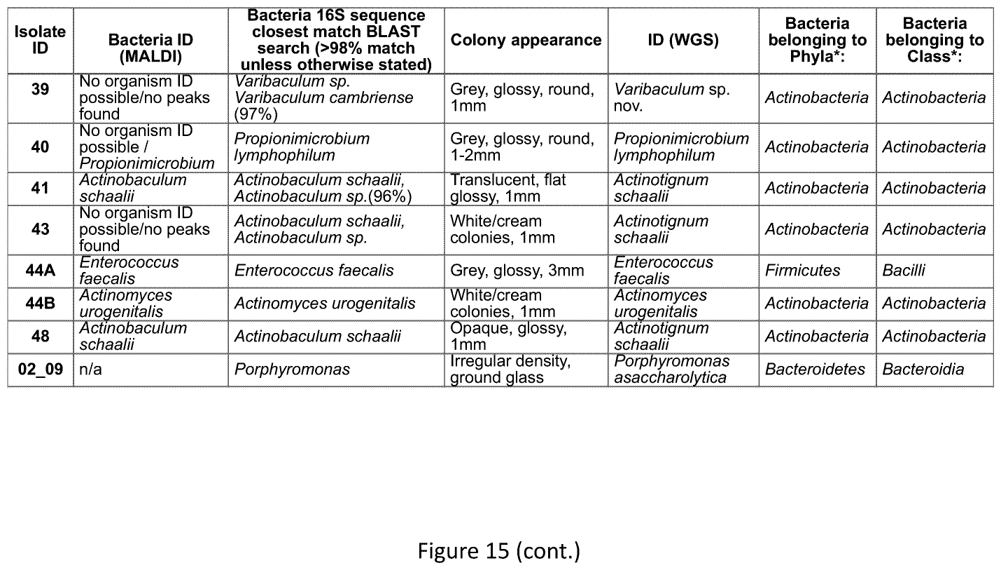

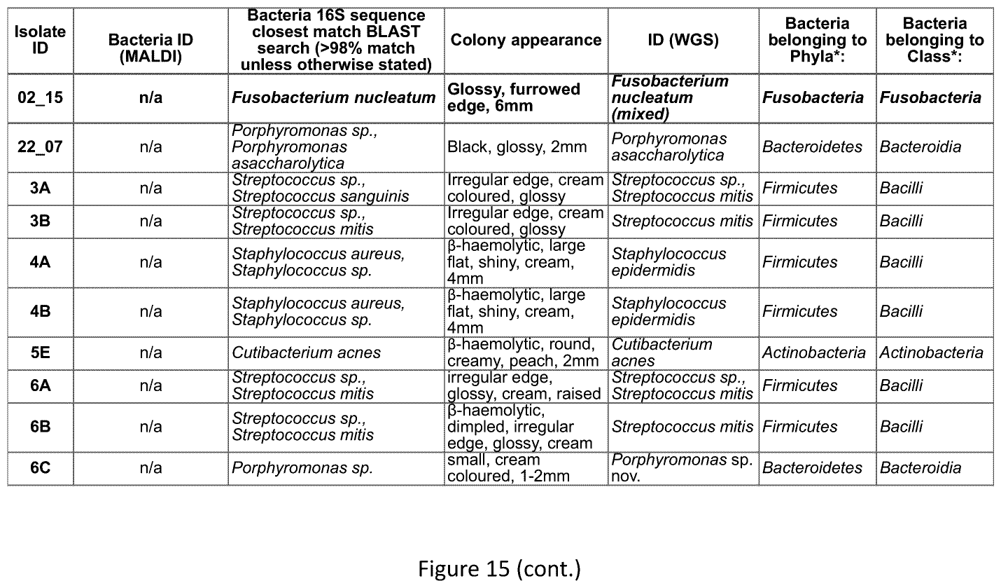

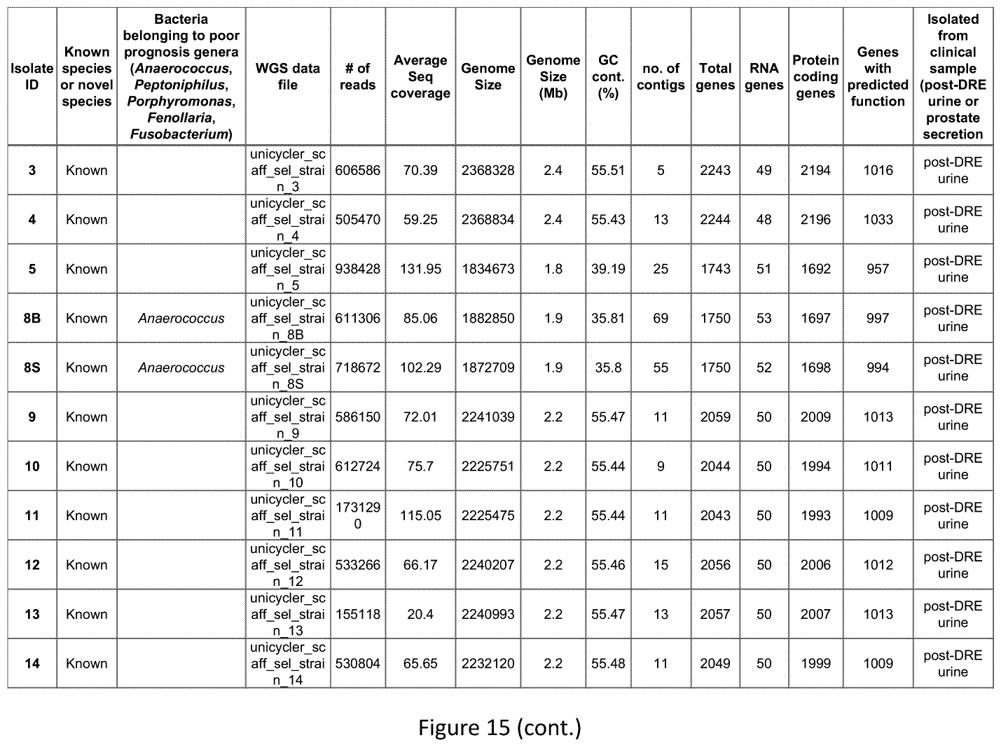

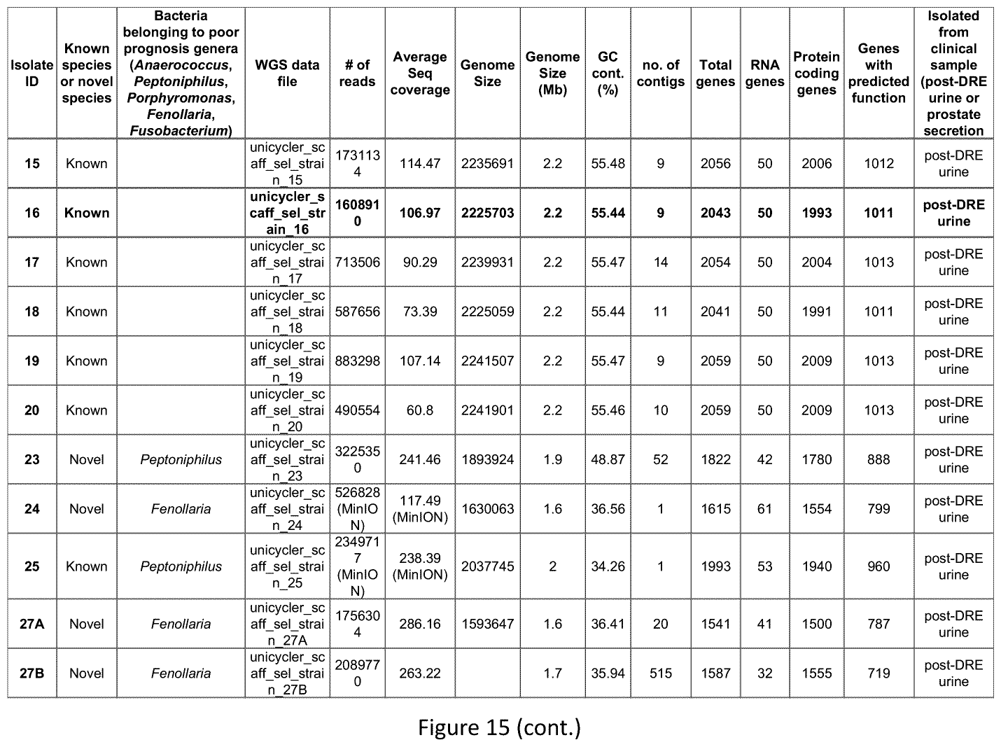

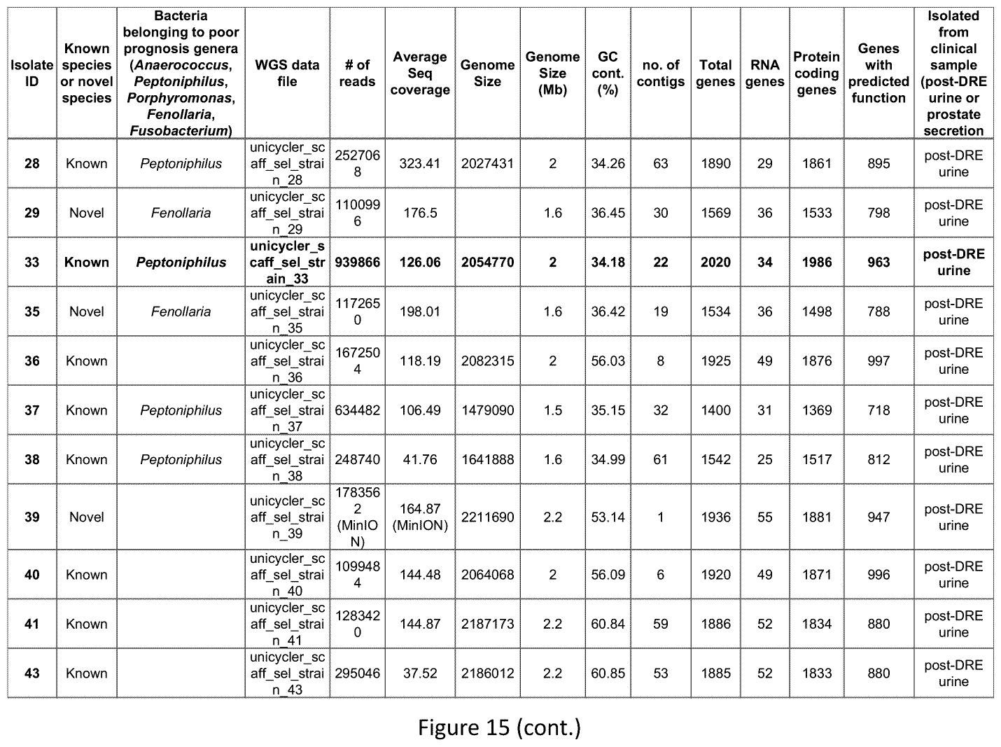

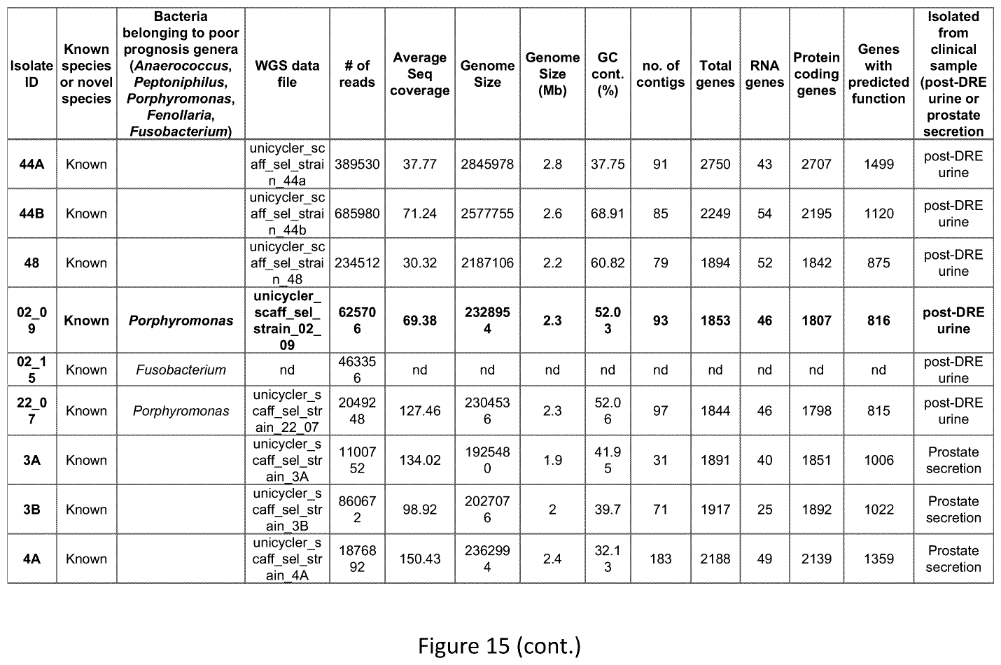

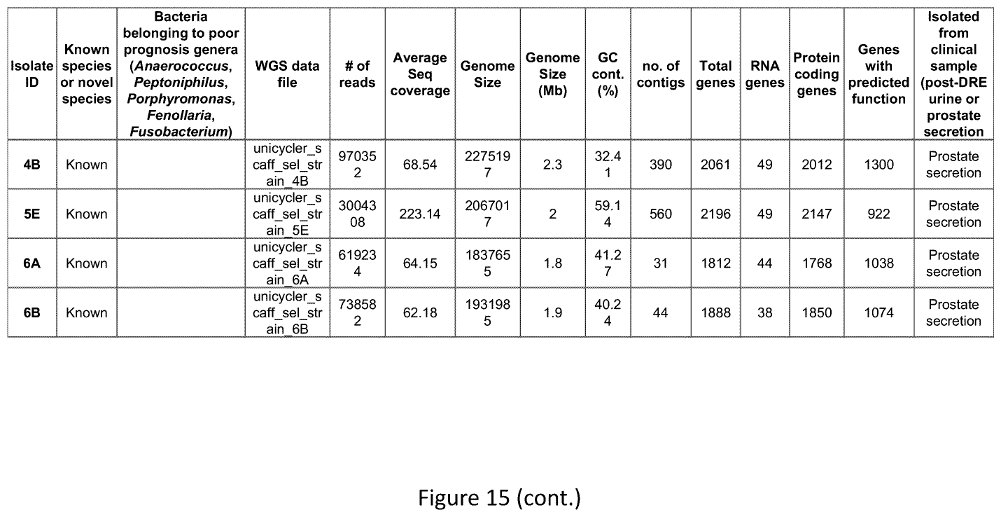

Samples were transferred to a microbiological cabinet supplied with anaerobic gas mixture supply: 5% Hydrogen, 10% CO₂, and 85% Nitrogen at 37° C. Samples taken from urine sediment or prostate secretions were inoculated into pre-reduced PY broth or Brucella blood agar plates with 5% sheep blood and vitamin K1/hemin supplementation (Beckton Dickinson GmbH Heidelberg, Germany) and cultured under anaerobic conditions for up to 7 days. Distinct colonies of each morphology present were picked under anaerobic conditions and cultures prepared for DNA extraction and preparation of frozen stocks. Colony morphology observations are as described in FIG. 8 and FIG. 15. MALDI analysis was via the Bruker MALDI Biotyper, server version 4.1.40 (SIRO) 325 according to manufacturer's instructions. Preparation of fastidious anaerobes onto slides for staining and fluorescence microscopy was as described above for urine sediment fractions.

Quantitative Polymerase Chain Reaction (qPCR)

Quantitative PCR (qPCR or real-time quantitative PCR—RT-qPCR) is a method to quantify selected polynucleotide sequence by amplifying its initial concentration to a detectable level.

Real-time quantitative PCR induces amplification of nucleic acid amount in sample. This amplification is strongly non-linear and for simplification considered as exponential in its most progressive phase. The amplification of selected locus of DNA is achieved by repeated cycles of set temperature program that facilitates DNA replication by polymerase enzyme. The locus to be amplified is delimited by pair of primers that anneal to the template by molecular affinity and facilitate polymerization of new strand of DNA from supplied essential nucleotides by polymerase enzyme. Eventually, the finalized double stranded DNA product melts into two single stranded molecules by elevated temperature. To clearly separate and facilitates each of these fundamental steps, temperature regime is controlled and repeated in every cycle by the PCR thermal cycler.

The fundamental improvement from a qualitative towards quantitative method was facilitated by addition of fluorescence emitting agent into reaction mix whose fluorescence emission can be monitored throughout the reaction progress. Added into reaction, fluorescence emitting agent closely reflects the current concentration of the nucleic acid mass formed cycle by cycle by interacting with it. The signal emitted by interaction of signaling agent with reaction product is monitored once per cycle and when strengthened enough, the cycle number or its fraction is recorded at this threshold. In this way the so-called threshold cycle (Ct) is obtained. To say when the threshold signal of an individual sample has been reached, qualified arbitrary decision is made or a computing procedure is employed.

Detection of Bacterial Species in Clinical Samples Using Species Specific Quantitative PCR (qPCR) Assays

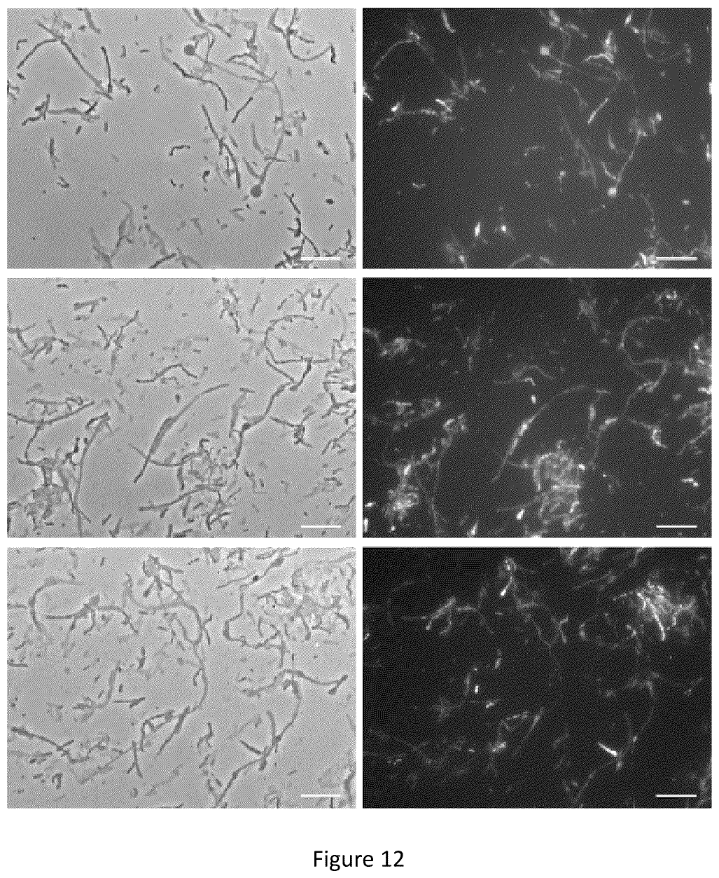

Quantitative PCR (qPCR) assays were designed and validated to detect bacterial species including the four novel species, Fenollaria sp. nov. (FIG. 12), Varibaculum sp. nov., Peptoniphilus sp. nov., and Porphyromonas sp. nov., plus other target bacteria (Table 7). Primers were designed to unique regions (Table 8), and assays were carried out using a LightCycler® 480 instrument (Roche Diagnostics Ltd) with SYBR Green 1 Master and real-time detection of PCR products. Species specific standards were included for comparison and quantification for each assay in 384 well format (8 μl reaction volume per well). PCR amplicons were checked by melt curve analyses, gel electrophoresis and Sanger sequencing.

### Bacteria Genome Sequencing and Assembly

Bacterial DNA was extracted from isolated colonies using Qiagen QIAamp columns as described above and prepared for sequencing with Nextera XT library preparation according to manufacturer's instructions. Sequencing was completed on an Illumina MiSEQ instrument using V3 reagents, 600 cycle flow cell with read lengths up to 2×300 bp. MinION nanopore sequencing was carried out as previously described [53] with the following protocol changes, DNA was extracted with Qiagen G-tip protocol to reduce DNA shearing and the library preparation was completed with Oxford Nanopore Technologies Rapid 1 D sequencing library prep kit and sequenced using MinION SpotON Flow cells (R9.4) as per manufacturer's instructions. Sequence read quality was evaluated using FastQC v0.11.5 (https://www.bioinformatics.babraham.ac.uk/projects/fastqc/). Based on the information from FastQC report, sequence reads were trimmed for low quality and filtered for adapter contamination using Trimmomatic v0.36 [54] and reads less than 35 nucleotides were discarded. Resulting paired and single end reads were used for genome assembly using SPAdes v3.9.0 [55] using multi k-mer approach with parameters ‘-k 21,33,55,77,99,127-careful’. Resulting assembly is filtered by removing contigs less than 500 bp and contigs with mean coverage less than ⅓rd of mean genome coverage. Resulting assembly is evaluated for completeness by BUSCO [56] analysis pipeline. Genomes with more than 90% completeness based on BUSCO analysis were used for downstream analysis by performing de novo annotation with Prokka v1.11 with arguments ‘-kingdom Bacteria—mincontiglen 300-addgenes [57].

### Metabolic Pathway Searching

Predicted protein sequences from Prokka annotation were searched against InterPro protein signature databases using InterProScan REST api v5.29-68.0 [58]. Analysis was primarily focussed on retrieving GO, KEGG, Reactome and Metacyc pathway information for each protein.

### Phylogenetic Analysis

Protein sequences of 16 ribosomal proteins (S3, S8, S10, S17, S19, L2, L3, L4, L5, L6, L14, L15, L16, L18, L22 and L24) were downloaded from NCBI (https://www.ncbi.nlm.nih.gov/) for 1500 selected bacterial genomes. Selected 16 ribosomal protein sequences from assembled bacterial isolates (n=46) from the current study were combined. A concatenated ribosomal protein alignment was constructed by individually aligning each of the ribosomal protein using MAFFT v7.309 [59]. Each ribosomal protein multiple sequence alignments were trimmed to remove poorly aligned regions using trimAl v1.4.rev15 [60]. Resulting 16 trimmed alignments were concatenated to form a final alignment comprising of 383 bacterial genomes and 2382 amino-acid positions. Concatenated proteins alignments were used to generate a maximum likelihood tree with RAxML v8.2.9 [61] with PROTGAMMALG as model parameter, and with 100 bootstrap replicates conducted with the rapid bootstrapping algorithm (raxmlHPC-PTHREADS-AVX-T 16-f a-x 88-m PROTGAMMALG-p 117-N 100). Phylogenetic tree and clade diagrams were generated using ggtree v1.12.0 in R v3.5.0.

The 16S sequences of the novel isolates were checked by running updated BLAST searches (last accessed September 2020), we noted that our isolated Fenollaria sporofastidiosus sp. nov. isolates demonstrated a high similarity to entry Accession: CP027242.1 ‘Peptostreptococcaceae bacterium oral taxon 929 strain W2294’. OrthoANI [62] was used to calculate the average nucleotide identity between the whole genome sequence of our isolates and closely related species. Strain W2294 was only 85.04% and 85.35% similar to our Fenollaria sporofastidiosus sp. nov. isolates. Even though the 16S sequence was highly similar, the whole genome sequence data and other analyses indicate that Fenollaria sporofastidiosus sp. nov. is a novel species.

### Searching for Pathogens in ICGC Datasets

Unmapped reads from human aligned BAM files of 227 (n=204 with clinical follow up, Table 14) ICGC patient blood and prostate tissue sequencing data were classified. A curated Burrow-Wheeler Aligner (BWA) alignment database was setup using 75 genomes that comprised isolates cultured and assembled in the current study, in addition to strains frequently identified by a Kraken analysis along with human genome reference GRCh38 (Table 15—list of genomes used). Reads were selected for downstream analysis, from BWA alignments, with a mapping quality of 20 (99.9% probability that read comes from aligned genome). Alignments>=50 bp are used for downstream analysis and allowed one gap. Sequences from these aligned regions of the genome were extracted and low complexity regions filtered using dustmasker from BLAST+v2.6.0. Sequences with >50 bp of usable sequence after complexity masking were blasted against NCBI-nt database (dated 1 Apr. 2017). Selected regions matching human and related primates from Hominidae were discarded. Selected regions matching respective genera or uncultured sequence or missing taxa name were used for downstream analysis. Assemblies with >200 bp of their genome covered from this analysis were marked as present in the sample.

### RNA Sequencing Metatranscriptomics

SEPATH [63] using Kraken was used to analyse RNA sequencing data (trimming parameters: minimum base quality=25, sliding window=4, minimum read length=35). Human read depletion was carried out using a database consisting of GRCh38 human reference including decoys. All other parameters were as standard. The Kraken database used was a custom-built database containing all genomes on NCBI RefSeq above scaffold level assembly. Kraken reports were analysed with R (v3.5.3), classifications<100 reads assigned to any given clade were set to zero to limit false positive classification. To help address the issue of contamination, any genera suggested as a potential contaminant by Salter et al. 2014 [70] was removed. The community matrix was transformed to presence/absence by using the decostand function (‘vegan’ R package).

### Survival Analysis

Survminer (R package) was used to produce survival curves using default parameters (log-rank test). Taxa were declared as positive for RNA seq and ICGC approach as outlined in previous sections. A minimum relative abundance of 5% was applied to 16s taxa. Progression events were determined by the earliest of the following: appearance of prostate cancer associated metastases on imaging, an increase in histological staging, prostate cancer associated death or an increase in plasma PSA sufficient to affect the course of clinical treatment. Incidents that were not considered progression events include: development of a secondary tumour, non-prostate cancer associated death or a radical prostatectomy. Univariate Cox proportional hazards models were fitted with the coxph function (survival package). A random-effect meta-analysis based on log hazard ratios and their 95% confidence intervals was carried out with the metagen function (meta package) for three technologies: 16S, RNAseq, ICGC.

### Bacteria Novel Species New Proposed Names and Accession IDs

The new bacteria species were named according to the code and guidance presented in ref [64]. Fenollaria sp. nov., new proposed name: Fenollaria sporofastidiosus sp. nov. (sporofastidiosus, sporo spore-forming and fastidiosus in honour of Dr Sidney Finegold who proposed this name by e-mail communication, in relation to fastidious growth requirements). Varibaculum sp. nov., new proposed name: Varibaculum prostatecancerukia sp. nov. (prostatecancerukia, in thanks to the charity Prostate Cancer UK). Peptoniphilus sp. nov., new proposed name: Peptoniphilus rachelemmaiella sp. nov., in relation to two authors who contributed to the anaerobic bacteria culture and knowledge. Plus also Porphyromonas sp. nov., new proposed name: Porphyromonas bobii sp. nov. (bobii, in honour of Bob Champion and the Bob Champion Cancer Trust who have contributed so much to research on prostate cancer). Further descriptions of the novel species and other relevant details are presented in Table 3, FIG. 8 and FIG. 15. The novel bacterial isolates assembled genome sequence data have been deposited in the European Nucleotide Archive, with the following details:

TABLE 1
Sequence deposit details of new bacterial strains

| Primary Accession | Secondary Accession | Title | Tax ID | Scientific Name |
|---|---|---|---|---|
| ERS5804568 | SAMEA8117585 | Varibaculum | 2811781 | Varibaculum sp. |
| (SEQ ID NO: 25) | | prostatecancerukia | | EMRHCC_39 |
| ERS5804567 | SAMEA8117584 | Fenollaria | 2811778 | Fenollaria sp. |
| (SEQ ID NO: 26) | | sporofastidiosus | | EMRHCC_24 |
| ERS5804566 | SAMEA8117583 | Peptoniphilus | 2811779 | Peptoniphilus sp. |
| (SEQ ID NOs: 27-67) | | rachelemmaiella | | EMRHCC_23 |
| ERS5804565 | SAMEA8117582 | Porphyromonas | 2811780 | Porphyromonas sp. |
| (SEQ ID NOs: 68-84) | | bobii | | EMRHCC_6C |

New bacteria species have been deposited in the National Collection of Type Cultures, ECACC and Leibniz Institut Deutsche Sammlung von Mikroorganismen und Zellkulturen (DSMZ) with the following details:

TABLE 2
Biological deposit details of new bacterial strains

| | | Provisional | |
|---|---|---|---|
| | | ECACC | DSMZ |
| | | Accession | Accession |
| Title | Scientific Name | Number | Number |
| Varibaculum | Varibaculum sp. | 21092203 | DSM 34057 |
| prostatecancerukia | EMRHCC_39 | | |
| Fenollaria | Fenollaria sp. | 21092201 | DSM 34056 |
| sporofastidiosus | EMRHCC_24 | | |
| Peptoniphilus | Peptoniphilus sp. | 21092202 | DSM 34055 |
| rachelemmaiella | EMRHCC_23 | | |
| Porphyromonas | Porphyromonas sp. | 21092204 | DSM 34063 |
| bobii | EMRHCC_6C | | |

Four biological deposits were made with the European Collection of Cell Cultures (ECACC), Culture Collections, Public Health England, Porton Down, Salisbury, Wiltshire, SP4 0JG, United Kingdom (accession numbers 21092201, 21092202, 21092203 and 21092204) on 22 Sep. 2021. Four biological deposits were made with the Leibniz Institute DSMZ-German Collection of Microorganisms and Cell Cultures, Inhoffenstr. 7B, D-38124, Braunschweig, Germany (accession numbers 34056, 34055, 34063 and 34057) on 23 Sep. 2021.

The sequence assemblies for Varibaculum prostatecancerukia and Fenollaria sporofastidiosus (SEQ ID NOs:25 and 26) contain contiguous sequences assembled from short reads (sometimes with additional long read sequencing). Sometimes it is not possible (and/or not desirable) to assemble a bacterial genome into one single long contiguous sequence. The sequence assemblies for Peptoniphilus rachelemmaiella and Porphyromonas bobii (SEQ ID NOs:27-84) contain multiple contigs ordered from largest to smallest for each bacterium. Any part of the bacterial genome which is susceptible to detection via PCR techniques may be useful for identifying and/or detecting the presence of bacteria in a biological sample.

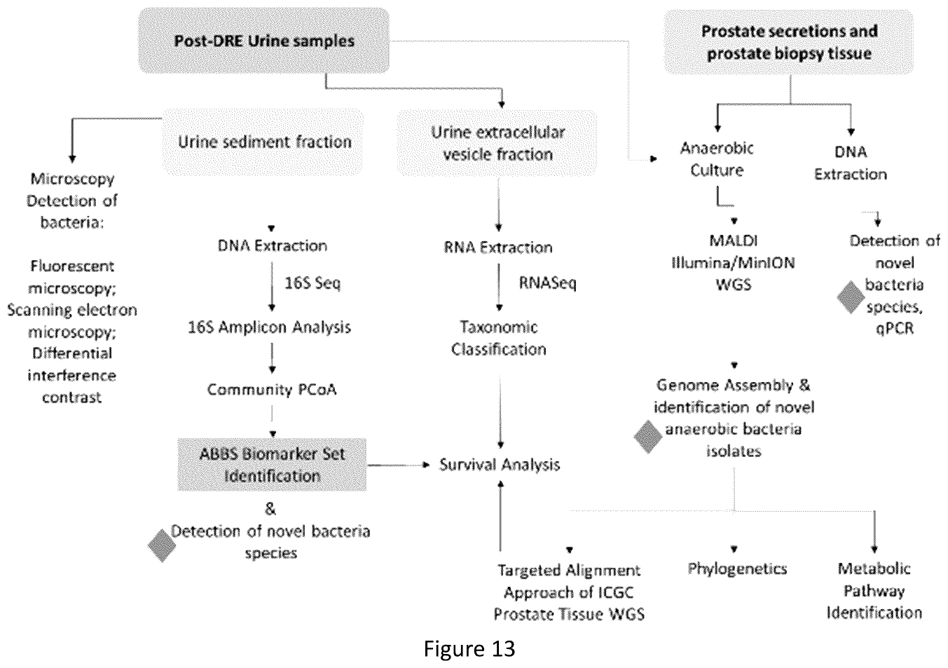

An overview flow chart of the analyses in this study highlighting the identification of ABBS and novel species are shown in FIG. 13.

Steps taken for minimising and assessing contamination according to RIDE criteria (Report, Include, Determine, Explore criteria checklist).

### Sample Collection and Processing

The experimental design was established to reduce the potential for microbial contamination. Tissue biopsies and prostate fluid secretions were collected from excised prostates post-prostatectomy whilst still in the operating theatre using sterile equipment. Where possible all samples were processed within a clean microbiological safety cabinet. The picking of pure colonies from cultures was completed in a HEPA filtered anaerobic cabinet with sterile equipment (Don Whitley Scientific, UK).

Steps taken to reduce contamination in the urine sediment 16S sequencing data and qPCR datasets: 16S amplicon sequencing protocol utilised Mycoplasma hyorhinis as a positive control and several negative control samples (no template controls, elution buffer controls and blank bead-beating extraction samples). One negative control sample (V1V3, ID: M93.L) contained 99.96% relative abundance of Mycoplasma, 0.014% Ralstonia, 0.007% Achromobacter, 0.007% Pelomonas, 0.007% Undibacterium. Ralstonia and Undibacterium classifications from V1V3 16S were present in some samples in the rest of the dataset. Ralstonia was in 22 samples (median 0.135%, IQR 0.23%, maximum 3.3%). Undibacterium was present in 19 samples (median 0%, IQR 0.61%, maximum 0.99%). Classifications obtained in negative controls are therefore unlikely to have a significant impact on overall community structure or on genera in the ABBS list. Regarding the qPCR assays, species specific standards were included for quantification of the species-specific qPCR assays in a 384 well format. PCR amplicons were checked by melt curve analyses, gel electrophoresis and additional Sanger sequencing.

Sequence data analysis, RNAseq and whole genome sequence data consideration of contamination: The taxonomy of isolates was investigated by several approaches including MALDI, BLAST searches of 16S sequences, pro-genomes classifier (data not shown), 16S protein phylogenetics and completeness was examined with BUSCO. One isolate appeared mixed (strain 02_15) which was suspected in part to be Fusobacterium nucleatum. This strain was excluded from any additional analysis. RNA sequencing of urine extracellular samples and ICGC WGS samples were not sequenced locally and not necessarily designed for microbial detection, (except sterile molecular biology grade reagents and equipment were used for RNA extraction). For these cohorts, no negative control samples were sequenced and so we are unable to comment in this regard. The sample-genus community matrix from RNA sequencing was filtered for a minimum of 100 classified reads to help limit the prevalence of contamination/false positive results. Additionally, all common contaminant genera in negative controls suggested by Salter et al. 2014 [70] were removed from analysis. This is with the caveat that this list also includes some biologically relevant bacterial genera such as Escherichia. Opposingly, some contaminant genera likely remain in the community matrix such as Agrobacterium which is a suspected contaminant (or a false positive result) because it is frequently identified in soil and is a known plant pathogen [65] with limited documentation as a widespread human commensal/pathogen. The extraction and application of ABBS genera helps to remove the potential for taxa such as Agrobacterium to impact results.

The ICGC prostate tissue WGS data targeted alignment approach was developed with contamination in mind. As well as including in-house isolates isolated from the prostate, we also included reference genomes including some prevalent contaminants as indicated by Salter et al. 2014 [70] that we frequently noted including: Stenotrophomonas, Ralstonia, Escherichia and Toxoplasma to name a few. Additionally, sequences were dustmasked to remove low complexity and regions matching human and related primates from homindae were removed to help limit false positive classifications from human sequences.

Many of the ABBS genera (Fenollaria, Ezakiella, Peptoniphilus, Porphyromonas, Anaerococcus, and Fusobacterium), originally defined based on the 16S urine community analysis (higher median relative abundance>2% in cluster 1 compared to the rest of the dataset and p<0.1, Table 9) contain well documented human pathogens. Furthermore, other studies have documented the presence of Peptoniphilus, Porphyromonas, Anaerococcus, and Fusobacterium in urine (reviewed in [66]) which might diminish the likelihood that any of them are contaminant genera. A recent meta-analysis suggests that Fusobacterium, Anaerococcus and Porphyromonas can occur in negative control samples [67]. However, these three genera are also documented human pathogens/commensals [68,69]. Of the ABBS positive samples with prostate cancer in the RNA sequencing dataset: 14% were positive for Ezakiella, 64% positive for Peptoniphilus, 36% positive for Porphyromonas, 57% positive for Anaerococcus, 57% positive for Fusobacterium. Therefore Fusobacterium, Anaerococcus and Porphyromonas have a high prevalence in ABBS positive samples. We have obtained pure cultures of many of the ABBS genera which indicates that they are present and viable in clinical samples and thus not contamination or hits from sequences in the ‘kitome’. It is reassuring that the trend with survival as shown in this study is replicated by distinct approaches that are considerate of contamination.

### Biopsies

A prostate biopsy involves taking a sample of the prostate tissue, for example by using thin needles to take small samples of tissue from the prostate. The tissue is then examined under a microscope to check for cancer.

There are two main types of prostate biopsy—a TRUS (trans-rectal ultrasound) guided or transrectal biopsy, and a template (transperineal) biopsy. TRUS biopsy involves insertion of an ultrasound probe into the rectum and scanning the prostate in order to guide where to extract the cells from. Normally 10 to 12 small pieces of tissue are taken from different areas of the prostate.

A template biopsy involves inserting the biopsy needle into the prostate through the skin between the testicles and the rectum (the perineum). The needle is inserted through a grid (template). A template biopsy takes more tissue samples from more areas of the prostate than a TRUS biopsy. The number of samples taken will vary but can be around 20 to 50 from different areas of the prostate.

### Prostate Cancer Treatment

Patients with metastatic disease are primarily treated with hormone deprivation therapy. However, the cancer invariably becomes resistant to treatment leading to disease progression and eventually death. Treatment of patients with metastatic prostate cancer is clinically very challenging for a number of reasons, which include: i) the variability in patient response to hormone treatment (i.e. time prior to relapse and becoming castrate resistant), ii) the detrimental effects of hormone manipulation therapy on patients and iii) the myriad new treatment options available for castrate resistant patients. In some cases, treatment of prostate cancer can be placing the patient under active surveillance.

The response to hormone manipulation/ablation therapy is highly variable. Some men fail to respond to treatment while others relapse early (i.e. within 6 months), the majority relapse within 18 months (late relapse) and the rest respond well to the treatment often taking several years before relapsing (delayed relapse). Early identification of patients who will have a poor response will provide a clinical opportunity to offer them a different treatment approach that may perhaps improve their prognosis. However, there is no means currently to identify such patients except for when they exhibit biochemical progression with rising PSA level (e.g. serum PSA level), or become clinically symptomatic, in which case they get offered a different treatment strategy. This regime however goes hand in hand with a number of detrimental effects such as bone loss, increased obesity, decreased insulin sensitivity increasing the incidence of diabetes, adversely altered lipid profiles leading to cardiovascular disease and an increased rate of heart attacks. For these reasons offering hormone manipulation requires a lot of clinical consideration particularly as most of the patients requiring such treatment are elderly patients and such treatment could overall be detrimental rather than beneficial.

Due to ever-emerging new treatments or second line therapies for patients with advanced metastatic cancer in the past decade, the treatment of men with castrate resistant prostate cancer is dramatically changing. Prior to 2004, the only treatment option for these patients was medical or surgical castration then palliation. Since then several chemotherapy treatments have emerged starting with docetaxel, which has shown to improve survival for some patients. This was followed by five additional agents (FDA-approved) including new hormonal agents targeting the androgen receptor (AR) such as the AR antagonist Enzalutamide, agents to inhibit androgen biosynthesis such as Abiraterone, two agents designed specifically to affect the androgen axis, sipuleucel-T, which stimulates the immune system, cabazitaxel chemotherapeutic agent and radium-223, a radionuclide therapy. Other treatments include targeted therapies such as the PI3K inhibitor BKM120 and an Akt inhibitor AZD5363. Therefore, it is crucially important to be able to identify patients that would benefit from these treatments and those that will not. Identification of prognostic indicators capable of predicting response to hormone manipulation and to the above list of alternative treatments is very important and would have great clinical impact in managing these patients. In addition, the only current clinically available means to diagnose metastasis is by imaging. Markers that are being put forward include circulating tumour cells and urine bone degradation markers. A test for metastasis per se could radically alter patient treatment.

Prostate cancers can be staged according to how advanced they are. This is based on the TMN scoring as well as any other factors, such as the Gleason score and/or the PSA test. The staging can be defined as follows:

### Stage I:

T1, N0, M0, Gleason score 6 or less, PSA less than 10
OR
T2a, N0, M0, Gleason score 6 or less, PSA less than 10

### Stage IIA:

T1, N0, M0, Gleason score of 7, PSA less than 20
OR
T1, N0, M0, Gleason score of 6 or less, PSA at least 10 but less than 20:
OR
T2a or T2b, N0, M0, Gleason score of 7 or less, PSA less than 20

### Stage IIB:

T2c, N0, M0, any Gleason score, any PSA
OR
T1 or T2, N0, M0, any Gleason score, PSA of 20 or more:
OR
T1 or T2, N0, M0, Gleason score of 8 or higher, any PSA

### Stage III:

T3, N0, M0, any Gleason score, any PSA

### Stage IV:

T4, N0, M0, any Gleason score, any PSA
OR
Any T, N1, M0, any Gleason score, any PSA:
OR
Any T, any N, M1, any Gleason score, any PSA

In the present invention, an aggressive cancer is defined functionally or clinically: namely a cancer that can progress. This can be measured by PSA failure. When a patient has surgery or radiation therapy, the prostate cells are killed or removed. Since PSA is only made by prostate cells the PSA level in the patient's blood reduces to a very low or undetectable amount. If the cancer starts to recur, the PSA level increases and becomes detectable again. This is referred to as “PSA failure”. An alternative measure is the presence of metastases or death as endpoints.

Prostate cancer can be scored using the Prostate Imaging Reporting and Data System (PI-RADS) grading system designed to standardise non-invasive MRI and related image acquisition and reporting, potentially useful in the initial assessment of the risk of clinically significant prostate cancer. A PI-RADS score is given according to each variable parameter. The scale is based on a score “Yes” or “No” for Dynamic Contrast-Enhanced (DCE) parameter, and from 1 to 5 for T2-weighted (T2W) and Diffusion-weighted imaging (DWI). The score is given for each lesion, with 1 being most probably benign and 5 being highly suspicious of malignancy:
PI-RADS 1: very low (clinically significant cancer is highly unlikely to be present)
PI-RADS 2: low (clinically significant cancer is unlikely to be present)
PI-RADS 3: intermediate (the presence of clinically significant cancer is equivocal)
PI-RADS 4: high (clinically significant cancer is likely to be present)
PI-RADS 5: very high (clinically significant cancer is highly likely to be present)

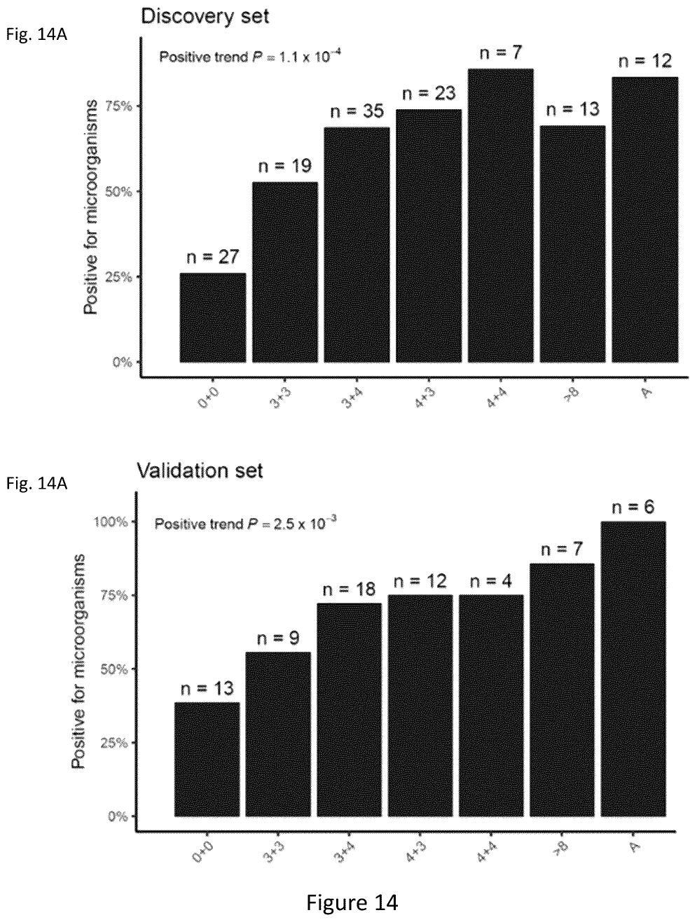

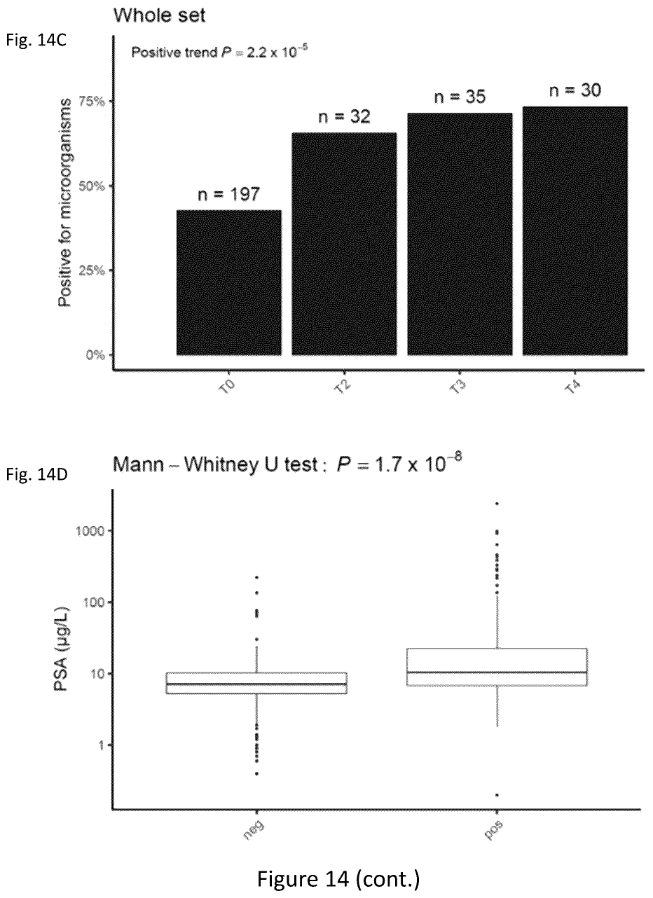

Increase in Gleason score, stage as defined above or PI-RADS grade can also be considered as progression. However, the presence or absence of ABBS bacteria is independent of Gleason, stage and PI-RADS. It provides additional information about the development of aggressive cancer in addition to Gleason, stage and PI-RADS. It is therefore a useful independent predictor of outcome. Nevertheless, the presence or absence of ABBS bacteria can be combined with Gleason, tumour stage and/or PI-RADS score. FIG. 14 shows data demonstrating the association of presence of bacteria in urine sediment with prostate cancer clinical grade, stage and PSA.

In some methods of the invention the identification of ABBS bacteria can be used alongside MRI to aid decision making on whether to biopsy or not, particularly in men with PI-RADS 3 and 4. The presence of ABBS bacteria could also be used to confirm the absence of clinically significant prostate cancer in men with PI-RADS 1 and 2.

By “clinical outcome” it is meant that for each patient whether the cancer has progressed. For example, as part of an initial assessment, those patients may have prostate specific antigen (PSA) levels monitored. When it rises above a specific level, this is indicative of relapse and hence disease progression. Histopathological diagnosis may also be used. Spread to lymph nodes, and metastasis can also be used, as well as death of the patient from the cancer (or simply death of the patient in general) to define the clinical endpoint. Gleason scoring, cancer staging and multiple biopsies (such as those obtained using a coring method involving hollow needles to obtain samples) can be used. Clinical outcomes may also be assessed after treatment for prostate cancer. This is what happens to the patient in the long term. Usually the patient will be treated radically (prostatectomy, radiotherapy) to effectively remove or kill the prostate. The presence of a relapse or a subsequent rise in PSA level (e.g. serum PSA level) (known as PSAfailure) is indicative of progressed cancer.

Accordingly, any of the methods of the invention may be carried out in patients in whom prostate cancer is suspected. Importantly, the present invention allows a prediction of cancer progression before treatment of cancer is provided. This is particularly important for prostate cancer, since many patients will undergo unnecessary treatment for prostate cancer when the cancer would not have progressed even without treatment.

Proteins can also be used to determine expression status, and suitable methods to determine expressed protein levels are known to the skilled person.

The present invention shall now be further described with reference to the following examples, which are present for the purposes of illustration only and are not to be construed as being limiting on the invention.

### EXAMPLES

### Example 1—Bacteria in Urine are Associated with Disease Progression Risk in Prostate Cancer

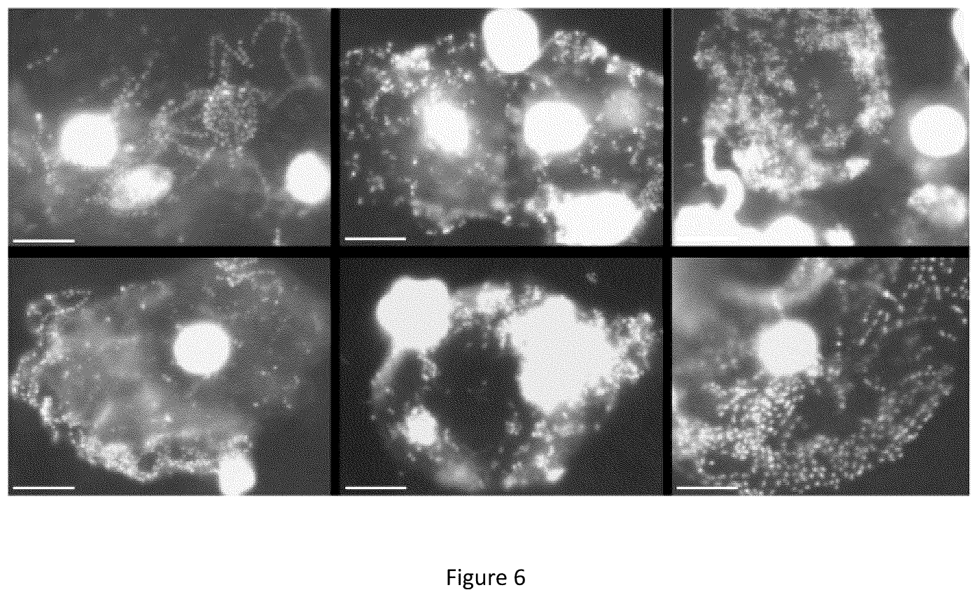

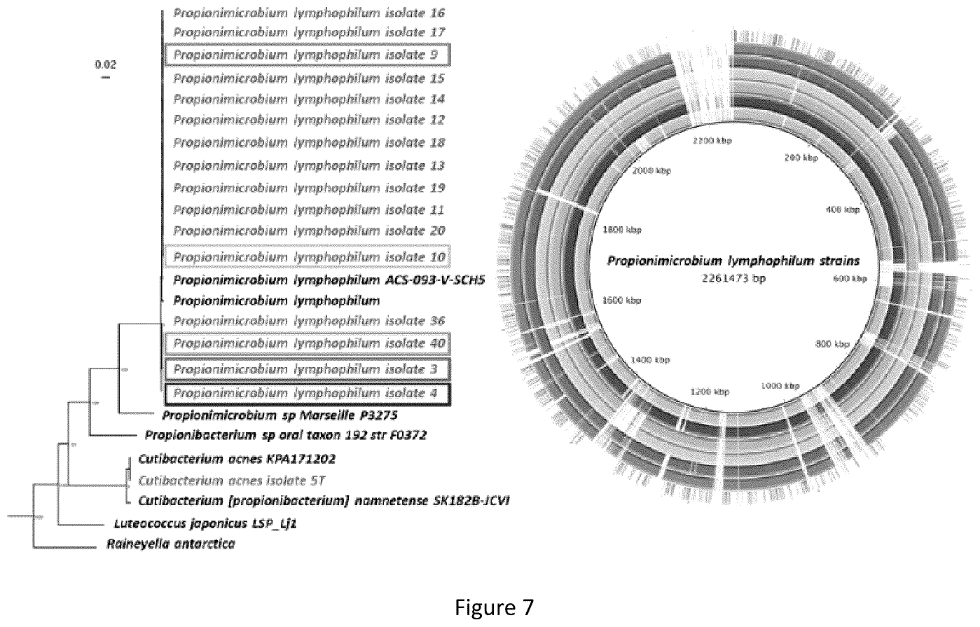

Examination of post-Digital Rectal Examination (DRE) urine sediments revealed background DNA staining suggestive of bacteria in ˜50% samples, which was also supported by scanning electron microscopy (FIGS. 1 A and B, FIG. 5). To test the importance of this observation we analysed urine samples from men undergoing assessment for prostate cancer (n=300) or from a haematuria clinic (n=18). Background DNA staining corresponding to bacteria was more common from men with D'Amico high risk or advanced prostate cancer (FIG. 1C) with a significant positive trend linking the presence of bacteria to a higher risk of disease progression. In the discovery set of 215 samples a chi-squared test for trend in proportions gave a strong association (P=2.2×10⁻⁶) which was confirmed in the validation set of 103 (P=4.5×10⁻⁵), and bacteria were also observed in much larger aggregates (FIGS. 5 A and B). For the combined data set the proportion of patients with these aggregates in their urine (FIG. 5) was also significantly linked (P=0.006; FIG. 5C) to prostate cancer risk of progression. The bacteria also appeared in some cases to be intracellular within human cells (FIG. 6). We concluded that the presence of bacteria in urine cell sediment was correlated with cancer having higher risk of disease progression and may represent a useful prognostic indicator.

### Example 2—Culture Confirms New Species of Bacteria from the Urine of Prostate Cancer Patients

To identify the bacteria involved, we initially used a standard 16S sequencing approach using accepted protocols and controls to avoid contamination [70,71]. The bacterial community structure, identified using 16S rRNA amplicon pyrosequencing on urine sediments from 46 men (24 with a diagnosis of prostate cancer), revealed 1614 bacterial operational taxonomic units (OTUs). There was no clear association between the number of OTUs detected and prostate cancer progression risk: an average of 168 OTUs (range: 64-265) in samples from non-cancer patients; 130 OTUs (range: 67-237) in samples from D'Amico low/intermediate risk prostate cancer and 171 OTUs (range: 81-290) in samples from high risk and advanced. During these analyses however, we observed many OTUs that lacked assignment at deeper than genus level and some that were assigned only at family level. To explore this result, we performed direct searches of unassigned OTU sequences against the NCBI dataset but again found no exact matches. We concluded that the post-DRE urine contained novel bacterial species that have not yet been cultured and characterised and so we attempted to culture them.

We applied a fastidious anaerobic culture protocol for culturing bacteria previously considered to be “unculturable” [72] to post-DRE urine sediments and also to prostate cancer secretions and biopsies obtained after prostatectomy. Strict anaerobic culture protocols yielded 39 bacterial isolates from post-DRE urine (FIG. 15) and 8 isolated from prostate cancer secretions. Whole genome sequencing (Illumina sequencing for all n=47 isolates and Oxford Nanopore Technologies hybrid assemblies for candidate novel species) resulted in high quality genomes contigs (1 to 515 per bacteria isolated). Most of the anaerobic bacterial isolates from post-DRE urine sediments were from the phyla Firmicutes, class Clostridia, including genera Peptoniphilus, Fenollaria and Anaerococcus (FIG. 15). Sixteen isolates of Propionimicrobium lymphophilium from three different urine samples demonstrated considerable genetic variation (FIG. 8). Prostate secretions yielded bacteria from the genera Porphyromonas, Staphylococcus, Streptococcus and Cutibacterium (FIG. 15).

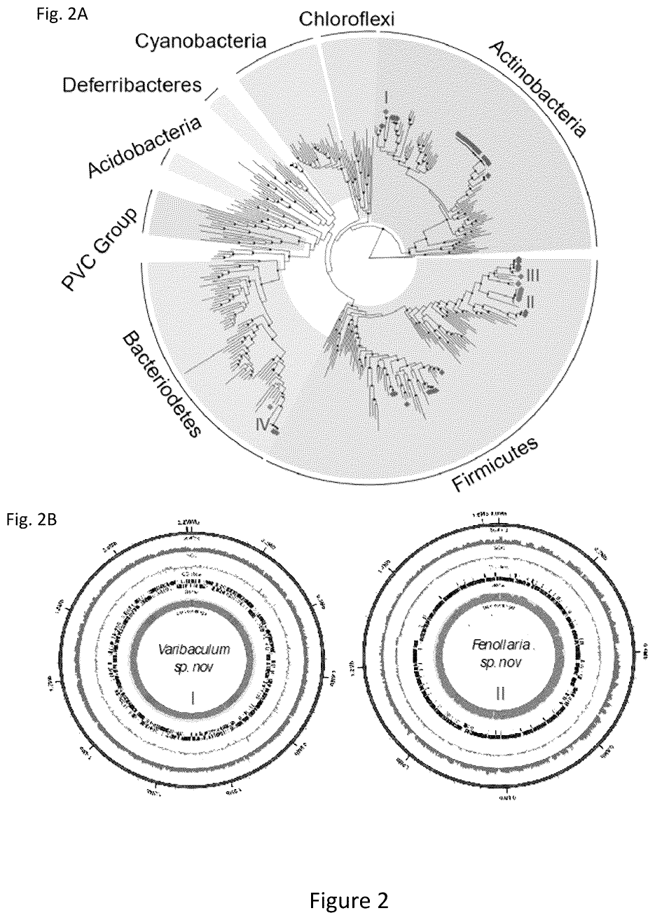

Higher-resolution phylogenetic analysis was obtained by aligning selected full-length ribosomal protein gene sequences from the unclassified isolates to the same genes from known bacterial species [41], (FIG. 2a). This allowed us to identify four novel species (Table 3). The novel species, defined as sequence similarity less than 97% to the closest published assemblies [73], were from the phyla Firmicutes, (Fenollaria sp. nov. and Peptoniphilus sp.nov.), Actinobacteria (Varibaculum sp. nov.) and Bacteroidetes (Porphyromonas sp. nov.). Further details on novel species and isolates are in FIG. 8 A-D and Table 6.

TABLE 3
Novel bacteria species isolated. Novel species isolated from clinical samples, including
taxonomy and proposed new species name

| Novel Species | Novel species | Novel species | Novel species | Reference Strain |
|---|---|---|---|---|
| Isolated Bacteria | belonging to | belonging to | belonging to | ID new proposed |
| ID | Phyla | Class | Genus | name |
| Fenollaria sp. nov. | Firmicutes | Clostridia | Fenollaria | Fenollaria |
| | | | | sporofastidiosus sp. |
| | | | | nov. |
| Peptoniphilus sp. | Firmicutes | Clostridia | Peptoniphilus | Peptoniphilus |
| nov. | | | | rachelemmaiella sp. |
| | | | | nov. |
| Varibaculum sp. | Actinobacteria | Actinobacteria | Varibaculum | Varibaculum |
| nov. | | | | prostatecancerukia |
| | | | | sp. nov. |
| Porphyromonas | Bacteroidetes | Bacteroidia | Porphyromonas | Porphyromonas |
| sp. nov. | | | | bobii sp. nov. |

We confirmed the presence of all four novel species in urine cell sediment samples with in-house designed specific qPCR assays (6 to 65% of samples, Tables 7 and 8). Two of the novel species (Peptoniphilus sp. nov., Varibaculum sp. nov.) were also detected by qPCR in prostate tissue (2.8 to 8.6%) and all four novel species in prostate secretions (2.8 to 17%) consistent with a prostate-urine reflux loop where there is an exchange of bacteria between the urine and prostate.

### Example 3—Taxonomic Resolution Allows the Identification of Bacterial Genera Potentially Associated with Disease Progression

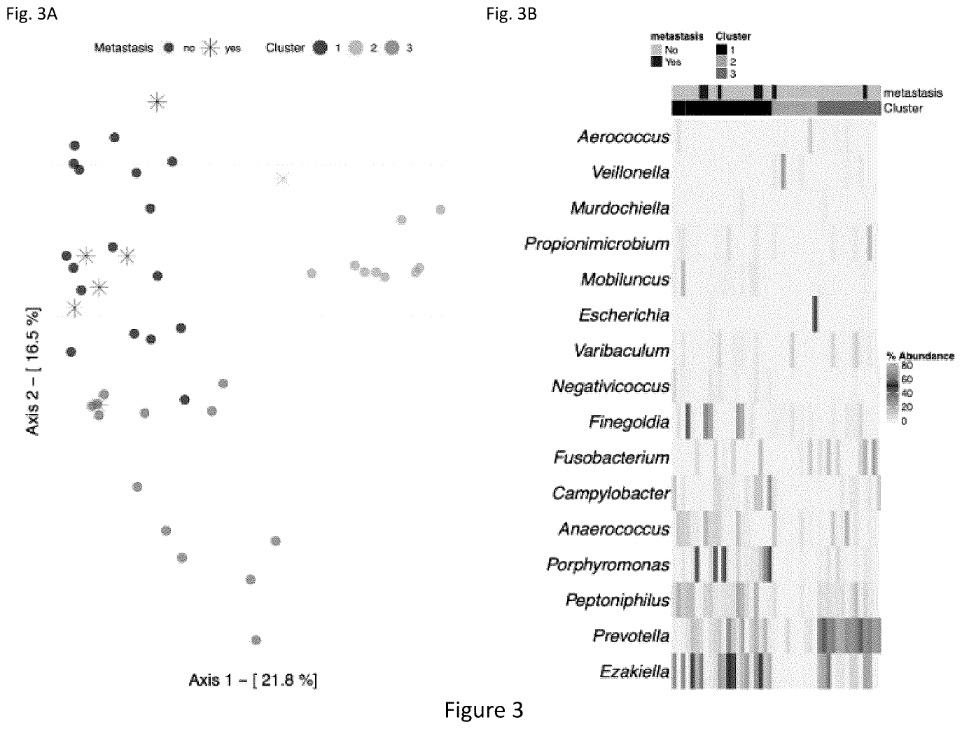

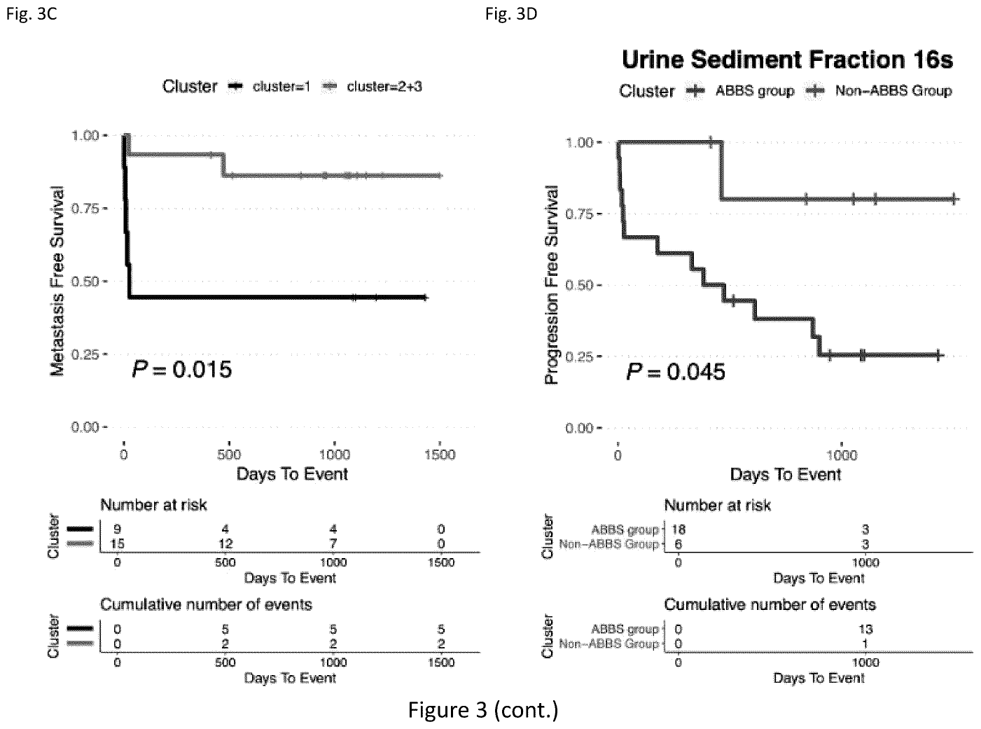

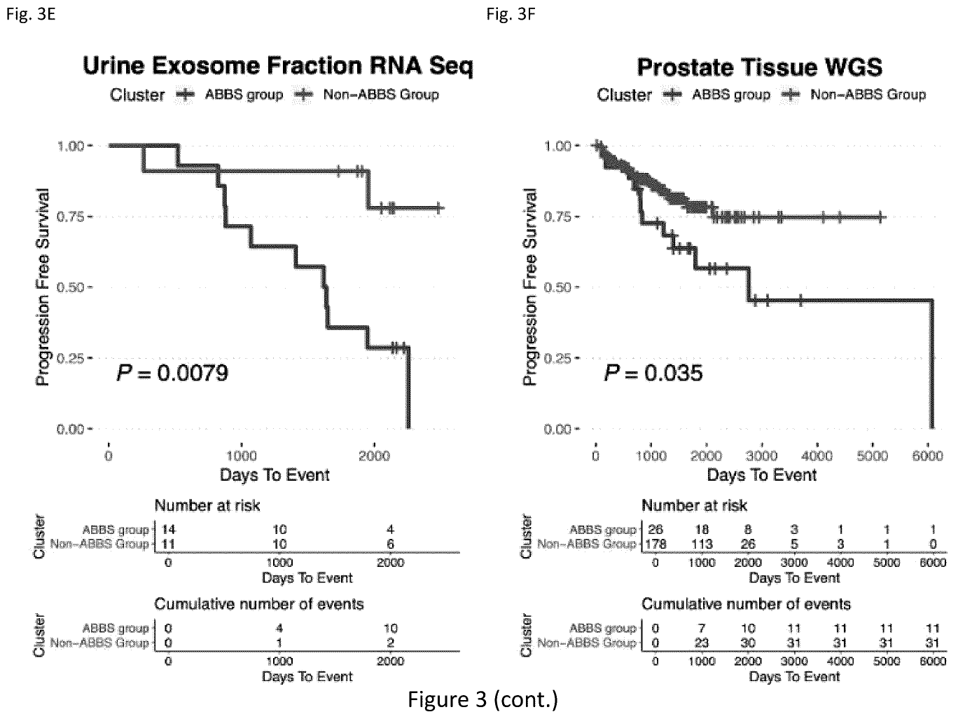

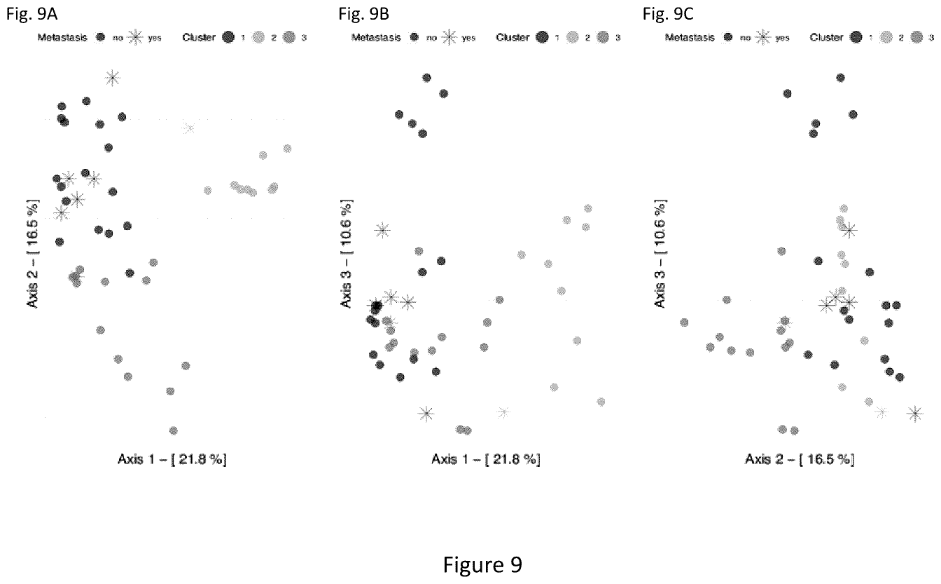

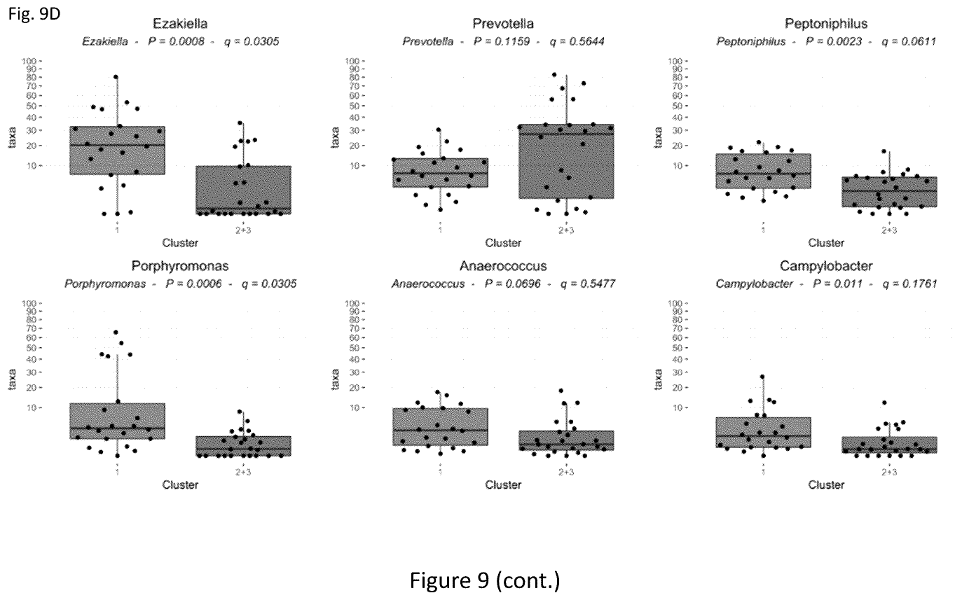

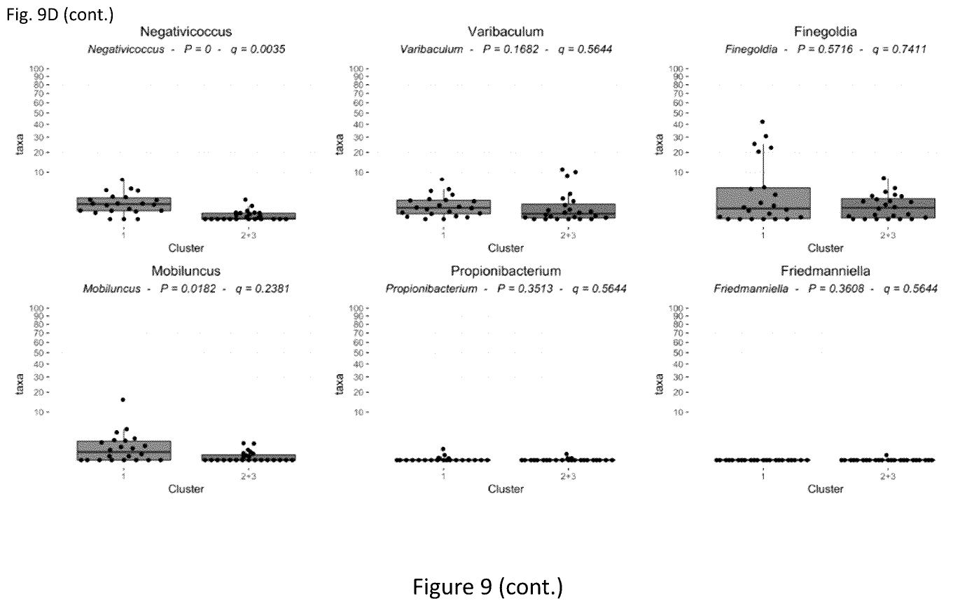

Having identified the putative novel species, we linked them to the identified 16S OTUs. Clustering on the relative abundance of the 16S OTUs, using Principal Coordinate Analysis (PCoA), allowed us to associate bacterial communities with disease state (FIG. 3). With OTU data from 46 men three main clusters (using Manhattan distance and k-means clustering) were observed (FIGS. 3 A and B and FIG. 9) explaining 48.9% of variance within the first three principal coordinates (FIG. 3A and FIG. 9 A-C). Patients demonstrating elevated rates of metastases were overrepresented in Cluster 1 (FIG. 3A, stars) compared to the other two clusters (P=0.015; log-rank test. FIG. 3C).

To identify bacterial genera associated with cluster 1 (metastatic group) that could potentially be used as prostate cancer biomarkers, we investigated differences in relative abundance between cluster 1 and the remainder of the dataset (FIG. 9D, Table 9). The genera selected for further study, based on statistical significance and fold difference, were the strict anaerobes Fenollaria/Ezakiella, Peptoniphilus, Porphyromonas and Anaerococcus. Fusobacterium, another anaerobe detected in the 16S data from microscopy positive samples, was also included due to rapidly growing evidence of its association with the development of a range of cancers [74,75]. Co-occurrence plots of the bacteria genera identified from the urine sediment 16S data demonstrated that several of these strict anaerobes are commonly found together in high risk and advanced/metastatic disease (FIG. 11). The five selected bacteria genera (Table 4, Table 10) are referred to as the ABBS (Anaerobic Bacteria Biomarker Set).

TABLE 4
Anaerobic Bacteria Biomarker Set (ABBS). Bacteria associated with a poor prognosis.
ABBS bacteria taxonomy, novel species isolated in this study ( [symbol] )

| Anaerobic | | | | |
|---|---|---|---|---|
| Bacteria | | | | Novel Species and known |
| Biomarker Set: | ABBS | ABBS | ABBS | species isolated by |
| ABBS bacteria | belonging to | belonging to | belonging to | anaerobic culture belonging |
| genera | Phylum | Class | Order | to ABBS genera |
| Fenollaria | Firmicutes | Clostridia | Clostridiales | Fenollaria sp. nov. [symbol] |
| | | | | (Fenollaria sporofastidiosus) |
| Peptoniphilus | Firmicutes | Clostridia | Clostridiales | Peptoniphilus sp. nov. [symbol] ; |
| | | | | (Peptoniphilus |
| | | | | rachelemmaiella) |
| | | | | Peptoniphilus harei |
| Anaerococcus | Firmicutes | Clostridia | Clostridiales | Anaerococcus prevotii |
| Porphyromonas | Bacteroidetes | Bacteroidia | Bacteroidales | Porphyromonas sp. nov. [symbol] ; |
| | | | | (Porphyromonas bobii), |
| | | | | Porphyromonas |
| | | | | asaccharolytica |
| Fusobacterium | Fusobacteria | Fusobacteriia | Fusobacteriales | Fusobacterium nucleatum |

### Example 4—Use of the Anaerobic Bacteria Biomarker Set from the Prostate-Urine Reflux Loop as a Prognostic Biomarker

Processing of urine samples generates the sedimentary faction that was used to detect bacterial DNA fluorescence staining and generate OTU data as described above, and a supernatant fraction that contains prostate derived exosomes. To establish a relationship between the ABBS and cancers that progress clinically we examined both of these fractions. First, using the 16S OTU data (n=24) from urine sediments. Second, using RNA sequencing data from urine exosome supernatant fractions (n=24). Both technologies demonstrated more rapid clinical progression for men when at least one of the ABBS bacteria genera were detected (FIGS. 3 D and E). These two sample sets had six urine samples in common so were not entirely independent.

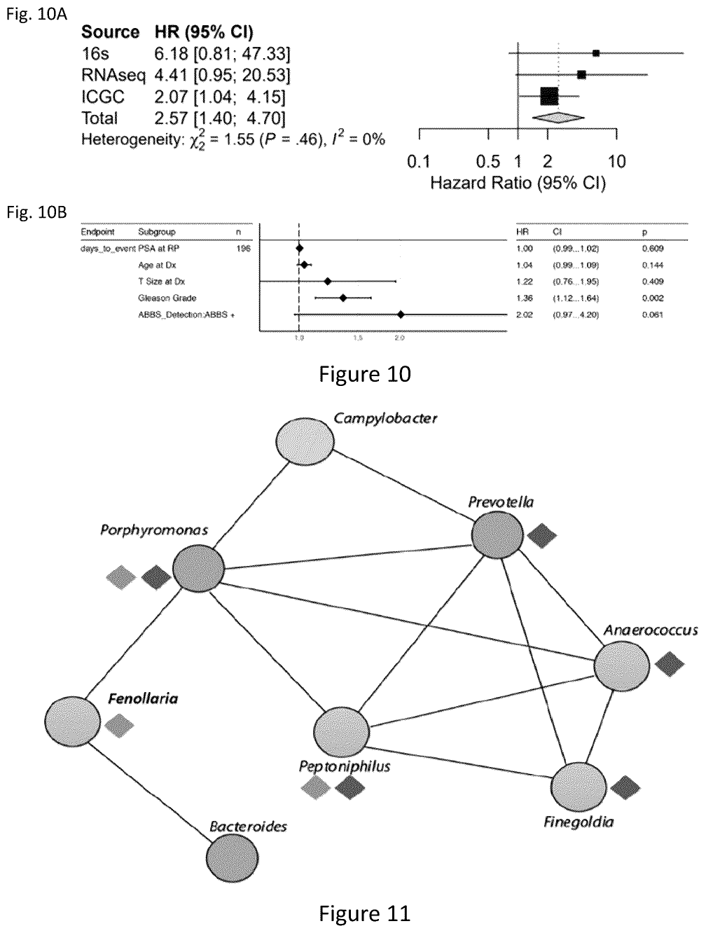

To validate these indicative results, we examined a much larger and entirely independent prostate cancer tissue dataset from the International Cancer Genome Consortium (ICGC) for association of bacterial genera with prostate cancer progression. Although this is human cancer genome data, bacterial DNA is concomitantly sequenced if present in the DNA sample extracted from the prostate cancer tissue. Interrogating data for 204 samples (FIG. 3F) indicated a significantly high rate of clinical progression in donors with at least one of the anaerobic bacterial genera detected, with a hazard ratio of 2.07 (95% Cl: 1.04-4.15). Combining the three sets of data in a meta-analysis gave a hazard ratio of 2.93 (95% Cl: 1.43-6; Cox proportional hazards regression; FIG. 10) for disease progression. These results led us to believe that detecting the presence of anaerobic bacteria that comprise the ABBS in the urinary tract may constitute a prognostic test for the risk of clinical progression in prostate cancer patients. The possibility that these anaerobic bacteria are not only indicative of cancer progression but associated with cancer development and progression was then investigated.

### Example 5—Investigating the Mechanism of Action

We next used whole genome DNA sequence data from bacteria present in the urine and prostate to predict the presence of genes and functional pathways. These studies add to previous studies on prostate cancer that have investigated the association between the presence of microorganisms and inflammation [27,76], and identify a variety of specific molecular mechanisms [39] that are of potential interest for tumour progression and for therapeutic exploitation. Genera within ABBS were predicted to encode components of metabolic pathways that can convert cholesterol to androstenedione (FIG. 4), an immediate precursor for testosterone that is required for prostate cancer growth [77]. This pathway was not present non-ABBS isolates (FIG. 4) The presence of ABBS bacteria could also help explain why prostate cancer patients develop resistance to the drug abiraterone, which completely abates eukaryotic testosterone synthesis through irreversible inhibition of the enzyme CYP17 [77], the eukaryotic enzyme responsible for androstenedione production. Development of drugs that target these bacterial pathways or eradicate the pathogens could remove the bacterial source of androstenedione and improve survival.

Thymidylate synthase (TS) was a common pathway across all bacteria with a skew towards flavin-dependent (FAD) bacterial specific TS activity in ABBS genera (FIG. 4). We note that TS overexpression has been demonstrated as a prognostic marker for aggressive prostate cancer [78] and that TS upregulation in cancer is a resistance marker for the anti-cancer drug 5-fluorouracil (5-FU) [79]. The presence of these bacteria may help explain why advanced prostate cancer has historically demonstrated poor response rates to 5-FU [80]. Fenollaria sp. nov., Peptoniphilus harei and Fusobacterium nucleatum have predicted citrate lyase complex: interestingly reduced citrate is a known predictor of cancer aggression in prostate cancer [81]. Other features for several the ABBS bacteria include the predicted presence of glycine cleavage complex and components of the pathway for biotin synthesis (FIG. 4), these are of interest as potential impact on human host cell metabolism pathways including fatty acid metabolism plus glycine and associated pathways which are known to be perturbed in prostate cancer [25,81,82,83].

TABLE 5
Correlation of bacteria 16S PCR positive samples with
background DNA staining. 16S PCR reactions were carried out
as detailed in the methods section for n = 111 urine samples.
Correlation of the detection of background DAPI staining by
microscopy to the presence of bacterial 16S PCR products
(X²-test, df = 1, P = 9.262e−10) confirmed that the stained
background DNA corresponded to the presence of bacteria.

| | | Positive by microscopy | | |
|---|---|---|---|---|
| | | Yes | No | Total |
| Positive by 16S | Yes | 63 | 13 | 76 |
| Ribosomal PCR | No | 8 | 27 | 35 |
| | Total | 71 | 40 | 111 |

TABLE 6
Novel bacterial species cultured from urine samples and prostate secretions.
Novel species isolated from clinical samples, including the phyla, class, order and genera that the proposed new species
belong to. Details of clinical sample used for culture and isolation of novel anaerobic species, reference strain
IDs and accession numbers.

| Novel Species Isolated | Novel species | Novel species | Novel species belonging | Novel species belonging |
|---|---|---|---|---|
| Bacteria ID | belonging to Phyla* | belonging to Class* | to Order | to Genus |
| Fenollaria sp. nov. [symbol] | Firmicutes | Clostridia | Clostridiales | Fenollaria |
| Peptoniphilus sp. nov. [symbol] | Firmicutes | Clostridia | Clostridiales | Peptoniphilus |
| Varibaculum sp. nov. [symbol] | Actinobacteria | Actinobacteria | Actinomycetales | Varibaculum |
| Porphyromonas sp. | Bacteroidetes | Bacteroidia | Bacteroidales | Porphyromonas |
| nov. [symbol] | | | | |

| | | | Reference | | |
|---|---|---|---|---|---|
| | | Details of clinical | Strain ID | | |
| Novel Species | | sample used for culture | culture | Reference Strain ID | |
| Isolated Bacteria | | and isolation of novel | collection | new proposed | Reference Strain ID |
| ID | Isolates sequenced | species | deposit | name | accession numbers |
| Fenollaria sp. nov. | isolates: 24, 27A, | isolated from urine | Fenollaria sp. | Fenollaria | ERS5804567 |
| [symbol] | 27B, 29, 35 | sample from a patient | nov.: | sporofastidiosus sp. | (SEQ ID NO: 26) |
| | | with high risk prostate | EMRHCC_24 | nov. | and |
| | | cancer | | | SAMEA8117584 |
| Peptoniphilus sp. | isolate 23 | isolated from urine | Peptoniphilus | Peptoniphilus | ERS5804566 |
| nov. [symbol] | | sample from a patient | sp. nov.: | rachelemmaiella | (SEQ ID |
| | | with high risk prostate | EMRHCC_23 | sp. nov. | NOs: 27-67) and |
| | | cancer | | | SAMEA8117583 |
| Varibaculum sp. | isolate 39 | isolated from urine | Varibaculum sp. | Varibaculum | ERS5804568 |
| nov. [symbol] | | sample from a patient | nov.: | prostatecancerukia | (SEQ ID NO: 25) |
| | | who underwent clinical | EMRHCC_39 | sp. nov. | and |
| | | assessment but had no | | | SAMEA8117585 |
| | | evidence of prostate | | | |
| | | cancer | | | |
| Porphyromonas | isolate 6C | isolated from | Porphyromonas | Porphyromonas | ERS5804565 |
| sp. nov. [symbol] | | prostatectomy | sp. nov. | bobii sp. nov. | (SEQ ID |
| | | specimen expressed | EMRHCC_6C | | NOs: 68-84) and |
| | | prostate secretion fluid | | | SAMEA8117582 |
| | | from a man with | | | |
| | | intermediate risk | | | |
| | | prostate cancer | | | |

TABLE 7
Detection of specific bacteria genera and species in clinical samples using qPCR assays
Bacterial species and genera detected in urine and prostate specimens using qPCR assays carried out
according to methods.

| | | Number of | Number of | Number of | |
|---|---|---|---|---|---|
| | Number of Urine | Prostate Secretion | Prostate Tissue | Periprostatic Fat | Number of Blood |
| | Samples Positive | Samples Positive | Samples Positive | Samples Positive | Samples Positive |
| Target Bacteria ID | (Total tested n = 49) | (Total tested n = 35) | (Total tested n = 70) | (Total tested n = 35) | (Total tested n = 20) |
| Fenollaria sp. nov. [symbol] | 32 | 1 | nd | nd | nd |
| Peptoniphilus sp. | 15 | 1 | 2 | 0 | 0 |
| nov. [symbol] | | | | | |
| Peptoniphilus harei | 39 | 4 | 9 | 2 | 0 |
| Varibaculum sp. | 10 | 2 | 6 | 0 | 0 |
| nov. [symbol] | | | | | |
| Porphyromonas sp. | 43 | 9 | 10 | 3 | 1 |
| Porphyromonas sp. | 3 | 6 | 0 | 0 | 0 |
| nov. [symbol] | | | | | |
| Porphyromonas | 9 | nd | nd | nd | nd |
| asaccharolytica | | | | | |
| Fusobacterium | 14 | 4 | 7 | 0 | 0 |
| nucleatum | | | | | |
| Propionimicrobium | 32 | 2 | 0 | 0 | 0 |
| lymphophilum | | | | | |
| Cutibacterium | 3 | 2 | 1 | 0 | 0 |
| acnes | | | | | |

nd: not determined

TABLE 8
Detection of specific bacteria genera and
species in clinical samples using qPCR assays

| | Genus or | | | |
|---|---|---|---|---|
| Target Bacteria | Species | | Primer F | Primer R |
| ID | Level Probe | Target | Sequence 5′-3′ | Sequence 5′-3′ |
| Fenollaria sp. | Species | RPL4 | GCGAACAAACGTCAAGGAAC | GCCTTTCCATTGAGGGCTTC |
| nov. [symbol] | | | (SEQ ID NO: 1) | (SEQ ID NO: 2) |
| Peptoniphilus sp. | Species | RPL4 | CACCGAAGACCAAGGCGTTA | GGTGCCGACCGTAGAAACTT |
| nov. [symbol] | | | (SEQ ID NO: 3) | (SEQ ID NO: 4) |
| Peptoniphilus | Species | RPL22 | GCGTTGATGAAGCCCTCTCTAT | ACCTTTAGCCTTAGGACGGAA |
| harei | | | (SEQ ID NO: 5) | (SEQ ID NO: 6) |
| Varibaculum sp. | Species | RPS3 | CGCTCGCAAACAGGTTGAAT | GGGCAGCATTTTCCGAAGC |
| nov. [symbol] | | | (SEQ ID NO: 7) | (SEQ ID NO: 8) |
| Porphyromonas sp. | Genus | 16S | AAGCGGAGGAACATGTGGTT | ACTTAAGCCGACACCTCACG |
| | | | (SEQ ID NO: 9) | (SEQ ID NO: 10) |
| Porphyromonas | Species | RPL22 | CCTACGTCGCCTCGTAAGAT | TGCGTCCGTTCTTCTCTTCC |
| sp. nov. [symbol] | | | (SEQ ID NO: 11) | (SEQ ID NO: 12) |
| Porphyromonas | Species | RPS3 | CGATCATACCTGGACGAGCC | TCGGCTACATACGTGGTTGG |
| asaccharolytica | | | (SEQ ID NO: 13) | (SEQ ID NO: 14) |
| Fusobacterium | Species | RPS3 | TCTGAATGGGCAGTTGAAGGA | AGCTTCCCCTCCTTCTTTCTT |
| nucleatum | | | (SEQ ID NO: 15) | (SEQ ID NO: 16) |
| Propionimicrobium | Species | RPL22 | ATGAGCCCGATGAAGGTTCG | CTACCGCAGAGGCAACTACC. |
| lymphophilum | | | (SEQ ID NO: 17) | (SEQ ID NO: 18) |
| Cutibacterium | Species | unique | GGATGACCTTGGTGGGGTAG | CACACAAATGGTGGTCACGG |
| acnes | | | (SEQ ID NO: 19) | (SEQ ID NO: 20) |

PCR primers used to detect each species and/or genera.

TABLE 9
16S Relative abundance of bacteria.
Bacteria at genus level are listed in order of relative abundance in Cluster 1 compared to the remaining
two clusters. Genera selected for further study based on relative abundance in Cluster 1 and statistical
significance included Fenollaria/Ezakiella, Peptoniphilus, Porphyromonas and Anaerococcus.

| | | | median relative | |
|---|---|---|---|---|
| | | median relative | abundance other | |
| Genus | P value | abundance Cluster 1 | Clusters | Adjusted P value |
| Ezakiella/Fenollaria | 0.000775889 | 20.22254341 | 0.144874846 | 0.030518295 |
| Prevotella | 0.115873648 | 7.020568159 | 27.31319324 | 0.564360451 |
| Peptoniphilus | 0.002319177 | 6.912546085 | 2.283346761 | 0.061069684 |
| Porphyromonas | 0.000624519 | 3.205810624 | 0.20272532 | 0.030518295 |
| Anaerococcus | 0.069616669 | 2.788885204 | 0.564219831 | 0.547651126 |
| Campylobacter | 0.010951604 | 1.663368086 | 0.191661906 | 0.176083542 |
| Negativicoccus | 2.99512E−05 | 0.982921136 | 0.004878525 | 0.003534239 |
| Varibaculum | 0.168219586 | 0.550157769 | 0.129890145 | 0.564360451 |
| Finegoldia | 0.571559692 | 0.496340506 | 0.552257236 | 0.741143337 |
| Mobiluncus | 0.018159881 | 0.300780346 | 0 | 0.238096213 |
| Propionimicrobium | 0.038439821 | 0.186991807 | 0.017344244 | 0.377991574 |
| Murdochiella | 0.011937867 | 0.031952799 | 0 | 0.176083542 |
| Aerococcus | 0.075283728 | 0 | 0.026495186 | 0.555217492 |
| Enterococcus | 0.002587698 | 0 | 0 | 0.061069684 |
| Escherichia-Shigella | 0.157143218 | 0 | 0 | 0.564360451 |
| Fusobacterium | 0.399399544 | 0 | 0.043136503 | 0.581841311 |
| Lawsonella | 0.068193095 | 0 | 0 | 0.547651126 |
| Psychrobacter | 0.094996393 | 0 | 0 | 0.564360451 |
| Treponema 2 | 0.032600998 | 0 | 0 | 0.349719796 |
| Veillonella | 0.063271445 | 0 | 0 | 0.547651126 |

TABLE 10
Anaerobic bacteria genera associated with poor prognosis prostate cancer included in the
Anaerobic Bacteria Biomarkers Set (ABBS).
Details of anaerobic bacteria genera included in ABBS,
including the phyla, class and order that the bacteria belong to. Details of the anaerobic bacteria species that
were isolated and identified in clinical samples belonging to genera included in ABBS.

| | | | | Novel Species | | |
|---|---|---|---|---|---|---|
| | | | | and known | Isolate IDs of | Other species |
| Anaerobic | | | | species isolated | novel species and | identified in |
| Bacteria | | | | by anaerobic | other species | clinical samples |
| Biomarker Set: | ABBS | ABBS | | culture | isolated | (165 sequence |
| ABBS bacteria | belonging to | belonging to | ABBS belonging | belonging to | belonging to | ID) belonging to |
| genera | Phylum | Class | to Order | ABBS genera | ABBS genera | ABBS genera |
| Fenollaria | Firmicutes | Clostridia | Clostridiales | Fenollaria sp. | Fenollaria sp. nov. | |
| | | | | nov. | (isolates: 24, 27A, | |
| | | | | | 27B, 29, 35) | |
| Peptoniphilus | Firmicutes | Clostridia | Clostridiales | Peptoniphilus | Peptoniphilus sp. | Peptoniphilus |
| | | | | sp. nov., | nov. (isolate: 23), | coxii |
| | | | | Peptoniphilus | Peptoniphilus | |
| | | | | harei | harei (isolates: 25, | |
| | | | | | 28, 33, 37, 38) | |
| Anaerococcus | Firmicutes | Clostridia | Clostridiales | Anaerococcus | Anaerococcus | Anaerococcus |
| | | | | prevotii | prevotii (isolates | lactolyticus |
| | | | | | 8B, 8S) | |
| Porphyromonas | Bacteroidetes | Bacteroidia | Bacteroidales | Porphyromonas | Porphyromonas | Porphyromonas |
| | | | | sp. nov., | sp. nov. (isolate: | bennonis |
| | | | | Porphyromonas | 6C), | |
| | | | | asaccharolytica | Porphyromonas | |
| | | | | | asaccharolytica | |
| | | | | | (isolates: 02_09, | |
| | | | | | 22_07) | |
| Fusobacterium | Fusobacteria | Fusobacteriia | Fusobacteriales | Fusobacterium | Fusobacterium | |
| | | | | nucleatum | nucleatum(isolate: | |
| | | | | | 02_15 (mixed) | |

TABLE 11
Clinical Data Associated with the urine microscopy cohort (n = 318).
Cohort is divided on the basis of microscopic bacterial detection in the urine.

| Characteristic | Bacteria Positive | | Bacteria Negative | | Total Dataset | |
|---|---|---|---|---|---|---|
| N | 168 | | 150 | | 318 | |
| Age, mean (median; IQR) | 69.44 | (69.5; 11) | 67.53 | (67; 11) | 68.55 | (68; 12) |
| PSA, mean (median; IQR) | 62.02 | (10.45; 15.65) | 11.81 | (7.1; 4.98) | 38.33 | (8.55; 8.95) |

Clinical Category, n

| | | | |
|---|---|---|---|
| Advanced | 16 | 2 | 18 |
| High Risk (D'amico) | 41 | 8 | 49 |
| Intermediate Risk (D'amico) | 56 | 26 | 82 |
| Low Risk (D'amico) | 6 | 11 | 17 |
| Other | 8 | 21 | 29 |
| Clinically Benign, no evidence | 11 | 28 | 39 |
| of cancer | | | |
| Raised PSA, Negative Biopsy | 30 | 54 | 84 |
| or unknown Gleason | | | |

Gleason Score, n

| 3 + 3 | 15 | | 13 | | 28 | |
|---|---|---|---|---|---|---|
| 3 + 4 | 38 | | 16 | | 54 | |
| 4 + 3 | 26 | | 8 | | 34 | |
| 4 + 4 | 9 | | 2 | | 11 | |
| 4 + 5 | 9 | | 4 | | 13 | |
| 5 + 4 | 8 | | 1 | | 9 | |
| 5 + 5 | 1 | | 0 | | 1 | |
| Cancer Specific Death, Yes (no; | 3 | (4, 161) | 0 | (1, 149) | 3 | (5; 310) |
| unknown) | | | | | | |
| Skeletal metastasis, Yes | 21 | (147) | 4 | (146) | 25 | (293) |
| (no/unknown) | | | | | | |

TABLE 12
Clinical data associated with the urine 16S sequencing cohort.
The groups indicate clusters found in FIG. 3. (3 samples known to have UTI and
originally included as UTI comparisons were removed from this analysis).

| Characteristic | Cluster 1 | | Cluster 2 | | Cluster 3 | |
|---|---|---|---|---|---|---|
| N | 22 | | 10 | | 14 | |
| Age, mean (median; IQR) | 70.45 | (73.00; 15.5) | 66.10 | (64.00; 10.75) | 68.00 | (65.50; 13.75) |
| PSA, mean (median; IQR) | 159.3 | (10.6; 19.6) | 100.27 | (6.10; 8.47) | 7.40 | (8.25; 4.5) |

Clinical Category, n

| | | | |
|---|---|---|---|
| Advanced | 4 | 1 | 0 |
| High Risk (D'amico) | 4 | 2 | 1 |
| Intermediate Risk (D'amico) | 1 | 4 | 5 |
| Low Risk (D'amico) | 0 | 1 | 0 |
| Other | 3 | 0 | 0 |
| Clinically Benign, no evidence | 5 | 1 | 4 |
| of cancer | | | |
| Raised PSA, Negative Biopsy | 5 | 1 | 4 |
| (or unknown gleason) | | | |

Gleason Score, n

| 3 + 3 | 0 | | 1 | | 0 | |
|---|---|---|---|---|---|---|
| 3 + 4 | 0 | | 3 | | 2 | |
| 4 + 3 | 3 | | 2 | | 3 | |
| 4 + 4 | 0 | | 0 | | 1 | |
| 4 + 5 | 0 | | 1 | | 0 | |
| 5 + 4 | 3 | | 0 | | 0 | |
| 5 + 5 | 0 | | 0 | | 0 | |
| Progression, n | 6 | | 4 | | 4 | |
| Skeletal Metastasis, n | 5 | | 1 | | 1 | |
| Leukocytes, positive (negative; | 8 | (10) | 2 | (5) | 5 | (7) |
| unknown) | | | | | | |
| Nitrates, positive (negative; | 1 | (17) | 0 | (7) | 0 | (13) |
| unknown) | | | | | | |

TABLE 13
Clinical data associated with the urine exosome RNA sequencing cohort.
Groups indicate the presence/absence of ABBS
(Ezakiella/Fenollaria, Peptoniphilus, Porphyromonas, Anaerococcus, Fusobacterium).
Progression/metastases events were determined by the following: appearance of prostate
cancer associated metastases on imaging, an increase in histological staging, prostate cancer associated
death or an increase in plasma PSA sufficient to affect the course of clinical treatment.

| | Samples positive for ABBS | | | | | |
|---|---|---|---|---|---|---|
| | genera associated with poor | | | | | |
| | prognosis: Anaerococcus, | | | | | |
| | Porphyromonas, | | Samples not positive for ABBS | | | |
| | Peptoniphilus, Fenollaria, | | genera associated with poor | | | |
| Characteristic | Fusobacterium | | prognosis | | Total Dataset | |
| N | 19 | | 21 | | 40 | |
| Age, mean (median; IQR) | 69.58 | (69.00; 12.5) | 67.05 | (66.00; 13.00) | 68.25 | (67.50; 14.25) |
| PSA, mean (median; IQR) | 11.24 | (7.70; 5.95) | 10.27 | (4.80, 9.00) | 10.73 | (6.90; 9.05) |

Clinical Category, n

| | | | |
|---|---|---|---|
| Advanced | 0 | 0 | 0 |
| High Risk (D'amico) | 6 | 6 | 12 |
| Intermediate Risk (D'amico) | 8 | 4 | 12 |
| Low Risk (D'amico) | 0 | 0 | 0 |
| Clinically Benign, no evidence | 5 | 11 | 16 |
| of cancer | | | |
| Raised PSA, Negative Biopsy | 0 | 0 | 0 |
| (or unknown gleason) | | | |

Gleason Score, n

| 3 + 3 | 0 | | 0 | | 0 | |
|---|---|---|---|---|---|---|
| 3 + 4 | 9 | | 4 | | 13 | |
| 4 + 3 | 3 | | 3 | | 6 | |
| 4 + 4 | 2 | | 0 | | 2 | |
| 4 + 5 | 2 | | 4 | | 6 | |
| 5 + 4 | 1 | | 0 | | 1 | |
| 5 + 5 | 0 | | 0 | | 0 | |
| Progression/metastasis, n | 11 | | 2 | | 13 | |
| Leukocytes, positive (negative; | 8 | (6) | 4 | (10) | 12 | (16; 12) |
| unknown) | | | | | | |
| Nitrates, positive (negative; | 0 | (14) | 0 | (14) | 0 | (28; 12) |
| unknown) | | | | | | |

TABLE 14
Clinical data associated with the ICGC whole genome sequence primary tumour samples
used for the targeted alignment approach.
Groups are split on the presence/absence of ABBS
(Ezakiella/Fenollaria, Peptoniphilus, Porphyromonas, Anaerococcus, Fusobacterium).
Only participants with clinical data are displayed.

| | Samples positive for ABBS | | | | | |
|---|---|---|---|---|---|---|
| | genera associated with poor | | | | | |
| | prognosis: Anaerococcus, | | | | | |
| | Porphyromonas, | | Samples not positive for ABBS | | | |
| | Peptoniphilus, Fenollaria, | | genera associated with poor | | | |
| Characteristic | Fusobacterium | | prognosis | | Total Dataset | |
| N | 26 | | 178 | | 204 | |
| Age, mean (median; IQR) | 63.54 | (62.00, 8.75) | 61.72 | (63.00, 10) | 61.96 | (62.50, 10) |
| PSA, mean (median; IQR) | 89.27 | (93.50, 55.5) | 81.45 | (87.50, 76.5) | 82.45 | (88.00, 76.25) |

Clinical Category, n

| | | | |
|---|---|---|---|
| Advanced | 1 | 1 | 2 |
| High Risk (D'amico) | 5 | 26 | 31 |
| Intermediate Risk (D'amico) | 16 | 120 | 136 |
| Low Risk (D'amico) | 3 | 31 | 34 |
| Clinically Benign, no evidence | 0 | 0 | 0 |
| of cancer | | | |
| Raised PSA, Negative Biopsy | 0 | 0 | 1 |
| (or Unknown Gleason) | | | |

Gleason Score, n

| 3 + 3 | 3 | | 49 | | 52 | |
|---|---|---|---|---|---|---|
| 3 + 4 | 14 | | 85 | | 99 | |
| 3 + 5 | 0 | | 3 | | 3 | |
| 4 + 3 | 5 | | 23 | | 28 | |
| 4 + 4 | 1 | | 5 | | 6 | |
| 4 + 5 | 1 | | 10 | | 11 | |
| 5 + 4 | 0 | | 3 | | 3 | |
| 5 + 5 | 0 | | 0 | | 0 | |
| Family History (first degree), | 5 | (1) | 48 | (16) | 53 | (17) |
| yes (no) | | | | | | |
| Cancer Specific Death, Yes (no) | 3 | (3) | 2 | (2) | 5 | (5) |

| | | | |
|---|---|---|---|
| Biochemical recurrence | 11 | 28 | 39 |
| metastasis | 1 | 4 | 6 |

TABLE 15
Bacterial genomes used to develop BWA alignment database for WGS data targeted approach.
The reference genomes were downloaded from NCBI refseq.
Assembly Name

| RefSeq Assembly | In House Isolate Assembly |
|---|---|
| Prevotella_bivia_DSM_20514 | Isolate_28_Peptoniphilus_duerdenii |
| Stenotrophomonas_maltophilia_K279a | Isolate_23_Peptoniphilus_sp. nov. |
| Fenollaria_massiliensis | Isolate_27B_Fenollaria sp. nov. |
| Propionimicrobium_lymphophilum_ACS.093.V.SCH5 | Isolate_39_Varibaculum sp. nov. |
| Staphylococcus_epidermidis_PM221 | Isolate_44b_Actinomyces_urogenitalis |
| Lysobacter_enzymogenes_strain_C3 | Isolate_3B_Streptococcus |
| Ralstonia_pickettii_12D | Isolate_4A_Staphylococcus_epidermidis |
| Escherichia_coli_str._K.12 | Isolate_5_Facklamia |
| Porphyromonas_somerae_DSM_23386 | Isolate_6B_Streptococcus |
| Anaerococcus_lactolyticus_ATCC_51172 | Isolate_3A_Streptococcus |
| Staphylococcus_aureus_subsp._ aureus_ST398 | Isolate_38_Peptoniphilus_harei |
| Prevotella_corporis_DSM_18810 | Isolate_6C_Porphyromonas sp. nov. |
| Propionimicrobium_sp._ BV2F7 | Isolate_6A_Streptococcus |
| Peptoniphilus_coxii_strain_DNF00729 | Isolate_4_Propionimicrobium_lymphophilum |
| Propionibacterium_acnes_ATCC_11828 | Isolate_5E_Cutibacterium_acnes |
| Actinotignum_urinale_DSM_15805 | Isolate_44a_Enterococcus_faecalis |
| Streptococcus_agalactiae_NEM316 | |
| Peptoniphilus_harei_ACS.146.V.Sch2b | |
| Fusobacterium_nucleatum_subsp._animalis_7_1 | |
| Enterococcus_faecalis_13.SD.W.01 | |
| Prevotella_timonensis_4401737 | |
| Sneathia_sanguinegens_strain_CCUG41628 | |
| Facklamia_hominis_ACS.120.V.Sch10 | |
| Acinetobacter_lwoffii_ATCC_9957 | |
| Mycoplasma_genitalium_G37 | |
| Aerococcus_urinae_ACS.120.V.Col10a | |
| Anaerococcus_hydrogenalis_DSM_7454 | |
| Actinobaculum_massiliense_ACS.171.V.Col2 | |
| Escherichia_coli_UMN026 | |
| Actinomyces_turicensis_ACS.279.V.Col4 | |
| Mycoplasma_hyorhinis_SK76 | |
| Mobiluncus_curtisii_ATCC_43063. | |
| Peptoniphilus_sp._BV3C26 | |
| Streptococcus_mitis_B6 | |
| Varibaculum_cambriense_DSM_15806 | |
| Actinomyces_neuii_BVS029A5 | |
| Fusobacterium_nucleatum_subsp._vincentii_3_1_36A2 | |
| Parvimonas_micra_A293 | |
| Streptococcus_anginosus_C238 | |
| Corynebacterium_pyruviciproducens_ATCC_BAA.1742 | |
| Prevotella_disiens_JCM_6334 | |
| Clostridiales_bacterium_9403326 | |
| Corynebacterium_pseudogenitalium_ATCC_33035 | |
| Anaerococcus_lactolyticus_S7.1.13 | |
| Actinobaculum_schaalii_strain_CCUG_27420. | |
| Pseudomonas_chlororaphis_HT66 | |
| Leishmania_major | |
| Toxoplasma_gondii_ME49 | |
| Anaerococcus_prevotii_ACS.065.V.Col13 | |
| Propionimicrobium_lymphophilum_DSM_4903 | |
| Porphyromonas_bennonis_DSM_23058 | |
| Anaerococcus_prevotii_DSM_20548 | |
| Enterococcus_faecalis_TX0630 | |
| Prevotella_buccalis_ATCC_35310 | |
| Finegoldia_magna_ATCC_53516 | |
| Fusobacterium_gonidiaformans_3.1.5R | |
| Corynebacterium_glucuronolyticum_ATCC_51867 | |
| Campylobacter_ureolyticus_RIGS_9880 | |

### Example 6—Quantitative Analysis of Bacteria Using qPCR-Based Approaches

We have confirmed the presence and assessed the prevalence of ABBS bacteria in urine in >300 clinical samples, bacteria including: Porphyromonas spp., Peptoniphilus spp. Fenollaria sporofastidiosus sp.nov., Fusobacterium nucleatum and Anaerococcus species (for complete list see Table 16 below). qPCR assays were designed and validated to detect species, including all four of the novel species, plus Peptoniphilus coxii, Peptoniphilus duerdenii, Porphyromonas bennonis, Anaerococcus prevotii Anaerococcus lactolyticus and co-occurring bacteria including Prevotella timonensis. The details of the PCR assays and primer pairs used to detect the bacteria are included in Table 17. The presence or absence of ABBS and co-occurring bacteria were assessed in more than 300 urine samples using the genera- or species-specific qPCR assays (Table 16).

TABLE 16
Detection of specific bacteria genera and species in urine samples with
genera- and species-specific qPCR assays. (♦ = novel species)

| | Number of | |
|---|---|---|
| | urine | % |
| | samples | urine samples |
| | positive | positive for |
| Target bacteria ID | (Total n = 304) | bacterial species |
| Fenollaria sp. nov. ♦ | 64 | 21.1% |
| Peptoniphilus sp. nov. ♦ | 25 | 8.2% |
| Peptoniphilus coxii | 61 | 20% |
| Peptoniphilus duerdenii | 53 | 17.4% |
| Peptoniphilus harei | 169 | 55.5% |
| Porphyromonas sp. (genus level) | 243 | 79.9% |
| Porphyromonas sp. nov. ♦ | 3 | 1% |
| Porphyromonas asaccharolytica | 20 | 6.58% |
| Porphyromonas bennonis | 49 | 16.1% |
| Anaerococcus prevotii | 102 | 33.5% |
| Anaerococcus lactolyticus | 130 | 42.7% |
| Fusobacterium nucleatum | 36 | 11.8% |
| Varibaculum sp. nov. ♦ | 40 | 113.2% |
| Prevotella sp. (genus level) | 210 | 69.1% |
| Prevotella timonensis | 104 | 34.2% |
| Propionimicrobium lymphophilum | 120 | 39.5% |
| Campylobacter ureolyticus | 79 | 26% |
| Human DNA | 304 | |

TABLE 17
Detection of specific bacteria genera and
species in urine using qPCR assays.

| Target | Genus or | | | |
|---|---|---|---|---|
| Bacteria | Species | | Primer F | Primer R |
| ID | Level Assay | Target | Sequence 5′-3′ | Sequence 5′-3′ |
| Peptoniphilus | Species | RPL22 | AGTTCTTCACTCCGCTGTCG | CGCCATACCCTTTGCTTTGG |
| coxii | | | (SEQ ID NO: 85) | (SEQ ID NO: 86) |
| Peptoniphilus | Species | RPL22 | TCGTGGCAAAAGCGTTGAAG | GTTGGTCCGTCATTTGCGTA |
| duerdenii | | | (SEQ ID NO: 87) | (SEQ ID NO: 88) |
| Porphyromonas | Species | RPL32 | ACTCACGACGTAGCCAAGAC | TCCACCTCTACAGCGATCTGA |
| bennonis | | | (SEQ ID NO: 89) | (SEQ ID NO: 90) |
| Anaerococcus | Species | RPL4 | GCAAATACAACTCCACCGCC | TGCGAAAACTCGTTCAGAGGT |
| prevotii | | | (SEQ ID NO: 91) | (SEQ ID NO: 92) |
| Anaerococcus | Species | RPL22 | CGCTGAAAACAACAACGGACT | CCGCTAACTCTACGCCGATA |
| lactolyticus | | | (SEQ ID NO: 93) | (SEQ ID NO: 94) |
| Prevotella sp. | Genus | 16S | GCACGGTAAACGATGGATGC | TTCAAGCCCGGGTAAGGTTC |
| | | | (SEQ ID NO: 95) | (SEQ ID NO: 96) |
| Prevotella | Species | RPL22 | GAGCAAGAAAGCATATAGCGGC | TCGCAATACTCCAAGGGCAC |
| timonensis | | | (SEQ ID NO: 97) | (SEQ ID NO: 98) |

Details of qPCR assays carried out according to methods.

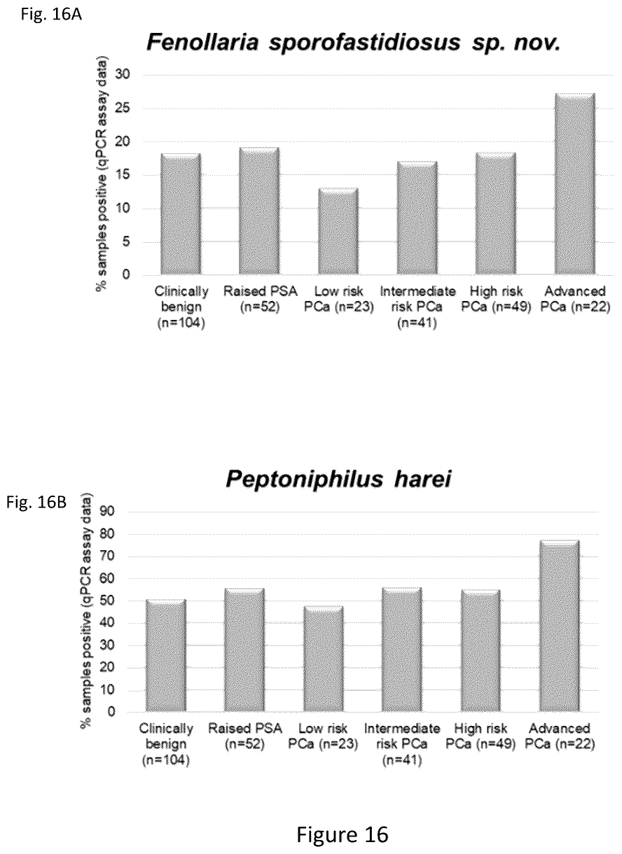

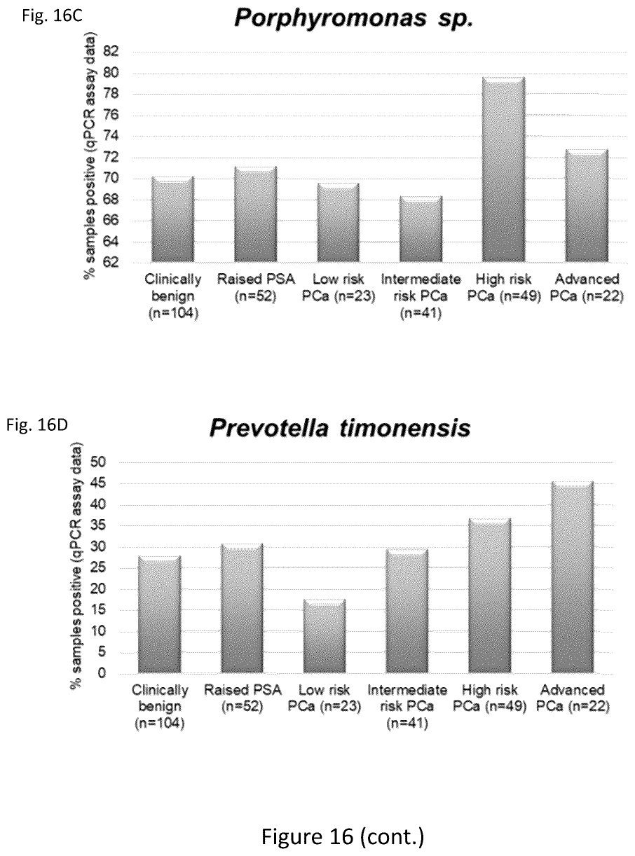

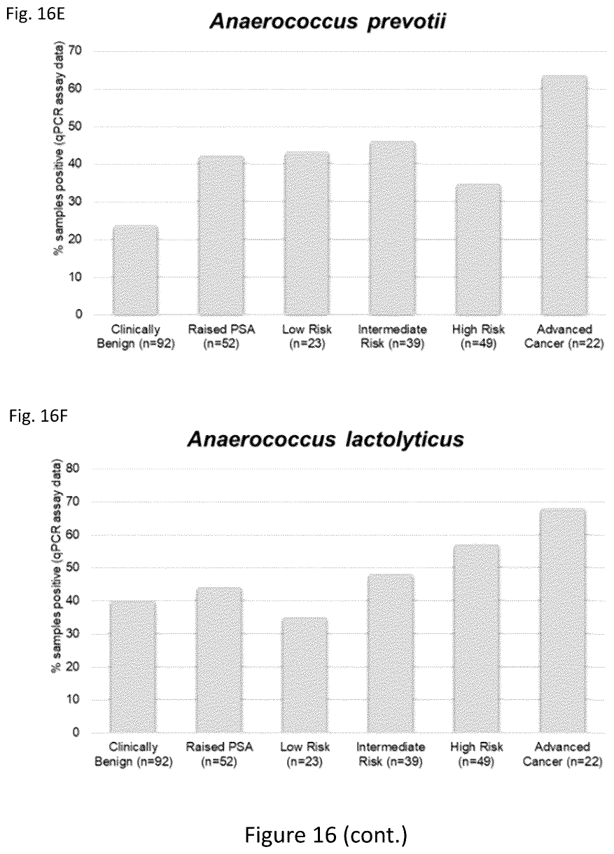

Several bacteria genera and species were present in >50% of urine samples analysed from patients attending the urology PSA clinic, including Porphyromonas sp., Prevotella sp. and Peptoniphilus harei. The four novel species were detected in urine samples, with Porphyromonas sp. nov only in <z1% of samples, Peptoniphilus sp. nov. in 8% of the samples analysed, Varibaculum sp. nov. in 13% and F. Sporofastidiosus sp. nov in 21% of urine samples. The results for several of the qPCR assays for screening the presence and absence of bacteria in the urine samples across the clinical categories are shown below in FIG. 16. Investigation into the presence of bacteria detected by qPCR and clinical category at baseline indicates that there is a trend towards an increased percentage of samples positive for certain bacterial species/genera in high risk or advanced clinical samples (for example see Fenollaria sporofastidiosus sp. nov., Peptoniphilus harei, Prevotella timonensis, Porphyromonas sp., Anaerococcus lactolyticus and Anaerococcus prevotii in FIG. 16). Prevotella timonesis may be a particularly useful bactertial species in detecting and distinguishing high risk prostate cancer groups in clinical samples.

Genomic sequence details of Prevotella timonensis are available in established sequence databases and are disclosed herein as SEQ ID NOs: 99-120. The methods described herein may use a nucleotide primer that is substantially complementary or reverse complementary to a corresponding length portion of any one of SEQ ID NOs: 99-120.

TABLE 18
Sequence deposit details of Prevotella timonensis

| GenBank Accession | RefSeq Accession | Title |
|---|---|---|
| GCA_002894165.1 | GCF_002894165.1 | Prevotella |
| (SEQ ID NOs: 99-120) | | timonensis |

### Example 7—ABBS Genera Detection in Prostate Tumor Whole Genome Sequencing Samples

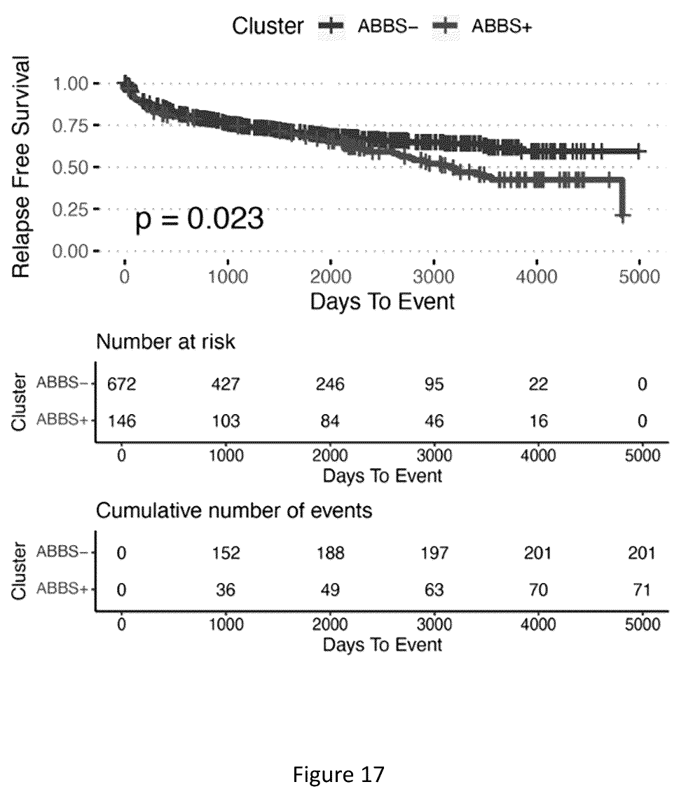

SEPATH [63] was ran on N=2,176 prostate tumourwhole genome sequencing samples from the pan-prostate cancer group (http://panprostate.org). Participants were filtered to those containing followup data (N=818). Participants with evidence for at least one ABBS genera (N=146, see FIG. 17; blue, as determined by a minimum of 10 taxonomically classified sequencing reads by Kraken for at least one of the genera: Fenollaria, Ezakiella, Peptoniphilus, Porphyromonas, Anaerococcus, Fusobacterium) were compared to those that had no evidence of ABBS genera (N=672, see FIG. 17; red). There was a statistically significant difference between the groups in terms of relapse-free survival (P=0.023, log-rank test), with the ABBS group demonstrating a poorer prognosis.

### Survival Analysis

Survival analysis was carried out in R (version 4.2.1). Models were fit using the survfit function (survival package, version 3.5.0) and plotted using ggsurvplot (survminer package, version 0.4.9).

Additional examples of the presently described method and system embodiments are suggested according to the structures and techniques described herein. Other non-limiting examples may be configured to operate separately, or can be combined in any permutation or combination with any one or more of the other examples provided above or throughout the present disclosure.

All publications, patents and patent applications referred to herein are incorporated by reference in their entirety to the same extent as if each individual publication, patent or patent application was specifically and individually indicated to be incorporated by reference in its entirety.

### Embodiments

Further embodiments of the present invention are described below:

1. A method of detecting the presence of one or more bacteria selected from the genera Fenollaria, Ezakiella, Peptoniphilus, Porphyromonas, Anaerococcus, Varibaculum and Fusobacterium in a biological sample from the patient.

2. A method of detecting the presence of one or more bacteria selected from the genera Fenollaria, Peptoniphilus, Porphyromonas, Anaerococcus and Fusobacterium in a biological sample from the patient.

3. The method according to embodiment 1 or embodiment 2, wherein the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

4. The method according to any one of embodiments 1 to 3, wherein the one or more bacteria are selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

5. The method according to any one of embodiments 1 to 3, wherein the one or more bacteria are selected from: Fenollaria sporofastidiosus, Peptoniphilus rachelemmaiella, Anaerococcus prevotii, Porphyromonas bobii, Porphyromonas asaccharolytica and Fusobacterium nucleatum.

6. The method according to any one of embodiments 1 to 3, wherein the one or more bacteria are selected from: Fenollaria sporofastidiosus, Peptoniphilus harei, Anaerococcus prevotii, Porphyromonas bobii, Porphyromonas asaccharolytica and Fusobacterium nucleatum.

7. The method according to any one of embodiments 1 to 3, wherein the one or more bacteria are selected from: Fenollaria sporofastidiosus, Peptoniphilus rachelemmaiella, Peptoniphilus harei, Anaerococcus prevotii, Porphyromonas asaccharolytica and Fusobacterium nucleatum.

8. The method according to any one of embodiments 1 to 3, wherein the one or more bacteria are selected from: Fenollaria sporofastidiosus, Peptoniphilus rachelemmaiella, Peptoniphilus harei, Anaerococcus prevotii, Porphyromonas bobii and Fusobacterium nucleatum.

9. The method according to any one of embodiments 1 to 3, wherein the one or more bacteria are selected from: Fenollaria sporofastidiosus, Peptoniphilus rachelemmaiella, Anaerococcus prevotii, Porphyromonas bobii and Fusobacterium nucleatum.

10. The method according to any one of embodiments 1 to 3, wherein the one or more bacteria are selected from: Fenollaria sporofastidiosus, Peptoniphilus rachelemmaiella, Anaerococcus prevotii, Porphyromonas asaccharolytica and Fusobacterium nucleatum.

11. The method according to any one of embodiments 1 to 3, wherein the one or more bacteria are selected from: Fenollaria sporofastidiosus, Peptoniphilus harei, Anaerococcus prevotii, Porphyromonas bobii and Fusobacterium nucleatum.

12. The method according to any one of embodiments 1 to 3, wherein the one or more bacteria are selected from: Fenollaria sporofastidiosus, Peptoniphilus harei, Anaerococcus prevotii, Porphyromonas asaccharolytica and Fusobacterium nucleatum.

13. The method according to any one of embodiments 1 to 12, wherein the method comprises detecting the presence of at least 1, 2, 3, 4, 5, 6, 7, 8, 9 or 10 different bacteria.

14. The method according to any one of embodiments 1 to 13, wherein the method comprises detecting all of the species and/or genera recited in a given list.

15. The method according to embodiment 1 or embodiment 2, wherein the method comprises detecting each of Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

16. The method according to any one of embodiments 1 to 15, wherein the method comprises detecting each of Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

17. The method according to any one of embodiments 4 to 14 or 16, further comprising detecting the presence of one or more bacteria selected from Peptoniphilus coxii, Anaerococcus lactolyticus and Porphyromonas bennonis.

18. The method according to any one of embodiments 1 to 17, further comprising detecting the presence of Varibaculum prostatecancerukia.

19. The method according to any one of embodiments 1 to 18, wherein one or more of the bacteria are selected from: Fenollaria sporofastidiosus (identified by ECACC accession number 21092201 or DSMZ accession number DSM 34056), Peptoniphilus rachelemmaiella (identified by ECACC accession number 21092202 or DSMZ accession number DSM 34055) and Porphyromonas bobii (identified by ECACC accession number 21092204 or DSMZ accession number DSM 34063).

20. The method according to any one of embodiments 1 to 19, wherein the bacteria is Varibaculum prostatecancerukia (identified by ECACC accession number 21092203 or DSMZ accession number DSM 34057).

21. The method according to any preceding embodiment, wherein the biological sample is processed prior to determining the presence of the one or more bacteria in the biological sample.

22. The method according to any preceding embodiment, wherein determining the presence of the one or more bacteria comprises extracting a nucleic acid from the biological sample.

23. The method according to any preceding embodiment, wherein determining the presence of the one or more bacteria comprises extracting bacterial DNA from the biological sample.

24. The method according to any preceding embodiment, wherein determining the presence of the one or more bacteria comprises extracting bacterial RNA from the biological sample.

25. The method according to any preceding embodiment, wherein determining the presence of the one or more bacteria comprises extracting one or more bacterial proteins from the biological sample.

26. The method according to any preceding embodiment, wherein determining the presence of the one or more bacteria comprises extracting one or more bacteria-specific carbohydrates from the biological sample.

27. The method according to any preceding embodiment, wherein the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample).

28. The method according to any preceding embodiment wherein the biological sample is a urine sample.

29. The method according to any preceding embodiment, wherein the presence of the one or more bacteria is determined using quantitative polymerase chain reaction (qPCR) or a probe-based detection assay (such as NanoString®).

30. The method according to any preceding embodiment, wherein the presence of the one or more bacteria is determined using quantitative polymerase chain reaction (qPCR) or a probe-based detection assay (such as NanoString®) wherein the qPCR or probe-based detection assay is conducted on one or more of bacterial nucleic acids in the biological sample.

31. The method according to any preceding embodiment, wherein determining the presence of the one or more bacteria comprises detecting one or more bacterial proteins in a sample.

32. The method according to any preceding embodiment, wherein determining the presence of the one or more bacteria comprises detecting one or more bacterial-specific carbohydrates.

33. The method according to any preceding embodiment, wherein the presence of the one or more bacteria is determined using matrix-assisted laser desorption/ionization-time of flight mass spectrometry (MALDI-TOF).

34. The method according to any one of embodiments 1 to 30, wherein the presence of the one or more bacteria is determined using quantitative polymerase chain reaction (qPCR) or a probe-based detection assay (such as NanoString®) using one or more primers comprising a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

35. The method according to any one of embodiments 1 to 30, wherein the presence of the one or more bacteria is determined using quantitative polymerase chain reaction (qPCR) or a probe-based detection assay (such as NanoString®) using one or more primers consisting of a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

36. A method of providing a cancer prognosis in a patient based on the presence of one or more bacteria in a biological sample from the patient, comprising:
(a) detecting the presence of one or more bacteria selected from the genera Fenollaria, Ezakiella, Peptoniphilus, Porphyromonas, Anaerococcus, Varibaculum and Fusobacterium in a biological sample from the patient, and
(b) providing a prognosis for the patient, wherein the patient has a poor prognosis if one or more of the bacteria are present in the biological sample.

37. A method of providing a cancer prognosis in a patient based on the presence of one or more bacteria in a biological sample from the patient, comprising:
(a) detecting the presence of one or more bacteria selected from the genera Fenollaria, Peptoniphilus, Porphyromonas, Anaerococcus and Fusobacterium in a biological sample from the patient, and
(b) providing a prognosis for the patient, wherein the patient has a poor prognosis if one or more of the bacteria are present in the biological sample.

38. The method according to embodiment 36 or embodiment 37, wherein the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

39. The method according to any one of embodiments 36 to 38, wherein the one or more bacteria are selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

40. The method according to any one of embodiments 36 to 38, wherein the one or more bacteria are selected from: Fenollaria sporofastidiosus, Peptoniphilus rachelemmaiella, Anaerococcus prevotii, Porphyromonas bobii, Porphyromonas asaccharolytica and Fusobacterium nucleatum.

41. The method according to any one of embodiments 36 to 38, wherein the one or more bacteria are selected from: Fenollaria sporofastidiosus, Peptoniphilus harei, Anaerococcus prevotii, Porphyromonas bobii, Porphyromonas asaccharolytica and Fusobacterium nucleatum.

42. The method according to any one of embodiments 36 to 38, wherein the one or more bacteria are selected from: Fenollaria sporofastidiosus, Peptoniphilus rachelemmaiella, Peptoniphilus harei, Anaerococcus prevotii, Porphyromonas asaccharolytica and Fusobacterium nucleatum.

43. The method according to any one of embodiments 36 to 38, wherein the one or more bacteria are selected from: Fenollaria sporofastidiosus, Peptoniphilus rachelemmaiella, Peptoniphilus harei, Anaerococcus prevotii, Porphyromonas bobii and Fusobacterium nucleatum.

44. The method according to any one of embodiments 36 to 38, wherein the one or more bacteria are selected from: Fenollaria sporofastidiosus, Peptoniphilus rachelemmaiella, Anaerococcus prevotii, Porphyromonas bobii and Fusobacterium nucleatum.

45. The method according to any one of embodiments 36 to 38, wherein the one or more bacteria are selected from: Fenollaria sporofastidiosus, Peptoniphilus rachelemmaiella, Anaerococcus prevotii, Porphyromonas asaccharolytica and Fusobacterium nucleatum.

46. The method according to any one of embodiments 36 to 38, wherein the one or more bacteria are selected from: Fenollaria sporofastidiosus, Peptoniphilus harei, Anaerococcus prevotii, Porphyromonas bobii and Fusobacterium nucleatum.

57. The method according to any one of embodiments 36 to 38, wherein the one or more bacteria are selected from: Fenollaria sporofastidiosus, Peptoniphilus harei, Anaerococcus prevotii, Porphyromonas asaccharolytica and Fusobacterium nucleatum.

48. The method according to any one of embodiments 36 to 47, wherein the method comprises detecting the presence of at least 1, 2, 3, 4, 5, 6, 7, 8, 9 or 10 different bacteria.

49. The method according to any one of embodiments 36 to 48, wherein the method comprises detecting all of the species recited in a given list.

50. The method according to any one of embodiments 36 to 38, wherein the method comprises detecting each of Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

51. The method according to any one of embodiments 36, 37 or 39, wherein the method comprises detecting each of Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

52. The method according to any one of embodiments 36 to 49 or 51, further comprising detecting the presence of one or more bacteria selected from Peptoniphilus coxii, Anaerococcus lactolyticus and Porphyromonas bennonis.

53. The method according to any one of embodiments 36 to 52, further comprising detecting the presence of Varibaculum prostatecancerukia.

54. The method according to any one of embodiments 36 to 53, wherein one or more of the bacteria are selected from: Fenollaria sporofastidiosus (identified by ECACC accession number 21092201 or DSMZ accession number DSM 34056), Peptoniphilus rachelemmaiella (identified by ECACC accession number 21092202 or DSMZ accession number DSM 34055) and Porphyromonas bobii (identified by ECACC accession number 21092204 or DSMZ accession number DSM 34063).

55. The method according to any one of embodiments 36 to 54, wherein the bacteria is Varibaculum prostatecancerukia (identified by ECACC accession number 21092203 or DSMZ accession number DSM 34057).

56. The method according to any one of embodiments 36 to 55, wherein a poor prognosis is associated with an increased risk of elevated rates of metastases.

57. The method according to any one of embodiments 36 to 55, wherein a poor prognosis is associated with a progression of the cancer stage.

58. The method according to any one of embodiments 36 to 55, wherein a poor prognosis is associated with an increase in prostate-specific antigen (PSA) and/or PSA failure.

59. The method according to any one of embodiments 36 to 55, wherein a poor prognosis is associated with an increased cancer risk score, optionally wherein the risk score is a Gleason risk score.

60. The method according to any one of embodiments 36 to 59, wherein the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample).

61. The method according to any one of embodiments 36 to 60, wherein the biological sample is a urine sample.

62. The method according to any one of embodiments 36 to 61, wherein the method is used to determine whether a patient should be biopsied.

63. The method according to any one of embodiments 36 to 62, wherein the biological sample is processed prior to determining the presence of the one or more bacteria in the biological sample.

64. The method according to any one of embodiments 36 to 63, wherein determining the presence of the one or more bacteria comprises extracting a nucleic acid from the biological sample.

65. The method according to any one of embodiments 36 to 64, wherein determining the presence of the one or more bacteria comprises extracting bacterial DNA from the biological sample.

66. The method according to any one of embodiments 36 to 64, wherein determining the presence of the one or more bacteria comprises extracting bacterial RNA from the biological sample.

67. The method according to any one of embodiments 36 to 64, wherein determining the presence of the one or more bacteria comprises detecting one or more bacterial proteins in a sample.

68. The method according to any one of embodiments 36 to 64, wherein determining the presence of the one or more bacteria comprises detecting one or more bacterial-specific carbohydrates.

69. The method according to any one of embodiments 36 to 66, wherein the presence of the one or more bacteria is determined using quantitative polymerase chain reaction (qPCR) or a probe-based detection assay (such as NanoString®).

70. The method according to any one of embodiments 36 to 66 or 69, wherein the presence of the one or more bacteria is determined using quantitative polymerase chain reaction (qPCR) or a probe-based detection assay (such as NanoString®) using one or more primers comprising a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

71. The method according to any one of embodiments 36 to 66 or 69, wherein the presence of the one or more bacteria is determined using quantitative polymerase chain reaction (qPCR) or a probe-based detection assay (such as NanoString®) using one or more primers consisting of a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

72. The method according to any preceding embodiment wherein the sample is from a human.

73. A method of treating a cancer in a patient in need thereof, comprising providing a prognosis of a patient's cancer using a method as defined in any one of embodiments 36 to 72, and administering to the patient a therapy for treating cancer.

74. A method of treating cancer in a patient in need thereof, wherein the prognosis of the patient has been determined according to a method as defined in any one of embodiments 36 to 72, comprising administering to the patient a therapy for treating cancer.

75. The method according to any one of embodiments 32 to 67, wherein the cancer is selected from the list: Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Central Nervous System (Brain Cancer), Basal Cell Carcinoma, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors (Lung Cancer), Burkitt Lymphoma—see Non-Hodgkin Lymphoma, Carcinoid Tumor (Gastrointestinal), Cardiac (Heart) Tumors, Medulloblastoma and Other CNS Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Embryonal Tumors, Medulloblastoma, Endometrial Cancer, Ependymoma, Esophageal Cancer, Esthesioneuroblastoma, Ewing Sarcoma (Bone Cancer), Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Heart Tumors, Hepatocellular (Liver) Cancer, Histiocytosis, Langerhans Cell Hodgkin Lymphoma, Hypopharyngeal Cancer, Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Melanoma, Intraocular (Eye), Merkel Cell Carcinoma, Mesothelioma, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, Acute (AML), Myeloproliferative Neoplasms, Chronic Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma, Childhood Rhabdomyosarcoma (Soft Tissue Sarcoma), Childhood Vascular Tumors (Soft Tissue Sarcoma), Ewing Sarcoma (Bone Cancer), Osteosarcoma (Bone Cancer), Soft Tissue Sarcoma, Uterine Sarcoma, Sezary Syndrome, Skin Cancer, Small Cell Lung Cancer, Small Intestine Cancer, Squamous Cell Carcinoma, Squamous Neck Cancer with Occult Primary, Metastatic, Stomach (Gastric) Cancer, T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Ureter and Renal Pelvis, Transitional Cell Cancer (Kidney (Renal Cell) Cancer, Urethral Cancer, Uterine Cancer, Vaginal Cancer, Vascular Tumors, Vulvar Cancer, Wilms Tumor, Myxofibrosarcoma, Leiomyosarcoma, Pleomorphic Sarcoma and Hepatopancreatobiliary cancer.

76. The method according to any one of embodiments 36 to 75, wherein the cancer is prostate cancer.

77. The method according to any one of embodiments 73 to 76, wherein the therapy for cancer comprises surgery, brachytherapy, active surveillance, chemotherapy, hormone therapy, immunotherapy and/or radiotherapy.

78. The method according to any one of embodiments 73 to 76, wherein the therapy for cancer comprises chemotherapy, hormone therapy and/or immunotherapy.

79. The method according to embodiment 77 or embodiment 78, wherein the chemotherapy comprises administration of one or more agents selected from the list: abiraterone acetate, apalutamide, bicalutamide, cabazitaxel, bicalutamide, degarelix, docetaxel, leuprolide acetate, enzalutamide, apalutamide, flutamide, goserelin acetate, mitoxantrone, nilutamide, sipuleucel T, radium 223 dichloride and docetaxel.

80. The method according to any one of embodiments 73 to 77, wherein the therapy for cancer comprises resection of all or part of a tumour.

81. The method according to any one of embodiments 73 to 77, wherein the cancer is prostate cancer and the therapy for cancer comprises resection of all or part of the prostate gland and/or all or part of a prostate tumour.

82. A nucleotide primer comprising a sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

83. The nucleotide primer according to embodiment 82, consisting of a sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

84. A kit for determining the presence of one or more bacteria in a biological sample, comprising means for detecting one or more bacteria selected from the genera Fenollaria, Ezakiella, Peptoniphilus, Porphyromonas, Anaerococcus, Varibaculum and Fusobacterium in a biological sample.

85. A kit for determining the presence of one or more bacteria in a biological sample, comprising means for detecting one or more bacteria selected from the genera Fenollaria, Peptoniphilus, Porphyromonas, Anaerococcus and Fusobacterium in a biological sample.

86. The kit according to embodiment 84 or embodiment 85, wherein the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

87. The kit according to any one of embodiments 84 to 86, wherein the one or more bacteria are selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

88. The kit according to any one of embodiments 84 to 86, wherein the one or more bacteria are selected from: Fenollaria sporofastidiosus, Peptoniphilus rachelemmaiella, Anaerococcus prevotii, Porphyromonas bobii, Porphyromonas asaccharolytica and Fusobacterium nucleatum.

89. The kit according to any one of embodiments 84 to 86, wherein the one or more bacteria are selected from: Fenollaria sporofastidiosus, Peptoniphilus harei, Anaerococcus prevotii, Porphyromonas bobii, Porphyromonas asaccharolytica and Fusobacterium nucleatum.

90. The kit according to any one of embodiments 84 to 86, wherein the one or more bacteria are selected from: Fenollaria sporofastidiosus, Peptoniphilus rachelemmaiella, Peptoniphilus harei, Anaerococcus prevotii, Porphyromonas asaccharolytica and Fusobacterium nucleatum.

91. The kit according to any one of embodiments 84 to 86, wherein the one or more bacteria are selected from: Fenollaria sporofastidiosus, Peptoniphilus rachelemmaiella, Peptoniphilus harei, Anaerococcus prevotii, Porphyromonas bobii and Fusobacterium nucleatum.

92. The kit according to any one of embodiments 84 to 86, wherein the one or more bacteria are selected from: Fenollaria sporofastidiosus, Peptoniphilus rachelemmaiella, Anaerococcus prevotii, Porphyromonas bobii and Fusobacterium nucleatum.

93. The kit according to any one of embodiments 84 to 86, wherein the one or more bacteria are selected from: Fenollaria sporofastidiosus, Peptoniphilus rachelemmaiella, Anaerococcus prevotii, Porphyromonas asaccharolytica and Fusobacterium nucleatum.

94. The kit according to any one of embodiments 84 to 86, wherein the one or more bacteria are selected from: Fenollaria sporofastidiosus, Peptoniphilus harei, Anaerococcus prevotii, Porphyromonas bobii and Fusobacterium nucleatum.

95. The kit according to any one of embodiments 84 to 86, wherein the one or more bacteria are selected from: Fenollaria sporofastidiosus, Peptoniphilus harei, Anaerococcus prevotii, Porphyromonas asaccharolytica and Fusobacterium nucleatum.

96. The kit according to any one of embodiments 84 to 95, wherein the kit comprises means for detecting the presence of at least 1, 2, 3, 4, 5, 6, 7, 8, 9 or 10 different bacteria.

97. The kit according to any one of embodiments 84 to 96, wherein the kit comprises means for detecting all of the species recited in a given list.

98. The kit according to any one of embodiments 84 to 86, wherein the kit comprises means for detecting each of Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

99. The kit according to any one of embodiments 84, 85 or 87, wherein the kit comprises means for detecting each of Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

100. The kit according to any one of embodiments 84 to 97 or 99, further comprising means for detecting the presence of one or more bacteria selected from Peptoniphilus coxii, Anaerococcus lactolyticus and Porphyromonas bennonis.

101. The kit according to any one of embodiments 84 to 100, further comprising means for detecting the presence of Varibaculum prostatecancerukia.

102. The kit according to any one of embodiments 84 to 101, wherein one or more of the bacteria are selected from: Fenollaria sporofastidiosus (identified by ECACC accession number 21092201 or DSMZ accession number DSM 34056), Peptoniphilus rachelemmaiella (identified by ECACC accession number 21092202 or DSMZ accession number DSM 34055) and Porphyromonas bobii (identified by ECACC accession number 21092204 or DSMZ accession number DSM 34063).

103. The kit according to any one of embodiments 84 to 102, wherein the bacteria is Varibaculum prostatecancerukia (identified by ECACC accession number 21092203 or DSMZ accession number DSM 34057).

104. The kit according to any one of embodiments 84 to 103, wherein the means for detecting is a biosensor or specific binding molecule.

105. The kit according to embodiment 104, wherein the biosensor is an electrochemical, electronic, piezoelectric, gravimetric, pyroelectric biosensor, ion channel switch, evanescent wave, surface plasmon resonance or biological biosensor.

106. The kit according to embodiment 104 or 105 wherein the biosensor is an antigen-specific binding molecule capable of specifically binding to the one or more bacteria, optionally wherein the antigen-specific binding molecule is an antibody or fragment thereof.

107. The kit according to any one of embodiments 84 to 103, wherein the means for detecting is quantitative polymerase chain reaction (qPCR) or a probe-based detection assay (such as NanoString®).

108. The kit according to embodiment 107, wherein the means for detecting is quantitative polymerase chain reaction (qPCR) or a probe-based detection assay (such as NanoString®) and the kit comprises one or more primers comprising a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

109. The kit according to embodiment 107 or 108, wherein the means for detecting is quantitative polymerase chain reaction (qPCR) or a probe-based detection assay (such as NanoString®) and the kit comprises one or more primers consisting of a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

110. A cell culture comprising bacteria Varibaculum prostatecancerukia identified by ECACC deposit number 21092203 or DSMZ accession number DSM 34057.

111. A cell culture comprising bacteria Fenollaria sporofastidiosus identified by ECACC deposit number 21092201 or DSMZ accession number DSM 34056.

112. A cell culture comprising bacteria Peptoniphilus rachelemmaiella identified by ECACC deposit number 21092202 or DSMZ accession number DSM 34055.

113. A cell culture comprising bacteria Porphyromonas bobii identified by ECACC deposit number 21092204 or DSMZ accession number DSM 34063.

114. Use of a cell culture according to any one of embodiments 110 to 113 in providing a prognosis of cancer in a patient.

115. A computer apparatus configured to perform a method according to any one of embodiments 1 to 72 or, a computer readable medium programmed to perform a method according to any one of embodiments 1 to 72.

116. The method according to any one of embodiments 1 to 4, wherein one or more of the bacteria comprise 1, 2, 3, 4, 5, 6 or 7 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

117. The method according to any one of embodiments 1 to 4, wherein one or more of the bacteria comprise 2 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

118. The method according to any one of embodiments 1 to 4, wherein one or more of the bacteria comprise 3 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

119. The method according to any one of embodiments 1 to 4, wherein one or more of the bacteria comprise 4 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

120. The method according to any one of embodiments 1 to 4, wherein one or more of the bacteria comprise 5 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

121. The method according to any one of embodiments 1 to 4, wherein one or more of the bacteria comprise 6 bacteria selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

122. The method according to any one of embodiments 1 to 4, wherein one or more of the bacteria comprise all 7 bacteria Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

123. A nucleotide primer comprising a sequence between 5 and 25 nucleotides in length that is substantially complementary or reverse complementary to a corresponding length portion of SEQ ID NO:25.

124. The nucleotide primer according to embodiment 123, wherein the primer is capable of detecting the presence of Varibaculum prostatecancerukia in a biological sample.

125. A nucleotide primer comprising a sequence between 5 and 25 nucleotides in length that is substantially complementary or reverse complementary to a corresponding length portion of SEQ ID NO:26.

126. The nucleotide primer according to embodiment 125, wherein the primer is capable of detecting the presence of Fenollaria sporofastidiosus in a biological sample.

127. A nucleotide primer comprising a sequence between 5 and 25 nucleotides in length that is substantially complementary or reverse complementary to a corresponding length portion of a sequence selected from the list consisting of SEQ ID NOs:27-67.

128. The nucleotide primer according to embodiment 127, wherein the primer is capable of detecting the presence of Peptoniphilus rachelemmaiella in a biological sample.

129. A nucleotide primer comprising a sequence between 5 and 25 nucleotides in length that is substantially complementary or reverse complementary to a corresponding length portion of a sequence selected from the list consisting of SEQ ID NOs:68-84.

130. The nucleotide primer according to embodiment 129, wherein the primer is capable of detecting the presence of Porphyromonas bobii in a biological sample.

131. The method according to any one of embodiments 1 to 72, wherein the presence of the one or more bacteria is determined using quantitative polymerase chain reaction (qPCR) or a probe-based detection assay (such as NanoString®) using one or more primers according to embodiments 123 to 130.

132. The kit according to any one of embodiments 84 to 109, wherein the means for detecting is quantitative polymerase chain reaction (qPCR) or a probe-based detection assay (such as NanoString®) using one or more primers according to embodiments 123 to 130.

133. A method of detecting the presence of one or more bacteria selected from the genera Fenollaria, Peptoniphilus, Porphyromonas, Anaerococcus, Varibaculum, Fusobacterium, Prevotella, Propionimicrobium, Campylobacter in a biological sample from the patient.

134. A method of detecting the presence of one or more bacteria selected from the genera Fenollaria, Peptoniphilus, Porphyromonas, Anaerococcus and Prevotella in a biological sample from the patient.

135. The method according to embodiment 133 or embodiment 134, wherein the one or more bacteria are selected from: Fenollaria sporofastidiosus, Peptoniphilus rachelemmaiella, Peptoniphilus coxii, Peptoniphilus duerdenii, Peptoniphilus harei, Porphyromonas sp., Porphyromonas bobii, Porphyromonas asaccharolytica, Porphyromonas bennonis, Anaerococcus prevotii, Anaerococcus lactolyticus, Fusobacterium nucleatum, Varibaculum prostatecancerukia, Prevotella sp., Prevotella timonesis, Propionimicrobium lymphophilum and Campylobacter ureolyticus.

136. The method according to any one of embodiments 133 to 135, wherein the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis, Prevotella timonesis and Fusobacterium nucleatum.

137. The method according to any one of embodiments 133 to 135, wherein the one or more bacteria are selected from: Fenollaria sporofastidiosus, Peptoniphilus rachelemmaiella, Peptoniphilus harei, Anaerococcus prevotii, Porphyromonas bobii, Porphyromonas asaccharolytica, Prevotella timonesis and Fusobacterium nucleatum.

138. The method according to any one of embodiments 133 to 135, wherein the one or more bacteria are selected from: Fenollaria sporofastidiosus, Peptoniphilus rachelemmaiella, Anaerococcus prevotii, Porphyromonas bobii, Porphyromonas asaccharolytica, Prevotella timonesis and Fusobacterium nucleatum.

139. The method according to any one of embodiments 133 to 135, wherein the one or more bacteria are selected from: Fenollaria sporofastidiosus, Peptoniphilus harei, Anaerococcus prevotii, Porphyromonas bobii, Porphyromonas asaccharolytica, Prevotella timonesis and Fusobacterium nucleatum.

140. The method according to any one of embodiments 133 to 135, wherein the one or more bacteria are selected from: Fenollaria sporofastidiosus, Peptoniphilus rachelemmaiella, Peptoniphilus harei, Anaerococcus prevotii, Porphyromonas asaccharolytica, Prevotella timonesis and Fusobacterium nucleatum.

141. The method according to any one of embodiments 133 to 135, wherein the one or more bacteria are selected from: Fenollaria sporofastidiosus, Peptoniphilus rachelemmaiella, Peptoniphilus harei, Anaerococcus prevotii, Porphyromonas bobii, Prevotella timonesis and Fusobacterium nucleatum.

140. The method according to any one of embodiments 133 to 135, wherein the one or more bacteria are selected from: Fenollaria sporofastidiosus, Peptoniphilus rachelemmaiella, Anaerococcus prevotii, Porphyromonas bobii, Prevotella timonesis and Fusobacterium nucleatum.

141. The method according to any one of embodiments 133 to 135, wherein the one or more bacteria are selected from: Fenollaria sporofastidiosus, Peptoniphilus rachelemmaiella, Anaerococcus prevotii, Porphyromonas asaccharolytica, Prevotella timonesis and Fusobacterium nucleatum.

142. The method according to any one of embodiments 133 to 135, wherein the one or more bacteria are selected from: Fenollaria sporofastidiosus, Peptoniphilus harei, Anaerococcus prevotii, Porphyromonas bobii, Prevotella timonesis and Fusobacterium nucleatum.

143. The method according to any one of embodiments 133 to 135, wherein the one or more bacteria are selected from: Fenollaria sporofastidiosus, Peptoniphilus harei, Anaerococcus prevotii, Porphyromonas asaccharolytica, Prevotella timonesis and Fusobacterium nucleatum.

144. The method according to any one of embodiments 133 to 143, wherein the method comprises detecting the presence of at least 1, 2, 3, 4, 5, 6, 7, 8, 9 or 10 different bacterial species and/or genera.

145. The method according to any one of embodiments 133 to 144, wherein the method comprises detecting all of the species and/or genera recited in a given list.

146. The method according to any one of embodiments 133 to 135 wherein the method comprises detecting each of Fenollaria sporofastidiosus, Peptoniphilus rachelemmaiella, Peptoniphilus coxii, Peptoniphilus duerdenii, Peptoniphilus harei, Porphyromonas sp., Porphyromonas bobii, Porphyromonas asaccharolytica, Porphyromonas bennonis, Anaerococcus prevotii, Anaerococcus lactolyticus, Fusobacterium nucleatum, Varibaculum prostatecancerukia, Prevotella sp., Prevotella timonesis, Propionimicrobium lymphophilum and Campylobacter ureolyticus.

147. The method according to any one of embodiments 133 to 136, wherein the method comprises detecting each of Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis, Prevotella timonesis and Fusobacterium nucleatum.

148. The method according to any one of embodiments 133 to 147, wherein the presence of the one or more bacteria is determined using quantitative polymerase chain reaction (qPCR) or a probe-based detection assay (such as NanoString®) using one or more primers comprising a nucleotide sequence selected from the list of SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98.

149. The method according to any one of embodiments 133 to 148, wherein the presence of the one or more bacteria is determined using quantitative polymerase chain reaction (qPCR) or a probe-based detection assay (such as NanoString®) using one or more primers consisting of a nucleotide sequence selected from the list of SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98.

150. A method of providing a cancer prognosis in a patient based on the presence of one or more bacteria in a biological sample from the patient, comprising:
(a) detecting the presence of one or more bacteria selected from the genera Fenollaria, Peptoniphilus, Porphyromonas, Anaerococcus, Varibaculum, Fusobacterium, Prevotella, Propionimicrobium, Campylobacter in a biological sample from the patient, and
(b) providing a prognosis for the patient, wherein the patient has a poor prognosis if one or more of the bacteria are present in the biological sample.

151. The method according to embodiment 150, wherein the one or more bacteria are selected from: Fenollaria sporofastidiosus, Peptoniphilus rachelemmaiella, Peptoniphilus coxii, Peptoniphilus duerdenii, Peptoniphilus harei, Porphyromonas sp., Porphyromonas bobii, Porphyromonas asaccharolytica, Porphyromonas bennonis, Anaerococcus prevotii, Anaerococcus lactolyticus, Fusobacterium nucleatum, Varibaculum prostatecancerukia, Prevotella sp., Prevotella timonesis, Propionimicrobium lymphophilum and Campylobacter ureolyticus.

152. The method according to any one of embodiments 150 to 151, wherein the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis, Prevotella timonesis and Fusobacterium nucleatum.

153. The method according to any one of embodiments 150 to 151, wherein the one or more bacteria are selected from: Fenollaria sporofastidiosus, Peptoniphilus rachelemmaiella, Peptoniphilus harei, Anaerococcus prevotii, Porphyromonas bobii, Porphyromonas asaccharolytica, Prevotella timonesis and Fusobacterium nucleatum.

154. The method according to any one of embodiments 150 to 151, wherein the one or more bacteria are selected from: Fenollaria sporofastidiosus, Peptoniphilus rachelemmaiella, Anaerococcus prevotii, Porphyromonas bobii, Porphyromonas asaccharolytica, Prevotella timonesis and Fusobacterium nucleatum.

155. The method according to any one of embodiments 150 to 151, wherein the one or more bacteria are selected from: Fenollaria sporofastidiosus, Peptoniphilus harei, Anaerococcus prevotii, Porphyromonas bobii, Porphyromonas asaccharolytica, Prevotella timonesis and Fusobacterium nucleatum.

156. The method according to any one of embodiments 150 to 151, wherein the one or more bacteria are selected from: Fenollaria sporofastidiosus, Peptoniphilus rachelemmaiella, Peptoniphilus harei, Anaerococcus prevotii, Porphyromonas asaccharolytica, Prevotella timonesis and Fusobacterium nucleatum.

157. The method according to any one of embodiments 150 to 151, wherein the one or more bacteria are selected from: Fenollaria sporofastidiosus, Peptoniphilus rachelemmaiella, Peptoniphilus harei, Anaerococcus prevotii, Porphyromonas bobii, Prevotella timonesis and Fusobacterium nucleatum.

158. The method according to any one of embodiments 150 to 151, wherein the one or more bacteria are selected from: Fenollaria sporofastidiosus, Peptoniphilus rachelemmaiella, Anaerococcus prevotii, Porphyromonas bobii, Prevotella timonesis and Fusobacterium nucleatum.

159. The method according to any one of embodiments 150 to 151, wherein the one or more bacteria are selected from: Fenollaria sporofastidiosus, Peptoniphilus rachelemmaiella, Anaerococcus prevotii, Porphyromonas asaccharolytica, Prevotella timonesis and Fusobacterium nucleatum.

160. The method according to any one of embodiments 150 to 151, wherein the one or more bacteria are selected from: Fenollaria sporofastidiosus, Peptoniphilus harei, Anaerococcus prevotii, Porphyromonas bobii, Prevotella timonesis and Fusobacterium nucleatum.

161. The method according to any one of embodiments 150 to 151, wherein the one or more bacteria are selected from: Fenollaria sporofastidiosus, Peptoniphilus harei, Anaerococcus prevotii, Porphyromonas asaccharolytica, Prevotella timonesis and Fusobacterium nucleatum.

162. The method according to any one of embodiments 150 to 161, wherein the method comprises detecting the presence of at least 1, 2, 3, 4, 5, 6, 7, 8, 9 or 10 different bacterial species and/or genera.

163. The method according to any one of embodiments 150 to 162, wherein the method comprises detecting all of the species and/or genera recited in a given list.

164. A method of treating a cancer in a patient in need thereof, comprising providing a prognosis of a patient's cancer using a method as defined in any one of embodiments 150 to 163, and administering to the patient a therapy for treating cancer.

165. A method of treating cancer in a patient in need thereof, wherein the prognosis of the patient has been determined according to a method as defined in any one of embodiments 150 to 163, comprising administering to the patient a therapy for treating cancer.

166. The method according to any one of embodiments 150 to 165 wherein the cancer is selected from the list: Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Central Nervous System (Brain Cancer), Basal Cell Carcinoma, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors (Lung Cancer), Burkitt Lymphoma—see Non-Hodgkin Lymphoma, Carcinoid Tumor (Gastrointestinal), Cardiac (Heart) Tumors, Medulloblastoma and Other CNS Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Embryonal Tumors, Medulloblastoma, Endometrial Cancer, Ependymoma, Esophageal Cancer, Esthesioneuroblastoma, Ewing Sarcoma (Bone Cancer), Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Heart Tumors, Hepatocellular (Liver) Cancer, Histiocytosis, Langerhans Cell Hodgkin Lymphoma, Hypopharyngeal Cancer, Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Melanoma, Intraocular (Eye), Merkel Cell Carcinoma, Mesothelioma, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, Acute (AML), Myeloproliferative Neoplasms, Chronic Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma, Childhood Rhabdomyosarcoma (Soft Tissue Sarcoma), Childhood Vascular Tumors (Soft Tissue Sarcoma), Ewing Sarcoma (Bone Cancer), Osteosarcoma (Bone Cancer), Soft Tissue Sarcoma, Uterine Sarcoma, Sezary Syndrome, Skin Cancer, Small Cell Lung Cancer, Small Intestine Cancer, Squamous Cell Carcinoma, Squamous Neck Cancer with Occult Primary, Metastatic, Stomach (Gastric) Cancer, T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Ureter and Renal Pelvis, Transitional Cell Cancer (Kidney (Renal Cell) Cancer, Urethral Cancer, Uterine Cancer, Vaginal Cancer, Vascular Tumors, Vulvar Cancer, Wilms Tumor, Myxofibrosarcoma, Leiomyosarcoma, Pleomorphic Sarcoma and Hepatopancreatobiliary cancer.

167. The method according to any one of embodiments 150 to 166, wherein the cancer is prostate cancer.

168. A nucleotide primer comprising a sequence selected from the list of SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98.

169. The nucleotide primer according to embodiment 168, consisting of a sequence selected from the list of SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98.

170. A kit for determining the presence of one or more bacteria in a biological sample, comprising means for detecting one or more bacteria selected from the genera Fenollaria, Peptoniphilus, Porphyromonas, Anaerococcus, Varibaculum, Fusobacterium, Prevotella, Propionimicrobium, Campylobacter in a biological sample.

171. The kit according to embodiment 170, wherein the one or more bacteria are selected from: Fenollaria sporofastidiosus, Peptoniphilus rachelemmaiella, Peptoniphilus coxii, Peptoniphilus duerdenii, Peptoniphilus harei, Porphyromonas sp., Porphyromonas bobii, Porphyromonas asaccharolytica, Porphyromonas bennonis, Anaerococcus prevotii, Anaerococcus lactolyticus, Fusobacterium nucleatum, Varibaculum prostatecancerukia, Prevotella sp., Prevotella timonesis, Propionimicrobium lymphophilum and Campylobacter ureolyticus.

172. A computer apparatus configured to perform a method according to any one of embodiments 133 to 171 or, a computer readable medium programmed to perform a method according to any one of embodiments 133 to 171.

173. A method of detecting the presence of Prevotella timonesis in a biological sample.

174. The method according to embodiment 174, wherein the presence of the bacteria is determined using quantitative polymerase chain reaction (qPCR) or a probe-based detection assay (such as NanoString®) using one or more primers comprising a nucleotide sequence selected from SEQ ID NO: 95, SEQ ID NO: 96, SEQ ID NO: 97 and SEQ ID NO: 98.

175. The method according to embodiment 174, wherein the presence of the bacteria is determined using quantitative polymerase chain reaction (qPCR) or a probe-based detection assay (such as NanoString®) using one or more primers comprising a nucleotide sequence selected from SEQ ID NO: 95, SEQ ID NO: 96, SEQ ID NO: 97 and SEQ ID NO: 98.

176. A method of providing a cancer prognosis in a patient based on the presence of Prevotella timonesis in a biological sample from the patient, comprising:
(a) detecting the presence of Prevotella timonesis in a biological sample from the patient, and
(b) providing a prognosis for the patient, wherein the patient has a poor prognosis if Prevotella timonesis is present in the biological sample.

177. A method of treating a cancer in a patient in need thereof, comprising providing a prognosis of a patient's cancer using a method as defined in embodiment 176, and administering to the patient a therapy for treating cancer.

178. A method of treating cancer in a patient in need thereof, wherein the prognosis of the patient has been determined according to a method as defined in embodiment 177, comprising administering to the patient a therapy for treating cancer.

179. The method according to any one of embodiments 176 to 178, wherein the cancer is prostate cancer.

180. A nucleotide primer comprising a sequence between 5 and 25 nucleotides in length that is substantially complementary or reverse complementary to a corresponding length portion of Prevotella timonesis.

181. A nucleotide primer comprising a sequence between 5 and 25 nucleotides in length that is substantially complementary or reverse complementary to a corresponding length portion of Prevotella timonesis 4401737.

182. A nucleotide primer comprising a sequence between 5 and 25 nucleotides in length that is substantially complementary or reverse complementary to a corresponding length portion of a sequence selected from the list consisting of SEQ ID NOs:99-120.

183. The nucleotide primer according to embodiment 182, wherein the primer is capable of detecting the presence of Prevotella timonesis in a biological sample.

184. The method according to any one of embodiments 133 to 167 or 176 to 179, wherein the presence of the one or more bacteria is determined using quantitative polymerase chain reaction (qPCR) or a probe-based detection assay (such as NanoString®) using one or more primers according to embodiments 180 to 183.

185. A kit for determining the presence of Prevotella timonesis in a biological sample.

186. The kit according to embodiment 185, wherein the means for detecting is quantitative polymerase chain reaction (qPCR) or a probe-based detection assay (such as NanoString®).

187. The kit according to embodiment 186, wherein the means for detecting is quantitative polymerase chain reaction (qPCR) or a probe-based detection assay (such as NanoString®) and the kit comprises one or more primers comprising a nucleotide sequence selected from SEQ ID NO: 95, SEQ ID NO: 96, SEQ ID NO: 97 and SEQ ID NO: 98.

188. The kit according to embodiment 186 and 187, wherein the means for detecting is quantitative polymerase chain reaction (qPCR) or a probe-based detection assay (such as NanoString®) and the kit comprises one or more primers consisting of a nucleotide sequence selected from SEQ ID NO: 95, SEQ ID NO: 96, SEQ ID NO: 97 and SEQ ID NO: 98.

189. The kit according to embodiment 187 and 187, wherein the means for detecting is quantitative polymerase chain reaction (qPCR) or a probe-based detection assay (such as NanoString®) and the kit comprises one or more primers comprising a sequence between 5 and 25 nucleotides in length that is substantially complementary or reverse complementary to a corresponding length portion of a sequence selected from the list consisting of SEQ ID NOs:99-120.

It will be apparent to those skilled in the art that various modifications and variations can be made to the compositions and processes of this invention. Thus, it is intended that the present invention cover such modifications and variations, provided they come within the scope of the appended claims and their equivalents.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

[1] Cancer Research UK. Prostate cancer incidence statistics [Internet]. 2019 [cited 2019 Jun. 29]. Available from: http://www.cancerresearchuk.org/health-professional/cancer-statistics/statistics-by-cancer-type/prostate-cancer/incidence
[2] Sanda M G, Cadeddu J A, Kirkby E, Chen R C, Crispino T, Fontanarosa J, et al. Clinically Localized Prostate Cancer: AUA/ASTRO/SUO Guideline. Part I: Risk Stratification, Shared Decision Making, and Care Options. Journal of Urology [Internet]. 2018; 199(3):683-90. Available from: https://doi.org/10.1016/j.juro.2017.11.095
[3] Cornford P, Bellmunt J, Bolla M, Briers E, De Santis M, Gross T, et al. EAU-ESTRO-SIOG Guidelines on Prostate Cancer. Part II: Treatment of Relapsing, Metastatic, and Castration-Resistant Prostate Cancer. European Urology [Internet]. 2017; 71(4):630-42. Available from: http://dx.doi.org/10.1016/j.euroro.2016.08.002
[4] National Institute for Health and Care Excellence. Prostate cancer: diagnosis and management (update). NICE.
[5]D'Amico A V., Whittington R, Bruce Malkowicz S, et al. Biochemical outcome after radical prostatectomy, external beam radiation therapy, or interstitial radiation therapy for clinically localized prostate cancer. J Am Med Assoc. 1998; 280(11):969-974. doi:10.1001/jama.280.11.969.
[6] Epstein J I, Zelefsky M J, Sjoberg D D, et al. A Contemporary Prostate Cancer Grading System: A Validated Alternative to the Gleason Score. Eur Urol. 2016; 69(3):428-435. doi:10.1016/j.eururo.2015.06.046.
[7] Sanda M G, Cadeddu J A, Kirkby E, et al. Clinically Localized Prostate Cancer: AUA/ASTRO/SUO Guideline. Part I: Risk Stratification, Shared Decision Making, and Care Options. J Urol. 2018; 199(3):683-690. doi:10.1016/j.juro.2017.11.095.
[8] Mottet N, Bellmunt J, Bolla M, et al. EAU-ESTRO-SIOG Guidelines on Prostate Cancer. Part 1: Screening, Diagnosis, and Local Treatment with Curative Intent. Eur Urol. 2017; 71(4):618-629. doi:10.1016/j.eururo.2016.08.003.
[9] National Institute for Health and Care Excellence. Prostate Cancer: Diagnosis and Treatment.; 2014.
[10] Selvadurai E D, Singhera M, Thomas K, et al. Medium-term outcomes of active surveillance for localised prostate cancer. Eur Urol. 2013; 64(6):981-987. doi:10.1016/j.eururo.2013.02.020.
[11] Cooperberg M R, Freedland S J, Pasta D J, et al. Multiinstitutional validation of the UCSF cancer of the prostate risk assessment for prediction of recurrence after radical prostatectomy. Cancer. 2006; 107(10):2384-2391. doi:10.1002/cncr.22262.
[12] Brajtbord J S, Leapman M S, Cooperberg M R. The CAPRA Score at 10 Years: Contemporary Perspectives and Analysis of Supporting Studies. Eur Urol. 2017; 71(5):705-709. doi:10.1016/j.eururo.2016.08.065.
[13] Martin R M, Donovan J L, Turner E L, Metcalfe C, Young G J, Walsh E I, et al. Effect of a low-intensity PSA-based screening intervention on prostate cancer mortality: The CAP randomized clinical trial. JAMA—Journal of the American Medical Association [Internet]. 2018 March; 319(9):883-95. Available from:
[14] Donovan J L, Hamdy F C, Lane J A, Mason M, Metcalfe C, Walsh E, et al. Patient-Reported Outcomes after Monitoring, Surgery, or Radiotherapy for Prostate Cancer. New England Journal of Medicine [Internet]. 2016 October; 375(15):1425-37. Available from: http:www.nejrn.org/doi/10.1056/NEJMoa1606221
[15] Ahmed H U, EI-Shater Bosaily A, Brown L C, Gabe R, Kaplan R, Parmar M K, et al. Diagnostic accuracy of multi-parametric MRI and TRUS biopsy in prostate cancer (PROMIS): a paired validating confirmatory study. The Lancet [Internet]. 2017 February; 389(10071):815-22. Available from: http:www.ncbi.nirn.nih.gov/pubmed/26110982
[16] Pepe P, Pennisi M. Gleason score stratification according to age at diagnosis in 1028 men. Wspolczesna Onkologia [Internet]. 2015; 19(6):471-3. Available from: https://www.ncbi.nlm.nih.gov/pmc/articles/PMC4731454/pdf/WO-19-26452.pdf
[17] Sonn G A, Fan R E, Ghanouni P, Wang N N, Brooks J D, Loening A M, et al. Prostate Magnetic Resonance Imaging Interpretation Varies Substantially Across Radiologists [Internet]. Elsevier; 2018. Available from: https://www.sciencedirect.com/science/article/pii/S2405456917302663
[18] Walz J. The “PROMIS” of Magnetic Resonance Imaging Cost Effectiveness in Prostate Cancer Diagnosis?European Urology [Internet]. 2018 January; 73(1):31-2. Available from: http://www.ncbi.nlm.nih.gov/pubmed/28965689
[19] Hessels D, Klein Gunnewiek J M, Van Oort I, Karthaus H F, Van Leenders G J, Van Balken B, et al. DD3PCA3-based molecular urine analysis for the diagnosis of prostate cancer. European Urology [Internet]. 2003 July; 44(1):8-16. Available from: http://llnkinghun.eisevier.com/retneve/pii/S030228380300201X
[20] Van Neste L, Hendriks R J, Dijkstra S, Trooskens G, Cornel E B, Jannink S A, et al. Detection of High-grade Prostate Cancer Using a Urinary Molecular Biomarker-Based Risk Score. European Urology. 2016; 70(5):740-8.
[21] McKiernan J, Donovan M J, O'Neill V, Bentink S, Noerholm M, Belzer S, et al. A novel urine exosome gene expression assay to predict high-grade prostate cancer at initial biopsy. JAMA Oncology [Internet]. 2016 July; 2(7):882-9. Available from: http://oncology.jamanetwork.com/article.aspx?doi=10.1001/jamaoncol2016.0097
[22] Jones, A. L. & Chinegwundoh, F. Update on prostate cancer in black men within the UK. Ecancermedicalscience 8, 455 (2014).
[23] Kimura, T. & Egawa, S. Epidemiology of prostate cancer in Asian countries. Int J Urol 25, 524-531 (2018).
[24] Hjelmborg, J. B., et al. The heritability of prostate cancer in the Nordic Twin Study of Cancer. Cancer Epidemiol Biomarkers Prev 23, 2303-2310 (2014).
[25] Wheeler, K. M. & Liss, M. A. The Microbiome and Prostate Cancer Risk. Curr Urol Rep 20, 66 (2019).
[26] Shrestha, E., et al. Profiling the Urinary Microbiome in Men with Positive versus Negative Biopsies for Prostate Cancer. J Urol 199, 161-171 (2018).
[27] Puhr, M., et al. Inflammation, Microbiota, and Prostate Cancer. Eur Urol Focus 2, 374-382 (2016).
[28] Liss, M. A., et al. Metabolic Biosynthesis Pathways Identified from Fecal Microbiome Associated with Prostate Cancer. Eur Urol 74, 575-582 (2018).
[29] National Institute for Health and Care Excellence. Prostate Cancer: diagnosis and management. National Institute for Health and Care Excellence, London, (2019).
[30] Blacklock, N.J. Anatomical factors in prostatitis. Br J Urol 46, 47-54 (1974).
[31] Kirby, R. S., Lowe, D., Bultitude, M. I. & Shuttleworth, K. E. Intra-prostatic urinary reflux: an aetiological factor in a bacterial prostatitis. Br J Urol 54, 729-731 (1982).
[32] Butel, R. & Ball, R. The distribution of BCG prostatitis: A clue for pathogenetic processes?Prostate 78, 1134-1139 (2018).
[33]O'Reilly, E., et al. epiCaPture: A Urine DNA Methylation Test for Early Detection of Aggressive Prostate Cancer. JCO Precis Oncol 3, 1-18 (2019).
[34] Connell, S. P., et al. A Four-Group Urine Risk Classifier for Predicting Outcome in Prostate Cancer Patients. BJU Int 124, 609-620 (2019).
[35] Connell, S. P., et al. Development of a multivariable risk model integrating urinary cell DNA methylation and cell-free RNA data for the detection of significant prostate cancer. Prostate 80, 547-558 (2020).
[36] Sha, S., Ni, L., Stefil, M., Dixon, M. & Mouraviev, V. The human gastrointestinal microbiota and prostate cancer development and treatment. Investig Clin Urol 61, S43-S50 (2020).
[37] Alanee, S., et al. A prospective study to examine the association of the urinary and fecal microbiota with prostate cancer diagnosis after transrectal biopsy of the prostate using 16sRNA gene analysis. Prostate 79, 81-87 (2019).
[38] Banerjee, S., et al. Microbiome signatures in prostate cancer. Carcinogenesis 40, 749-764 (2019).
[39] Garrett, W. S. Cancer and the microbiota. Science 348, 80-86 (2015).
[40] Chang, A. H. & Parsonnet, J. Role of bacteria in oncogenesis. Clin Microbiol Rev 23, 837-857 (2010).
[41] Hug, L. A., et al. A new view of the tree of life. Nat Microbiol 1, 16048 (2016).
[42] Sfanos, K. S. et al., The inflammatory microenvironment and microbiome in prostate cancer development, Nature Reviews Urology volume 15, pages 11-24 (2018)
[43] Nejman, D., et al. The human tumor microbiome is composed of tumor type-specific intracellular bacteria. Science 368, 973-980 (2020).
[44] Poore, G. D., et al. Microbiome analyses of blood and tissues suggest cancer diagnostic approach. Nature 579, 567-574 (2020).
[45] Zapatka, M., et al. The landscape of viral associations in human cancers. Nat Genet 52, 320-330 (2020).
[46] Yu, Z. & Morrison, M. Improved extraction of PCR-quality community DNA from digesta and fecal samples. Biotechniques 36, 808-812 (2004).
[47] Yarza, P., et al. Uniting the classification of cultured and uncultured bacteria and archaea using 16S rRNA gene sequences. Nat Rev Microbiol 12, 635-645 (2014).
[48] Gaspar, J. M. & Thomas, W. K. FlowClus: efficiently filtering and denoising pyrosequenced amplicons. BMC Bioinformatics 16, 105 (2015).
[49] Edgar, R. C. Search and clustering orders of magnitude faster than BLAST. Bioinformatics 26, 2460-2461 (2010).
[50] Cole, J. R., et al. Ribosomal Database Project: data and tools for high throughput rRNA analysis. Nucleic Acids Res 42, D633-642 (2014).
[51] Quast, C., et al. The SILVA ribosomal RNA gene database project: improved data processing and web-based tools. Nucleic Acids Res 41, D590-596 (2013).
[52] McMurdie, P. J. & Holmes, S. phyloseq: an R package for reproducible interactive analysis and graphics of microbiome census data. PLoS One 8, e61217 (2013).
[53] Ashton, P. M., et al. MinION nanopore sequencing identifies the position and structure of a bacterial antibiotic resistance island. Nat Biotechnol 33, 296-300 (2015).
[54] Bolger, A. M., Lohse, M. & Usadel, B. Trimmomatic: a flexible trimmer for Illumina sequence data. Bioinformatics 30, 2114-2120 (2014).
[55] Bankevich, A., et al. SPAdes: a new genome assembly algorithm and its applications to single-cell sequencing. J Comput Biol 19, 455-477 (2012).
[56] Simao, F. A., Waterhouse, R. M., loannidis, P., Kriventseva, E. V. & Zdobnov, E. M. BUSCO: assessing genome assembly and annotation completeness with single-copy orthologs. Bioinformatics 31, 3210-3212 (2015).
[57] Seemann, T. Prokka: rapid prokaryotic genome annotation. Bioinformatics 30, 2068-2069 (2014).
[58] Quevillon, E., et al. InterProScan: protein domains identifier. Nucleic Acids Res 33, W116-120 (2005).
[59] Katoh, K. & Standley, D. M. MAFFT multiple sequence alignment software version 7: improvements in performance and usability. Mol Biol Evol 30, 772-780 (2013).
[60] Capella-Gutierrez, S., Silla-Martinez, J. M. & Gabaldon, T. trimAl: a tool for automated alignment trimming in large-scale phylogenetic analyses. Bioinformatics 25, 1972-1973 (2009).
[61] Stamatakis, A. RAxML version 8: a tool for phylogenetic analysis and post-analysis of large phylogenies. Bioinformatics 30, 1312-1313 (2014).
[62] Lee, I., Ouk Kim, Y., Park, S. C. & Chun, J. OrthoANI: An improved algorithm and software for calculating average nucleotide identity. Int J Syst Evol Microbiol 66, 1100-1103 (2016).
[63] Gihawi, A., et al. SEPATH: benchmarking the search for pathogens in human tissue whole genome sequence data leads to template pipelines. Genome Biol 20, 208 (2019).
[64] Parker, C., Tindall, B, Garrity G M. International Code of Nomenclature of Prokaryotes, Prokaryotic Code (2008 Revision). Int J Syst Evol Microbiol 69, S1-S111 (2019).
[65] White C E, Winans S C. Cell-cell communication in the plant pathogen Agrobacterium tumefaciens. Philos Trans R Soc Lond B Biol Sci. 2007; 362:1135-48.
[66] Aragon I M, Herrera-Imbroda B, Queipo-Ortuno M I, Castillo E, Del Moral J S, Gomez-Millan J, et al. The Urinary Tract Microbiome in Health and Disease. Eur Urol Focus. 2018; 4:128-38.
[67] Eisenhofer R, Minich J J, Marotz C, Cooper A, Knight R, Weyrich L S. Contamination in Low Microbial Biomass Microbiome Studies: Issues and Recommendations. Trends Microbiol. 2019; 27:105-17.
[68] Han Y W. Fusobacterium nucleatum: a commensal-turned pathogen. Curr Opin Microbiol. 2015; 23:141-7.
[69] Song Y, Finegold, S. M. Peptostreptococcus, Finegoldia, Anaerococcus, Peptoniphilus, Veillonella, and Other Anaerobic Cocci. In: James H. Jorgensen K C C, Guido Funke, Michael A. Pfaller, Marie Louise Landry, Sandra S. Richter, David W. Warnock, editor. Manual of Clinical Microbiology, 11th Edition 2015. p. 909-19.
[70] Salter, S. J., et al. Reagent and laboratory contamination can critically impact sequence-based microbiome analyses. BMC Biol 12, 87 (2014).
[71] Caporaso, J. G., et al. QIIME allows analysis of high-throughput community sequencing data. Nature Methods 7, 335 (2010).
[72] Browne, H. P., et al. Culturing of ‘unculturable’ human microbiota reveals novel taxa and extensive sporulation. Nature 533, 543-546 (2016).
[73] Schloss, P. D. & Handelsman, J. Introducing DOTUR, a computer program for defining operational taxonomic units and estimating species richness. Appl Environ Microbiol 71, 1501-1506 (2005).
[74] Audirac-Chalifour, A., et al. Cervical Microbiome and Cytokine Profile at Various Stages of Cervical Cancer: A Pilot Study. PLoS One 11, e0153274 (2016).
[75] Flanagan, L., et al. Fusobacterium nucleatum associates with stages of colorectal neoplasia development, colorectal cancer and disease outcome. Eur J Clin Microbiol Infect Dis 33, 1381-1390 (2014).
[76] Sfanos, K. S., et al. A molecular analysis of prokaryotic and viral DNA sequences in prostate tissue from patients with prostate cancer indicates the presence of multiple and diverse microorganisms. Prostate 68, 306-320 (2008).
[77] Attard, G., et al. Phase I clinical trial of a selective inhibitor of CYP17, abiraterone acetate, confirms that castration-resistant prostate cancer commonly remains hormone driven. J Clin Oncol 26, 4563-4571 (2008).
[78] Burdelski, C., et al. Overexpression of thymidylate synthase (TYMS) is associated with aggressive tumor features and early PSA recurrence in prostate cancer. Oncotarget 6, 8377-8387 (2015).
[79] Peters, G. J., et al. Induction of thymidylate synthase as a 5-fluorouracil resistance mechanism. Biochim Biophys Acta 1587, 194-205 (2002).
[80] Spicer, J., et al. Phase I I study of oral capecitabine in patients with hormone-refractory prostate cancer. Prostate Cancer Prostatic Dis 8, 364-368 (2005).
[81] Kdadra, M., Hockner, S., Leung, H., Kremer, W. & Schiffer, E. Metabolomics Biomarkers of Prostate Cancer: A Systematic Review. Diagnostics (Basel) 9(2019).
[82] Locasale, J. W. Serine, glycine and one-carbon units: cancer metabolism in full circle. Nat Rev Cancer 13, 572-583 (2013).
[83] Lin, S., Hanson, R. E. & Cronan, J. E. Biotin synthesis begins by hijacking the fatty acid synthetic pathway. Nat Chem Biol 6, 682-688 (2010).

1. A method of providing a cancer prognosis in a patient based on the presence of one or more bacteria in a biological sample from the patient, comprising:
(a) detecting the presence of one or more bacteria selected from the genera Fenollaria, Ezakiella, Peptoniphilus, Porphyromonas, Anaerococcus and Fusobacterium in a biological sample from the patient,
(b) providing a prognosis for the patient, wherein the patient has a poor prognosis if one or more of the bacteria are present in the biological sample.

2. The method according to claim 1 wherein the one or more bacteria is selected from the genera Fenollaria, Peptoniphilus, Porphyromonas, Anaerococcus and Fusobacterium in a biological sample from the patient,

3. The method according to claim 1 or 2 comprising detecting the presence of bacteria from each of the genera Fenollaria, Peptoniphilus, Porphyromonas, Anaerococcus and Fusobacterium in a biological sample from the patient,

4. The method according to any one of claims 1 to 3, wherein the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

5. The method according to any one of claims 1 to 4, wherein one or more of the bacteria are selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

6. The method according to any one of claims 1 to 5, wherein one or more of the bacteria are selected from: Fenollaria sporofastidiosus (identified by ECACC accession number 21092201 or DSMZ accession number DSM 34056), Peptoniphilus rachelemmaiella (identified by ECACC accession number 21092202 or DSMZ accession number DSM 34055) and Porphyromonas bobii (identified by ECACC accession number 21092204 or DSMZ accession number DSM 34063).

7. The method according to any one of claims 1 to 6, wherein a poor prognosis is associated with an increased risk of elevated rates of metastases.

8. The method according to any one of claims 1 to 7, wherein the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample).

9. The method according to any one of claims 1 to 8 wherein the biological sample is a urine sample.

10. The method according to any one of claims 1 to 9, wherein the method is used to determine whether a patient should be biopsied.

11. The method according to any one of claims 1 to 10, wherein the biological sample is processed prior to determining the presence of the one or more bacteria in the biological sample.

12. The method according to any one of claims 1 to 11, wherein the presence of the one or more bacteria is determined using quantitative polymerase chain reaction (qPCR) or a probe-based detection assay (such as NanoString®).

13. The method according to claim 12, wherein the presence of the one or more bacteria is determined using quantitative polymerase chain reaction (qPCR) or a probe-based detection assay (such as NanoString®) using one or more primers comprising a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

14. A method of detecting the presence of one or more bacteria selected from the genera Fenollaria, Peptoniphilus, Porphyromonas, Anaerococcus and Fusobacterium in a biological sample from the patient.

15. The method according to claim 14, wherein the one or more bacteria are selected from: Fenollaria sporofastidiosus, Peptoniphilus rachelemmaiella, Peptoniphilus harei, Peptoniphilus coxii, Anaerococcus prevotii, Anaerococcus lactolyticus, Porphyromonas bobii, Porphyromonas asaccharolytica, Porphyromonas bennonis and Fusobacterium nucleatum.

16. The method according to claim 14 or claim 15, wherein one or more of the bacteria are selected from: Fenollaria sporofastidiosus, Peptoniphilus rachelemmaiella, Peptoniphilus harei, Anaerococcus prevotii, Porphyromonas bobii, Porphyromonas asaccharolytica and Fusobacterium nucleatum.

17. The method according to any one of claims 14 to 16, wherein one or more of the bacteria are selected from: Fenollaria sporofastidiosus (identified by ECACC accession number 21092201 or DSMZ accession number DSM 34056), Peptoniphilus rachelemmaiella (identified by ECACC accession number 21092202 or DSMZ accession number DSM 34055) and Porphyromonas bobii (identified by ECACC accession number 21092204 or DSMZ accession number DSM 34063).

18. The method according to any one of claims 14 to 17, wherein the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample).

19. The method according to any one of claims 14 to 18 wherein the biological sample is a urine sample.

20. The method according to any one of claims 14 to 19, wherein the presence of the one or more bacteria is determined using quantitative polymerase chain reaction (qPCR) or a probe-based detection assay (such as NanoString®).

21. The method according to any one of claims 14 to 20, wherein the presence of the one or more bacteria is determined using quantitative polymerase chain reaction (qPCR) or a probe-based detection assay (such as NanoString®) using one or more primers comprising a nucleotide sequence selected from the list SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

22. The method according to any preceding claim wherein the sample is from a human.

23. A method of treating a cancer in a patient in need thereof, comprising providing a prognosis of a patient's cancer using a method as defined in any one of claims 1 to 12, and administering to the patient a therapy for treating cancer.

24. The method according to any one of claims 1 to 12 and 22Error!Reference source not found., wherein the cancer is prostate cancer.

25. The method according to any one of claims 22 to 23, wherein the therapy for cancer comprises surgery, brachytherapy, active surveillance, chemotherapy, hormone therapy, immunotherapy and/or radiotherapy.

26. A cell culture comprising bacteria Varibaculum prostatecancerukia identified by ECACC deposit number 21092203 or DSMZ accession number DSM 34057.

27. A cell culture comprising bacteria Fenollaria sporofastidiosus identified by ECACC deposit number 21092201 or DSMZ accession number DSM 34056.

28. A cell culture comprising bacteria Peptoniphilus rachelemmaiella identified by ECACC deposit number 21092202 or DSMZ accession number DSM 34055.

29. A cell culture comprising bacteria Porphyromonas bobii identified by ECACC deposit number 21092204 or DSMZ accession number DSM 34063.

30. Use of a cell culture according to any one of claims 26 to 29 in providing a prognosis of cancer in a patient.
